(19) United States
(12) Patent Application Publication
(10) Pub. No.: US 2025/0215501 A1
(43) Pub. Date: Jul. 3, 2025

(54) METHODS OF CANCER PROGNOSIS

(71) Applicant: CANCER RESEARCH TECHNOLOGY LIMITED, Greater London (GB)

(72) Inventors: Dan Woodcock, Oxford (GB); David Wedge, Manchester (GB); Colin Cooper, Norwich (GB)

(21) Appl. No.: 18/695,711

(22) PCT Filed: Sep. 27, 2022
(86) PCT No.: PCT/GB2022/052435

§ 371 Date: Mar. 26, 2024

(30) Foreign Application Priority Data
Sep. 27, 2021 (GB) 2113759.1

(51) Int. Cl. C12Q 1/6886

(52) U.S. Cl. CPC C12Q 1/6886; C12Q 2600/118; C12Q 2600/156

(57) ABSTRACT

We describe methods for stratifying a subject affected by prostate cancer into one of two prognostic groups. The first prognostic group may be termed an Alternative-evotype group and the second prognostic group may be termed a Canonical-evotype group. The Canonical-evotype group comprises tumours which evolve over the same or different trajectories to a form of the cancer which may be considered to have a standard form. The Alternative-evotype group comprises tumours which evolve over the same or different trajectories to a non-standard or alternative form of the cancer. One of the methods comprises: analysing, using DNA and/or RNA sequencing, a biological sample obtained from the subject with cancer or metastatic disease, identifying genetic aberrations in the biological sample, and classifying the subject in a first or in a second prognostic group based on the presence of the genetic aberrations.

### FIELD

The invention relates to a method of stratifying cancer patients into prognostic groups, particularly prostate cancer patients.

### BACKGROUND

Tumour evolution is a dynamic process (1) involving the accumulation of genetic alterations that lead to pathological phenotypes (2). In some cancer types, genomic and expression changes that characterise these phenotypes have been used to develop clinically-actionable classification frameworks (3-6). For localised prostate cancer, stratification based on the presence of specific molecular alterations (7), combinations of alterations (8) or gene expression profiles (9) have been proposed.

However, detailed investigations of prostate cancer genomes (10, 11, 12) have shown substantial heterogeneity in the occurrence of genomic variants that confounds the clinical utility of these simple classification schemes (13). More recent studies have distinguished events that are likely to occur early or late in prostate cancer evolution that can be informative of early-onset (14) and aggressive disease (15). However, the role of evolution in the development of disease types remains largely unexplored.

As such there is a need for methods which can classify cancer types in a clinically useful manner.

### SUMMARY

The development of cancer is an evolutionary process, but the factors that promote the emergence of different disease types remain poorly understood. The present inventors applied three statistical and machine-learning methods to genomic measurements from 159 prostate cancer patients, each of which identified a different aspect of tumour evolution. Integrating these results revealed that the tumours followed evolutionary trajectories that converge to two forms of the disease, defined as the Canonical and Alternative evolutionary types (evotypes). The Canonical-evotype tumour evolves to a standard form of the disease with normal prognosis. The Alternative-evotype tumour evolves to a different form of the disease and has poorer prognosis.

Statistical modelling revealed multiple routes to each evotype that were dependent on the stochastic acquisition of complementary genetic alterations. Classification by evotype therefore reflects the influence of several interacting factors and provides a powerful new paradigm for cancer stratification.

As such, according to the present invention there are provided methods as set forth in the appended claims. Other features of the invention will be apparent from the dependent claims, and the description which follows.

### Methods of Stratification

According to a first aspect of the invention, there is provided a method for stratifying a subject into one of two prognostic groups, the method comprising: analysing, using DNA and/or RNA sequencing, a biological sample obtained from the subject with cancer or metastatic disease, determining the location of double stranded DNA breaks relative to androgen receptor binding sites (ARBS), and classifying the cancer patient in a first prognostic group when the determined locations are less frequently proximal to androgen receptor binding sites (ARBS) than expected and classifying the cancer patient in a second prognostic group when the determined locations are more frequently proximal to androgen receptor binding sites (ARBS) than expected. The method may further comprise classifying the cancer patient in the second prognostic group when there is no statistically significant difference between the proximity of the determined locations to the ARBS and the expected proximity of locations to the ARBS.

The first prognostic group may be termed an Alternative-evotype group and the second prognostic group may be termed a Canonical-evotype group. The Canonical-evotype group comprises tumours which evolve over the same or different trajectories to a form of the cancer which may be considered to have a standard form. The Alternative-evotype group comprises tumours which evolve over the same or different trajectories to a non-standard or alternative form of the cancer. Tumours which evolve to the alternative form have a poorer prognosis than tumours which evolve to the standard form. A poor prognosis may be characterised by a reduced likelihood of progression free survival, determined through time to biochemical recurrence, defined as a prostate-specific antigen (PSA) level greater than 0.2 ng/ml of blood in the period after radical treatment. Alternatively, a poor prognosis may be characterised by the observation of metastasis or death. Radical treatment may include radical prostatectomy or radiotherapy. Tumours which evolve to the standard form have a standard prognosis which may be characterised by an increased likelihood of progression free survival. For example, a subject may have progression free survival for up to 120 months, or 100 months, or 80 months, or 60 months, or 40 months, or 24 months, or 12 months.

By using ARBS as described above, this method of classification may be termed an ARBS classification. An ARBS is a binding site for an androgen receptor protein. “Androgen receptor” (AR) is a DNA-binding transcription factor that regulates gene expression. Given that AR is widely expressed in many cells and tissues, AR has a diverse range of biological actions including important roles in the development and maintenance of the reproductive, musculoskeletal, cardiovascular, immune, neural and haemopoietic systems. AR signalling may also be involved in the development of tumours in the prostate, bladder, liver, kidney and lung. The AR has also been identified as having a key role in prostate cancer in particular castration resistant prostate cancer. AR is a member of the steroid hormone receptors, a group of steroid-inducible transcription factors sharing a consensus DNA-binding motif.

Methods to identify transcription factor binding sites, such as Androgen receptor Binding Sites (ARBS), are known to the skilled person, for example chromatin immunoprecipitation assays combined with sequencing (ChIP-seq) is a common method for identifying genome wide DNA binding sites for transcription factors. Following ChIP protocols, DNA-bound protein is immunoprecipitated using a specific antibody. The bound DNA is then coprecipitated, purified, and sequenced. The sequencing may be performed using next generation sequencing methods (NGS). In one example, ARBS may be identified using the processed ChIP-seq data targeting AR for 13 primary prostate cancer tumours from Gene Expression Omnibus (accession GSE70079, M. M. Pomerantz, et al., Nature Genetics 47, 1346 (2015)). This ChIP-seq data may be amalgamated for use as locations of the ARBS.

The proximity of the determined locations to ARBS may be compared to the proximity of a baseline (or expected) distribution of the breakpoint locations to ARBS to obtain an ARBS score which is used to classify the patient into one of the first and second prognostic groups. The baseline distribution may be a random distribution of the locations of double stranded DNA breakpoints across the genome. The baseline distribution may be identified by a permutation approach. For example, the baseline distribution may be determined from a plurality of samples, e.g. the samples identified above in Nature Genetics. The observed breakpoints in the sample data across the genome (e.g. GRCh37) may be randomly shuffled, for example masked for assembly gaps (AGAPS mask) and intra-contig ambiguities (AMB mask) 1000 times using the R package RegioneR (67) to obtain the baseline distribution.

A double stranded DNA break may be considered to be relatively proximal to an ARBS when the break is less than a threshold number of base pairs (e.g. 20,000 bps) from an ARBS and may be considered to be distal when the break is more than or equal to a threshold number of base pairs (e.g. 20,000 bps) from an ARBS. The method may comprise determining the proportion of determined (e.g. observed) locations which are less than the plurality of base pairs from an ARBS. The method may also comprise obtaining the proportion of locations in the baseline distribution which are less than the plurality of base pairs from an ARBS. The method may comprise normalising the determined proportion by the obtained proportion to obtain the ARBS score to determine whether the determined locations are more frequently proximal or less frequently proximal to androgen receptor binding sites than expected. When the proportion of determined locations which are relatively proximal is greater than an upper threshold (e.g. 97.5%) of the proportion of locations in the baseline distribution which are relatively proximal, the determined locations may be considered to be more frequently proximal than the predetermined locations (i.e. the tumour may be classified as enriched). When the ARBS score is used, the tumour may be classified as enriched when the ARBS score is above an upper threshold. When the proportion of determined locations which are relatively proximal is less than a lower threshold (e.g. 2.5%) of the proportion of locations in the baseline distribution which are relatively proximal, the determined locations may be considered to be less frequently proximal than the predetermined locations (i.e. the tumour may be classified as depleted). When the ARBS score is used, the tumour may be classified as depleted when the ARBS score is below a lower threshold. If neither of these conditions are met, there may be considered to be no statistically significant difference (i.e. the tumour may be classified as indeterminate).

Without wishing to be bound by theory, it is hypothesised that in the Alternative evotype certain genetic alterations cause altered androgen receptor binding, that promotes DNA breaks in positions which are distal from the ARBS, leading to different copy number changes and giving rise to a mechanistically different form of the cancer (for example prostate cancer) that has a poor prognosis. In contrast in the Canonical evotype genetic alterations occur which have double stranded DNA breaks proximal to androgen receptor binding sites and results in a standard progression of prostate cancer. In other words, the ARBS score (i.e. double stranded DNA breaks) may be used in the prognosis of prostate cancer.

The ARBS score may be considered to be representative of a genomic aberration in the sample. The present inventors have identified further genomic aberrations which are characteristic of the first and second prognostic groups, i.e. of the Alternative evotype and the Canonical evotype of prostate cancer. A genomic aberration as used herein may thus be defined is any alteration of a genomic sequence, for example a deletion, insertion, inversion, duplication, loss of heterozygosity, gain of heterozygosity, DNA breakage, gene fusion, any other chromosomal mutation, or a measure of such alterations, for example PGA (percentage genome altered), number of breakpoints, ARBS score. Genetic anomalies that convey complementary information that can be used to distinguish prognostic groups can be identified with a neural network (e.g. a restricted Boltzmann machine or autoencoder) and can be grouped together as a feature. Each feature can therefore represent multiple individual genetic anomalies and thus the full set of anomalies can be recast in terms of these features and further analysis performed on these directly.

The method of any one of the preceding claims, comprising identifying further genomic aberrations present in the sample; and classifying the cancer patient: in a first prognostic group based on the presence of one or more genomic aberrations selected from table 1 and in a second prognostic group based on the presence of one or more genomic aberrations selected from table 2. It will be appreciated that the absence of some or all of the genomic aberrations in table 1 may also be indicative of the second prognostic group. Similarly, the absence of some or all of the genomic aberrations in table 2 may also be indicative of the first prognostic group. The classification may thus combine the ARBS score and the presence of one or more genomic aberrations to determine the most likely prognostic group. The probability of the classification being correct may also be output with the classification.

TABLE 1
Genomic aberrations associated with alternative
cancer evolutionary type (evotype)

| Chromosome region or gene | Aberration |
|---|---|
| 1q42.12-1q42.13 | Loss of heterozygosity |
| 2q14.3-2q23.3 | Loss of heterozygosity |
| 5q11.1-5q23.1 (IL6ST, PDE4D) | Loss of heterozygosity |
| 5q15-5q23.1 (CHD1) | Loss of heterozygosity |
| 6q12-6q22.32 (MAP3K7, ZNF292) | Loss of heterozygosity |
| 13q12.3-13q21.1 (BRCA2, RB1) | Loss of heterozygosity |
| 13q13.3-13q33.1 (EDNRB) | Loss of heterozygosity |
| 3q21.2-3q29 | Gain |
| Chromosome 7 | Gain |
| 8p23.3-8p22 | Gain |
| 8q (MYC) | Gain |
| SPOP | Mutation |
| Kataegis | Present |
| Chromothripsis | Present |
| PGA clonal | Present |

TABLE 2
Genomic aberrations associated with canonical cancer evotype

| Chromosome region or (gene) | Aberration |
|---|---|
| 17p (TP53) | Loss of heterozygosity |
| 19p13.3-19p13.2 | Loss of heterozygosity |
| 21q22.2-21q22.3 (ERG) | Loss of heterozygosity |
| ETS | Gene fusion |
| Inter/intra chromosomal breakpoint ratio | High |

The number of breakpoints and/or ARBS score is omitted from Tables 1 and 2 above which focus on additional genomic aberrations. It will be appreciated that combinations including two, three, four or more of the genomic aberrations may be used in the classifying steps. The genomic aberrations may be selected based on their significance to the classification and/or the ease with which they can be identified within a sample. For example, genomic aberrations within targeted regions may be included in such combinations in preference to genomic aberrations which require whole genome testing.

The method may further comprise identifying further genomic aberrations in the biological sample selected from the group comprising loss of heterozygosity in one or more of the following regions: 2q14.3-2q23.3, 5q15-5q23, 5q11.1-5q14.1 (IL6ST, PDE4D), 6q12-6q22.32 (MAP3K7, ZNF292), 10q23.1-10q25, 16q12.1-16q24.3, 17p, 18q, gain of heterozygosity in one or more of the following regions: 3q21.2-3q29, whole chromosome 7, 8p23.3-8p22, 8q, 9q12.9-9q21.11 and whole chromosome 19, ratio of intra- to inter-chromosomal chained structural variants, kataegis, ETS, Percentage Genome Altered (subclonal component) and Percentage Genome Altered (clonal component). The subject may be classified in the first prognostic group based on the presence of one or more genomic aberrations selected from loss of heterozygosity in one or more of the following regions: 2q14.3-2q23.3, 5q11.1-5q14.1 (IL6ST, PDE4D), 5q15-5q23, 20 6q12-6q22.32 (MAP3K7, ZNF292), 18q, gain of heterozygosity in one or more of the following regions: 3q21.2-3q29, whole chromosome 7, 8p23.3-8p22, 8q, 9q12.9-9q21.11, kataegis, more particularly based on the presence of a combination of genomic aberrations selected from loss of heterozygosity in the following regions: 2q14.3-2q23.3, 6q12-6q22.32 (MAP3K7, ZNF292), 18q, and gain of heterozygosity in one or more of the following regions: whole chromosome 7 and 8q. The subject may be classified in the second prognostic group based on the presence of one or more genomic aberrations selected from the group comprising loss of heterozygosity in one or more of the following regions: 10q23.1-10q25, 16q12.1-16q24.3, 17p, gain of heterozygosity in one or more of the following regions: whole chromosome 19, ratio of intra- to inter-chromosomal chained structural variants, ETS, Percentage Genome Altered (subclonal component) and Percentage Genome Altered (clonal component), more particularly based on the presence of a combination of genomic aberrations selected from ratio of intra- to inter-chromosomal chained structural variants, loss of heterozygosity in one or more of the following regions: 10q23.1-10q25, 17p, ETS and Percentage Genome Altered (subclonal component).

For ease of reference, the features are listed in the tables below and are ranked in order of importance or significance of the feature to the classification. Thus, the classification may be based on a combination of the most important features, e.g. the top five, or even top three.

TABLE A
Features for depleted classification
indicative of first prognostic group
Features

Loss of heterozygosity: 2q14.3-2q23.3
Loss of heterozygosity: 6q12-6q22.32 (MAP3K7, ZNF292)
Loss of heterozygosity: 18q
Gain of whole chromosome 7
Gain: 8q
Loss of heterozygosity: 5q15-5q23
Loss of heterozygosity: 5q11.1-5q14.1 (IL6ST, PDE4D)
Gain: 8p23.3-8p22
Gain: 3q21.2-3q29
Kataegis
Gain: 9q12.9-9q21.11
Chromothripsis
SPOP
LOH: 12p12.32-12p12.3
LOH: 1p31.1-1p22.3
LOH: 1q42.12.1-1q42.13

TABLE B
Features for enriched classification
indicative of second prognostic group:
Features

Ratio of intra- to inter- chromosomal chained structural variants
Loss of heterozygosity: 10q23.1-10q25
Loss of heterozygosity: 17p
ETS
Percentage Genome Altered (subclonal component)
Percentage Genome Altered (clonal component)
Gain of whole chromosome 19
Loss of heterozygosity: 16q12.1-16q24.3

In the above Tables, the aberration may occur within any region of the chromosome that is referred to. In some instances, specific genes are provided in brackets; this refers to a gene/genes present in the chromosome region wherein the aberration may occur, but the aberration is not limited to occurring within this gene/genes.

A probability may be returned with each identified genomic aberration, including the ARBS score, to indicate the probability that the tumour belongs to the first or second prognostic groups (i.e. to either evotype) based on each identified genomic aberration. It will be appreciated that the features listed for the first prognostic group are strongly negative for the second prognostic group and vice versa. The classification may be based on a consideration of all features, including the features which are strongly indicative of a particular classification when present and the features which are strongly indicative that it is not that particular classification when present A probability threshold may be used to assign a classification, for example, when the probability is above p=0.5. It will be appreciated that the presence of an individual genetic alteration provides a smaller change in the probability of converging to a particular evotype than when the combination is present. Accordingly, a probability may be returned with the overall classification and the probability may be calculated based on the combination of genomic aberrations which have been identified.

Previously collected samples having known clinical outcomes may be analysed and the samples may be clustered into two or more clusters based on combinations of features which are indicative of the prognostic groups. A new sample may then be classified into a prognostic group based on the presence of features corresponding to the features of the clustered samples which are indicative of the prognostic group. This method of stratifying may thus be considered to be using a clustering classification (e.g. hierarchical clustering) and the classifications may be selected from meta-cluster A or meta-cluster B. A classification as meta-cluster A may be indicative of the first prognostic group, i.e. of the Alternative evotype. A classification as meta-cluster B may be indicative of the second prognostic group, i.e. of the Canonical evotype. Meta-cluster B may be sub-divided into two subclasses, meta-cluster B1 and meta-cluster B2.

The individual features which are characteristic of meta-cluster A (i.e. of the Alternative evotype) may include a combination of genetic aberrations including intra-chromosomal structural variants (SVs), SPOP mutations, chromothripsis and loss of heterozygosity in regions 5q15-5q23.1 (spanning CHD1) and 6q14.1-6q22.32 (MAP3K7, ZNF292). A tumour may be classified as meta-cluster A when at least some of the aberrations in this group (also known as a cluster) are present. The features which are characteristic of meta-cluster B1 (i.e. of the Canonical evotype) may include a combination of genetic aberrations including ETS gene fusions and loss of heterozygosity in regions 17p (TP53) and 19p13.3-19p13.2 and 22q11.21-22q11.22. A tumour may be classified as meta-cluster B1 when at least some of these aberrations are present. The features which are characteristic of meta-cluster B2 (i.e. of the Canonical evotype) may include a combination of genetic aberrations including ETS gene fusions, inter-chromosomal chained structural variants (cSVs) and loss of heterozygosity in regions 5q11.1-5q14.1 (IL6ST, PDE4D), 10q23.1-10q25.1 (PTEN) and 17p (TP53). A tumour may be classified as meta-cluster B2 when at least some of these aberrations are present.

For ease of reference, the features are listed in the tables below and are ranked in order of importance of the feature to the classification. Thus, the classification may be based on a combination of the most important features, e.g. the top five, or even top three.

TABLE C
Features for clustering
classification - Metacluster A
Features

Loss of heterozygosity: 5q11.1-5q14.1(IL6ST, PDE4D)
Loss of heterozygosity: 5q15-5q23 (CHD1)
Kataegis
Percentage Genome Altered (clonal component)
Loss of heterozygosity: 2q14.3-2q23.3
Loss of heterozygosity: 6q12-6q22.32 (MAP3K7, ZNF292)
Gain of whole chromosome 7
Loss of heterozygosity: 1q42.12.1-1q42.13
Loss of heterozygosity: 12p12.32-12p12.3
Loss of heterozygosity: 18q
Gain: 8q (MYC)
SPOP

TABLE D
Features for clustering
classification - Metacluster B
Features

Ratio of intra- to inter- chromosomal chained structural
variants
ETS
Percentage Genome Altered (subclonal component)
Loss of heterozygosity: 17p
Loss of heterozygosity: 16q12.1-16q24.3
Gain: 9q12.9-9q21.11
Gain of whole chromosome 19
Loss of heterozygosity: 21q22.2-21q22.3

This clustering classification may be used together with or separately from the ARBS classification to stratify patients. Thus, according to another aspect of the invention, there may be provided a method for stratifying a cancer patient into one of two prognostic groups, wherein the method comprises analysing a biological sample obtained from a subject with cancer using DNA sequencing, identifying genomic aberrations in the biological sample, and classifying, using a clustering classification, the cancer patient in a first prognostic group based on the presence of one or more genetic aberrations selected from a set of genetic aberrations including intra-chromosomal structural variants, SPOP mutations, chromothripsis and loss of heterozygosity in regions 5q15-5q23.1 (spanning CHD1) and 6q14.1-6q22.32 (MAP3K7, ZNF292). The cancer patient may be classified in a second prognostic group based on the presence of one or more genetic aberrations selected from a first set of genetic aberrations which includes ETS gene fusions and loss of heterozygosity (LOH) in regions 17p (TP53) and 19p13.3-19p13.2 and 22q11.21-22q11.22 or a second set of genetic aberrations which includes a combination of ETS fusions and inter-chromosomal chained structural variants (cSVs), as well as LOH affecting 17p (TP53), 10q23.1-10q25.1 (PTEN) and 5q11.1-5q14.1 (IL6ST, PDE4D).

The method may further comprise a step of determining the order in which the genomic aberrations occur. This may include performing bulk cell sequencing and determining the proportion of cells comprising each genetic aberration. The aberrations present in a higher proportion of cells are determined to have occurred prior to the aberrations present in a lower proportion of cells. The ordering may be a consensus ordering which may be determined using statistical ranking methods, such as Plackett-Luce model. The cancer patient may be classified based on the identified order and such a classification may be termed an ordering classification. Thus according to another aspect of the invention, there is provided a method for stratifying a cancer patient into one of two prognostic groups, the method comprising providing a biological sample from a subject with prostate cancer, analysing the biological sample using bulk-cell DNA sequencing, identifying genomic aberrations present in the biological sample; determining the proportion of cells in which the genomic aberrations are present, identifying an order in which the genomic aberrations occurred by determining that the genomic aberrations which are present in a larger proportion of cells occurred before the genomic aberrations which are present in a smaller proportion of cells, and classifying the cancer patient in one of the first and second prognostic groups based on the identified order.

The ordering for the first prognostic group may be termed Ordering II and the ordering for the second prognostic group may be termed Ordering I. The genomic aberrations which may be indicative of the ordering classifications include some or all of loss of heterozygosity in one or more of the regions 5q15-5q23.1 (spanning CHD1), 6q14.1-6q22.32 (MAP3K7, ZNF292), 8p (NKX3.1), 10q23.1-10q25.1 (PTEN), 13q12.3-13q21.1 (RB1, BRCA2) and 13q21.1-13q33.1 (EDNRB), 16q12.1-16q24.1 (CDH1) and 17p (TP53), SPOP mutations and ETS fusions. The cancer patient may be classified based on the identified order and such a classification may be termed an ordering classification. In other words, the accumulation of genetic aberrations may provide further insight into the evolutionary trajectory of the cancer, for example whether the cancer will converge to the alternative or the Canonical evotype (i.e. ordering II or ordering I respectively). As an alternative (or in addition) to identifying an order in which the genomic aberrations occurred based on the proportion of cells in which the genomic aberrations are present, the method may comprise analysing a second biological sample obtained at a subsequent time from the cancer patient using DNA sequencing, identifying genomic aberrations present in the second sample and comparing the genomic aberrations identified in the second sample with the genomic aberrations identified in the first sample to identify an order in which the genomic aberration occurred. Additional subsequent samples may be obtained at multiple time intervals and analysed to provide more information on the order in which the genomic aberrations occur. As such the present method may be used in a method of monitoring disease progression, selecting a therapy for cancer treatment or a method of therapy monitoring over time.

This ordering classification may be used together with or separately from the ARBS classification and/or the clustering classification to stratify patients.

The cancer patient may be classified as ordering II when loss of heterozygosity in one or more of the regions 6q14.1-6q22.32 (MAP3K7, ZNF292), 13q12.3-13q21.1 (RB1, BRCA2) and 13q21.1-13q33.1 (EDNRB) occur early in the order of genomic aberrations. Loss of heterozygosity in 5q15-5q23.1 (spanning CHD1) and SPOP mutations may also occur early in the order of genomic aberrations for cancers to be classified as ordering II. More frequent copy number gains may also be indicative of classification as ordering II. A late gain for chromosome 19 may also be indicative of classification as ordering II.

The cancer patient may be classified as ordering I when loss of heterozygosity in the region 8p (NKX3.1) or ETS fusion occurs early in the order of genomic aberrations. Loss of heterozygosity in one or more of the regions 10q23.1-10q25.1 (PTEN), 13q12.3-13q21.1 (RB1, BRCA2), 16q12.1-16q24.1 (CDH1) and 17p (TP53) occurring later in the order of genomic aberrations than loss of heterozygosity in the region 8p (NKX3.1) or ETS fusions may further be indicative of ordering I. A late gain for chromosome 19 (i.e. only present in later samples or occurring in a small proportion of cells in a single sample) may also be indicative of classification as ordering I. Occasionally, a very early loss of heterozygosity in the region 1q42.12-42.3 may be indicative of ordering I.

For ease of reference, the features which are most relevant to the Orderings classification are listed in the tables below and are ranked in order of importance of the feature to the classification. Thus, the classification may be based on a combination of the most important features, e.g. the top five, or even top three.

TABLE E
Features for Ordering II classification which
is indicative of first prognostic group
Features

Loss of heterozygosity: 6q12-6q22.32
Loss of heterozygosity: 5q15-5q23
Percentage Genome Altered (subclonal component)
Loss of heterozygosity: 13q21.1-13q33.1
Loss of heterozygosity: 13q12.3-13q21.1
Gain: 8p23.3-8p22
Ratio of intra- to inter- chromosomal chained structural variants
Gain: 9q12.9-9q21.11

TABLE F
Features for Ordering I classification which
is indicative of second prognostic group
Features

Loss of heterozygosity: 21q22.2-21q22.3
Loss of heterozygosity: 16q12.1-16q24.3
Loss of heterozygosity: 17p
Loss of heterozygosity: 8p
Loss of heterozygosity: 10q23.1-10q25
ETS

The three classifications may be applied separately or in combination to stratify the patient into one of the two prognostic groups. When there is a combination of more than one classification selected from the group of ARBS classification, clustering classification and ordering classification, the selected classifications may be considered to be intermediate classifications. An overall classification may be determined based on the intermediate classifications. For example, when all three intermediate classifications are used, the overall classification as the first prognostic group (Alternative evotype) may be provided when at least two of the intermediate classifications classify the patient in the first prognostic group. In other words, the tumour has at least two intermediate classifications selected from classification as a metacluster MC-A, an ARBS classification of depleted and an orderings classification of Ordering-II. A tumour may be assigned to the second prognostic group (Canonical evotype) based on a similar majority-vote approach when at least two of the classifications are indicative of the Canonical evotype, i.e. classify the patient in the second prognostic group. For example, a clustering classification as a metacluster MC-B1 or B2, an ARBS classification of enriched or indeterminate and an orderings classification of Ordering-I are indicative of an overall classification as a Canonical evotype.

Thus, according to another aspect of the invention, there may be provided a method for stratifying a cancer patient into one or two prognostic groups, wherein the method comprises; analysing a biological sample obtained from a subject with cancer or metastatic disease using bulk cell sequencing and DNA and/or RNA sequencing, determining, in the biological sample, locations of double stranded DNA breakpoints relative to androgen receptor binding sites (ARBS), identifying genomic aberrations in the biological sample, and determining the proportion of cells in the biological sample having one or more genetic aberrations selected from loss of heterozygosity in one or more of the regions 5q15-5q23.1 (spanning CHD1), 6q14.1-6q22.32 (MAP3K7, ZNF292), 8p (NKX3.1), 10q23.1-10q25.1 (PTEN), 13q12.3-13q21.1 (RB1, BRCA2) and 13q21.1-13q33.1 (EDNRB), 16q12.1-16q24.1 (CDH1) and 17p (TP53), SPOP mutations and ETS fusions. The method comprises obtaining a first intermediate classification, using an ARBS classification, wherein the cancer patient is classified: in a first prognostic group when the determined locations are less frequently proximal to androgen receptor binding sites than expected and in a second prognostic group when the determined locations are more frequently proximal to androgen receptor binding sites than expected. The method comprises obtaining a second intermediate classification, using a clustering classification, wherein the cancer patient is classified in the first prognostic group based on the presence of one or more genetic aberrations selected from a set of genetic aberrations including intra-chromosomal structural variants, SPOP mutations, chromothripsis and loss of heterozygosity in regions 5q15-5q23.1 (spanning CHD1) and 6q14.1-6q22.32 (MAP3K7, ZNF292) and in the second prognostic group based on the presence of one or more genetic aberrations selected from a first set of genetic aberrations which includes ETS gene fusions and loss of heterozygosity in regions 17p (TP53) and 19p13.3-19p13.2 and 22q11.21-22q11.22 or a second set of genetic aberrations which includes ETS gene fusions, inter-chromosomal chained structural variants and loss of heterozygosity in regions 5q11.1-5q14.1 (IL6ST, PDE4D), 10q23.1-10q25.1 (PTEN) and 17p (TP53). The method comprises obtaining a third intermediate classification using an orderings classification wherein the cancer patient is classified in one of the first and second prognostic groups based on an identified order in which the genomic aberrations occurred, wherein identifying the order in which the genomic aberrations occurred comprises determining that the genomic aberrations which are present in a larger proportion of cells occurred before the genomic aberrations which are present in a smaller proportion of cells. The method comprises determining an overall classification as the first prognostic group when at least two of the first, second and third intermediate classifications classify the patient in the first prognostic group and as the second prognostic group when at least two of the first, second and third intermediate classifications classify the patient in the second prognostic group.

The path to the Alternative-evotype may result from the following sequence of steps: certain genetic alterations cause altered AR binding, this promotes DNA breaks in a different set of positions, this leads to different copy number changes and gives rise to a mechanistically different form of the disease. By contrast, Canonical-evotype tumours progress down the ‘default route’ and display genetic alterations with breakpoints near normal AR binding sites. These accumulate to such a degree that the alternative route is closed (maybe the cell is unviable or progress on the path to the alternative route is now too far). Accordingly, the alterations to AR binding may be considered important to determining the classification as Canonical-evotype or Alternative-evotype. It will be appreciated that there are other ramifications of this AR cistrome modification that could be used to test for evotype (such as the occurrence of point mutations in open chromatin regions associated with Alternative-AR binding).

The probability of the classification being correct may also be output with the classification. For example, when an SPOP mutation occurs first, it confers high probability (˜0.91) of progression to the Alternative-evotype. As described above, other routes to the Alternative-evotype involve the accumulation of multiple individual LOH events involving genes such as MAP3K7, CHD1 or EDNRB in any order. LOH of IL6ST or gain of region 8p23.3-8p22 strongly influence convergence to Alternative-evotype after a number of aberrations had already accumulated. Conversely, classification as the second prognostic group, e.g. Canonical-evotype, may have a higher probability when a few key aberrations are identified, for example: early TP53 loss or ETS gene fusion almost certainly ensures fixation to the Canonical-evotype. Loss of regions covering PTEN or CDH1 may also be indicative of the Canonical-evotype. For the Canonical evotype, there were a number of aberrations that occurred late but ensured convergence, and therefore were often the last step, particularly LOH of 19p13.3-19p13.2, and gains of chromosome 19 and region 22q11.1-22q11.23.

The various classifications above are based on the presence (or absence of) genomic aberrations. As an alternative to considering each classification separately, the subject may be classified in one of the prognostic groups based on the identified genomic aberrations. The probability of the classification being correct may also be output with the classification. The genomic aberrations are ranked in order of importance according to their significance determined by the proportion of tumours with this features:

TABLE 1
Genomic aberrations positively associated
with Alternative cancer evolutionary type

| Genomic aberration | Type of aberration |
|---|---|
| 6q12-6q22.32 (MAP3K7, ZNF292) | Loss of heterozygosity |
| 13q12.3-13q21.1 (BRCA2, RB1) | Loss of heterozygosity |
| 13q21.1-13q33.1 (EDNRB) | Loss of heterozygosity |
| PGA clonal | High |
| Chromosome 7 | Gain |
| Kataegis | Present |
| 5q15-5q23.1 (CHD1) | Loss of heterozygosity |
| 8q (MYC) | Gain |
| 8p23.3-8p22 | Gain |
| 5q11.1-5q14.1 (IL6ST, PDE4D) | Loss of heterozygosity |
| 2q14.3-2q23.3 | Loss of heterozygosity |
| Chromothripsis | Present |
| 3q21.2-3q29 | Gain |
| 1q42.12-1q42.13 | Loss of heterozygosity |
| SPOP | Mutation |

It is noted that the ARBS score is not positively associated with the first prognostic group. However, a low ARBS score is highly indicative of the first prognostic group and may thus be used in combination with the presence of the listed genomic aberrations.

TABLE 2
Genetic aberrations positively associated
with Canonical cancer evotype

| Genomic aberration | Type of aberration |
|---|---|
| ETS | Gene fusion |
| Inter/intra chromosomal breakpoint ratio | High |
| 21q22.2-21q22.3 (ERG) | Loss of heterozygosity |
| 17p (TP53) | Loss of heterozygosity |
| 19p13.3-19p13.2(LKB1) | Loss of heterozygosity |

The classification may be based on a combination of the most important features, e.g. the top five, or even top three. Alternatively, the most important features may be selected by ranking the features based on their importance in the subclassifications. For example, a feature which is important to all three subclassifications may preferably be included when considering a combination. Features which are important to two subclassifications may optionally be included. A consideration of the time and effort involved in conducting the test to see if the feature is present may also influence whether or not to include the feature in the genomic aberrations to be identified before making the classification. The features which reflect changes in specific regions of the chromosome may thus be selected for the classification.

The genomic aberrations may be identified using known methods. For example, structural variants may be detected using Brass (31). Somatic mutations in the SPOP gene may be determined using CaVEMan. Copy number alterations (i.e. LOH, HD and Gains) may be determined using the Battenberg algorithm with whole genome sequencing, or through ADTEx, CONVEX SeqCNV with exome or targeted sequencing. ETS fusions may be detected using BRASS. Chromothripsis may be identified by identifying copy number breakpoints and segmenting inter-breakpoint distance along the genome using piecewise constant fitting (pcf from the R package copynumber v1.22.0). Regions with a density higher than 1 breakpoint per 3 Mb may be flagged as high-density regions. A chromothripsis region may be defined as a high-density region with a number of copy number breakpoints N>15; a non-random segment size distribution (Kolmogorov-Smirnov test against the exponential distribution, P<0.05); at most three allele-specific copy number states covering more than min (1, 0.006N+1.1) fraction of the region; and the proportion of each type of structural variant is random with equal probability PTD=PDel=PH2Hi=PT2Ti=0.25 (multinomial test P>0.01), where TD=tandem duplication, Del=deletion, H2Hi=head-to-head inversion and T2Ti=tail-to-tail inversion.

For example, kataegis may be identified using SeqKat https://github.com/cran/SeqKat. DNA Breakpoints associated with chained events may be identified using Chainfinder (http://archive.broadinstitute.org/cancer/cga/chainfinder) version 1.01. Clonal/subclonal ratio can be used to quantify the number of SNVs that are in all cancer cells in the sample (clonal) or only in a subset (subclonal) i.e. SNVs with cancer cell fraction (CCF)=1 and CCF<1 respectively. Percentage genome altered may be calculated as the percent total of the genome that is affected by CNAs (copy number alterations) (37). We also recorded the percentage affected by clonal and subclonal CNAs (i.e. CNAs with CCF=1 and CCF<1 respectively). The “number of breakpoints” is the number of DNA breakpoints which can be determined by BRASS. Inter/intra chromosomal breakpoints can be determined using Chainfinder to identify breakpoints that occurred as part of the same event (e.g. chromosomes co-locate at a transcription factory, break up, get put back together wrongly). If these events involve different i.e. non-homologous chromosomes (e.g. interchromosomal translocation) then they are classed as inter-chromosomal breakpoints, and if they only involve the same chromosome (e.g. deletion) then they are classified as intra-chromosomal. The ratio between the number of breakpoints in these two categories can then be determined. “Gene fusion” refers to a hybrid gene formed from two previously separate genes. Multiple genetic events may lead to gene fusions occurring such as gene translocations, deletions etc.

The term “loss of heterozygosity” may refer to a chromosomal event wherein a gene or chromosome region is lost, and is a common form of allelic imbalance by which a heterozygous somatic cell becomes homozygous because one of the two alleles gets lost. Loss of heterozygosity may lead to the somatic loss of wild-type alleles; this form of chromosome instability is sufficient to provide selective growth advantage and has been recognized as a major cause of tumorigenesis. It will be appreciated that the term “gain” is used to indicate that a region of a chromosome or full chromosome arm has been duplications one or more times.

Gene loss can occur through many mechanisms, including large-scale deletions; more often it is the result of nonsense mutations or frameshifts. The former causes a premature stop codon and is the result of a standard mutation from one nucleotide to another. Frameshift mutations are the result of insertions or deletions, not a multiple of three and usually small, within the coding region that change how the codons are translated into amino acids. These processes can occur and result in the loss of protein-coding genes. However similar effects can be achieved for the loss of noncoding genes or regulatory regions.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

DNA and/or RNA sequencing is used for the analysis of the present samples. Next generation sequencing, high-throughput sequencing methods may be used. Whole genome sequencing, whole exome sequencing, targeted gene sequencing, RNA-seq, transcriptome sequencing, methylation sequencing, bisulphite sequencing or a combination thereof may be performed. Bulk cell sequencing and/or single cell sequencing methods may be used. Methods used for DNA and RNA extraction are known in the art and can be used to obtain DNA or RNA from various samples in order to perform sequencing methods.

The cancer may be prostate cancer.

The biological sample may be a biopsy from tumour or a whole blood sample. The biological sample may be obtained from a radical prostatectomy or biopsy performed on a subject or during transurethral resection. The sample may be fresh-frozen, or formalin fixed.

The sample may be from a subject with acinar adenocarcinoma, ductal adenocarcinoma, transitional cell carcinoma (urothelial carcinoma), squamous cell prostate cancer, small cell prostate cancer, large cell prostate cancer, mucinous adenocarcinoma, signet cell prostate cancer, basal cell prostate cancer, leiomyosarcoma or rhabdomyosarcoma.

### Complementary Methods

As set out above, the first prognostic group may be termed an Alternative evotype and the second prognostic group may be termed a Canonical evotype. The Alternative evotype is associated with a poorer prognosis than the Canonical evotype. Accordingly, the methods of stratifying the cancer patient into one of two prognostic groups described above may also be used to predict the clinical outcome of a patient.

The method above may comprise the additional step of treating the patient with a therapy. Alternatively, there may be provided as another aspect of the invention, a method of treating cancer in a subject comprising stratifying a subject into one of two prognostic groups according to the methods described above and further comprising administering a cancer therapy to the subject.

There are a number of therapies which are recommended for the treatment of prostate cancer. Treatments are recommended depending on the stage of disease progression. Radiotherapy, hormone treatment and chemotherapy are the three options that are often used in prostate cancer treatment. A single treatment or a combination of treatments may be used.

Chemotherapy is often used to treat prostate cancer that has invaded to other organs of the body (metastatic prostate cancer). Chemotherapy destroys cancer cells by interfering with the way they multiply. Chemotherapy may be used to control prostate cancer and reduce symptoms; therefore, daily life is less affected. Patients may receive hormone therapy before undergoing chemotherapy to increase the chance of successful treatment.

Radiotherapy may be used to treat localised and locally-advanced prostate cancer. Radiotherapy can also be used to slow the progression of metastatic prostate cancer and relieve symptoms. Hormone therapy may also be recommended after radiotherapy to reduce the chances of relapsing.

Hormone therapy is often used in combination with radiotherapy. Hormone therapy alone should not normally be used to treat localised prostate cancer in men who are fit and willing to receive surgery or radiotherapy. Hormone therapy can be used to slow the progression of advanced prostate cancer and relieve symptoms. Hormones control the growth of cells in the prostate. In particular, prostate cancer needs the hormone testosterone to grow. The purpose of hormone therapy is to block the effects of testosterone, either by stopping its production or by stopping a patient's body from using testosterone.

Other treatments that may be used in prostate cancer therapy include surgery, e.g. radical prostatectomy, high intensity focused ultrasound therapy, cryotherapy, brachytherapy, patient monitoring, trans-urethral resection of the prostate, gene therapy, viral therapy, RNA therapy bone marrow transplantation, nanotherapy, targeted anti-cancer therapies or oncolytic drugs. Examples of therapeutic agents include steroids, antibodies targeting prostate specific membrane antigen (PSMA), checkpoint inhibitors, antineoplastic agents, immunogenic agents, attenuated cancerous cells, tumour antigens, antigen presenting cells such as dendritic cells pulsed with tumour-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumour-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), or steroids.

The present method allows clinicians to select appropriate treatment for prostate cancer based on the evotype (i.e. based on the prognostic group). For example, the Alternative evotype is associated with poor prognosis and so a more aggressive treatment may be selected. Aggressive treatment may be selected from one or more of external beam radiation, brachytherapy, radical prostatectomy, hormone therapy, and/or chemotherapy. In particular external beam radiation and brachytherapy may be combined. The Canonical evotype is associated with standard prognosis and so standard therapy may be adopted involving patient monitoring, radiotherapy, hormone treatment, chemotherapy and/or a combination thereof.

The progression of prostate cancer may be determined using the methods of the present invention. The therapeutic efficacy of a prostate cancer treatment may be monitored using the methods of the present invention.

### Kits

A kit for use in the method of the present invention is provided herein.

The kit may comprise reagents for performing DNA and/or RNA sequencing and a tool for the detection of DNA double stranded breaks. The reagents for DNA and/or RNA sequencing may be for whole genome sequencing, whole exome sequencing, targeted gene sequencing, RNA-seq, transcriptome sequencing, methylation sequencing, bisulphite sequencing or a combination thereof. Double stranded breaks may be identified by probes such as fluorescent nucleotides or antibodies. Double stranded DNA breaks may also be identified in the sequencing data using computational tools such as BRASS.

The kit may also comprise instructions for use.

The kit may also comprise a probe for detection of one or more of the genomic aberrations listed in any one or Table 1, Table 2 or Tables A to F. These probes may be DNA probes for annealing to specific target sequences in sample DNA, for example FISH (Fluorescence in situ hybridization) may be used.

The kit may comprise one or more probes for detection of one or more of the genomic aberrations selected from: loss of heterozygosity in regions 17p (TP53), 19p13.3-19p13.2, and 21q22.2-21q22.3 (ERG) and instructions for use. Additionally or alternatively, the kit may comprise one or more probes for the detection of one or more of the genomic aberrations selected from loss of heterozygosity in any one of the regions: 1q42.12-1q42.13, 2q14.3-2q23.3, 5q11.1-5q14.1 (IL6ST, PDE4D), 5q15-5q23.1 (spanning CHD1), 6q14.1-6q22.32 (MAP3K7, ZNF292), 13q12.3-13q21.1 (RB1, BRCA2), 13q21.1-13q33.1 (EDNRB) and and/or gain in any one of the regions: 3q21.2-3q29, Chromosome 7, 8p23.3-8p22 and 8q (MYC).

The kit may further comprise a probe for detection of one or more of the genetic aberrations selected from; loss of heterozygosity in regions 1q42.12-1q42.13, 2q14.3-2q23.3, 5q11.1-5q23.1 (IL6ST, PDE4D), 5q15-5q23.1 (CHD1), 6q12-6q22.32 (MAP3K7, ZNF292), 13q12.3-13q21.1 (BRCA2, RB1), 13q13.3-13q33.1 (EDNRB), 17p (TP53), 19p13.3-19p13.2 (LKB), 21q22.2-21q22.3 (ERG), gain in regions 3q21.2-3q29, Chromosome 7, 8p23.3-8p22, 8q (MYC), intra-chromosomal structural variants, SPOP mutations, kataegis, chromothripsis, ETS gene fusions, PGA clonal, high number of breakpoints, high ratio of Inter/intra chromosomal breakpoint. In an embodiment the kit may further comprise a probe for detection of one or more of the genetic aberrations selected from intra-chromosomal structural variants, SPOP mutations, chromothripsis, ETS gene fusions, loss of heterozygosity in regions 5q15-5q23.1 (spanning CHD1), 6q14.1-6q22.32 (MAP3K7, ZNF292), 17p (TP53), 19p13.3-19p13.2, 22q11.21-22q11.22, 5q11.1-5q14.1 (IL6ST, PDE4D), and/or 10q23.1-10q25.1 (PTEN) and instructions for use. In an embodiment the kit comprises a probe for the detection of one or more of the genetic aberrations selected from 5q15-5q23.1 (spanning CHD1), 6q14.1-6q22.32 (MAP3K7, ZNF292), 8p (NKX3.1), 10q23.1-10q25.1 (PTEN), 13q12.3-13q21.1 (RB1, BRCA2), 13q21.1-13q33.1 (EDNRB), 16q12.1-16q24.1 (CDH1) and 17p (TP53), SPOP mutations, ETS fusions and instructions for use.

### BRIEF DESCRIPTION OF FIGURES

FIG. 1a is a schematic flowchart of the computer-implemented steps to discover Canonical or Alternative evotypes;

FIG. 1b illustrates the different types of data which are used as input data;

FIG. 2a is a schematic illustration of a neural network engine implementing a latent feature model which is used in the method of FIG. 1a;

FIG. 2b is a schematic graphical representation of a latent feature model;

FIG. 3 is a graph showing the frequency with which a particular number of features is estimated for 200 network runs with subsets of the data;

FIG. 4 is a schematic illustration of the steps taken when combining multiple weight matrices to arrive at the fixed weight matric of FIG. 2a;

FIGS. 5a and 5b are heatmaps showing the relationship between the input data and the patients and the reduced set of feature data and the patients respectively;

FIG. 6 is a dendrogram showing the probability of observing the listed features in each cluster together with a discrimination score quantifying the relevance of each feature in predicting relapse;

FIG. 7a plots the normalised proportion of DNA breakpoints for each sample ordered by the normalised proportion;

FIG. 7b is a heatmap of genomic features for each sample using the ordering from FIG. 7a;

FIG. 7c is a dendrogram showing the CNA proportion in each ARBS group;

FIG. 8 is a plot of BIC score against the number of mixture components showing the mean score as well as individual scores;

FIG. 9a plots the proportion of samples against the Plackett-Luce coefficient for the Ordering I and Ordering II;

FIG. 9b plots the copy number alterations against the Plackett-Luce coefficient for the Ordering I and Ordering II;

FIG. 10a plots the progression free survival against time for the patients having tumours classified as either evotype;

FIGS. 10b, 10c and 10d plots the proportion for each evotype at each tumour stage, each ISUP Gleason Grade Group and by PSA (ng/ml), respectively;

FIG. 10e is a bar chart showing the prevalence of each genetic aberration in each evotype;

FIG. 11a is a flowchart of a statistical algorithm for obtaining the probability of convergence to the Canonical or Alternative evotypes based on accumulation of genetic alterations;

FIG. 11b is a surface plot showing the probability density of a tumour being assigned to the Canonical evotype relative to the number of aberrations;

FIG. 12a is a 2D surface plot showing the probability density of all Canonical-evotype tumours being assigned to the Canonical-evotype as the number of aberrations increase;

FIG. 12b is a graph showing the proportion of lineages that converged to the Canonical-evotype at each number of genetic alterations;

FIG. 12c is a bar plot showing the relative proportion of genetic alterations and the position in which they occurred for the lineages which converged to the Canonical-evotype;

FIG. 13a is a 2D surface plot showing the probability density of all Alternative-evotype tumours being assigned to the Alternative-evotype as the number of aberrations increase;

FIG. 13b is a graph showing the proportion of lineages that converged to the Alternative-evotype at each number of genetic alterations;

FIG. 13c is a bar plot showing the relative proportion of genetic alterations and the position in which they occurred for the lineages which converged to the Alternative-evotype;

FIG. 14a shows the aberrations present in the Canonical-evotype tumours when split into ETS− and ETS+; and

FIG. 14b shows the Kaplan-Meier plot for ETS+ and ETS− tumours that were assigned to the Canonical-evotype;

FIG. 15a is a schematic flowchart of the computer-implemented steps to classify tumours as canonical or Alternative evotypes;

FIGS. 15b and 15c are plots of the relevance of features for classifying a tumour to a particular cluster: Metacluster A or Metacluster B respectively;

FIGS. 15d and e are plots of the relevance of features for classifying a tumour as Alternative (depleted tumour) or canonical evotype (enriched) tumour;

FIGS. 15f and g are plots of the relevance of features for classifying a tumour as a particular ordering: Ordering II or Orderings I respectively;

FIG. 15h is a plot of the relevance of features for classifying a tumour as a Canonical or Alternative evotype tumour directly;

FIG. 16 is a plot of the relevance of features for classifying a tumour as a Canonical or Alternative evotype tumour directly using RNA sequencing; and

FIG. 17 is a schematic of an associated system for performing the computer-implemented aspects of the methods.

### DESCRIPTION OF EMBODIMENTS

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of immunology, molecular biology, cell biology, chemistry, biochemistry and recombinant DNA technology, which are within the skill of the art. Such techniques are explained fully in the literature, see, e.g., Green and Sambrook et al., Molecular Cloning: A Laboratory Manual, 4th ed., Cold Spring Harbor Laboratory Press, Cold Spring Harbor, N.Y. (2012).

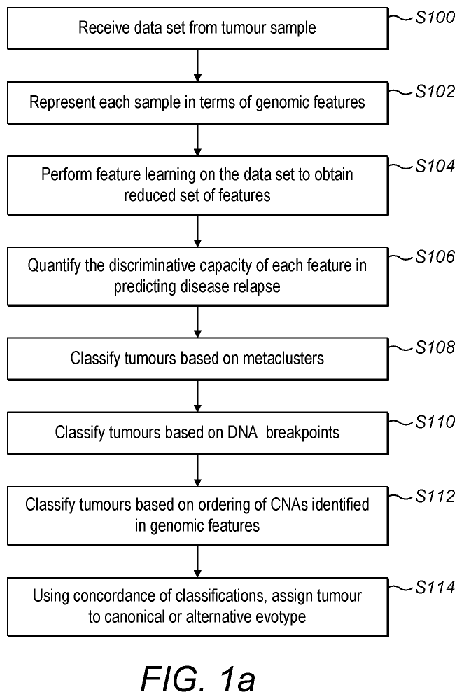

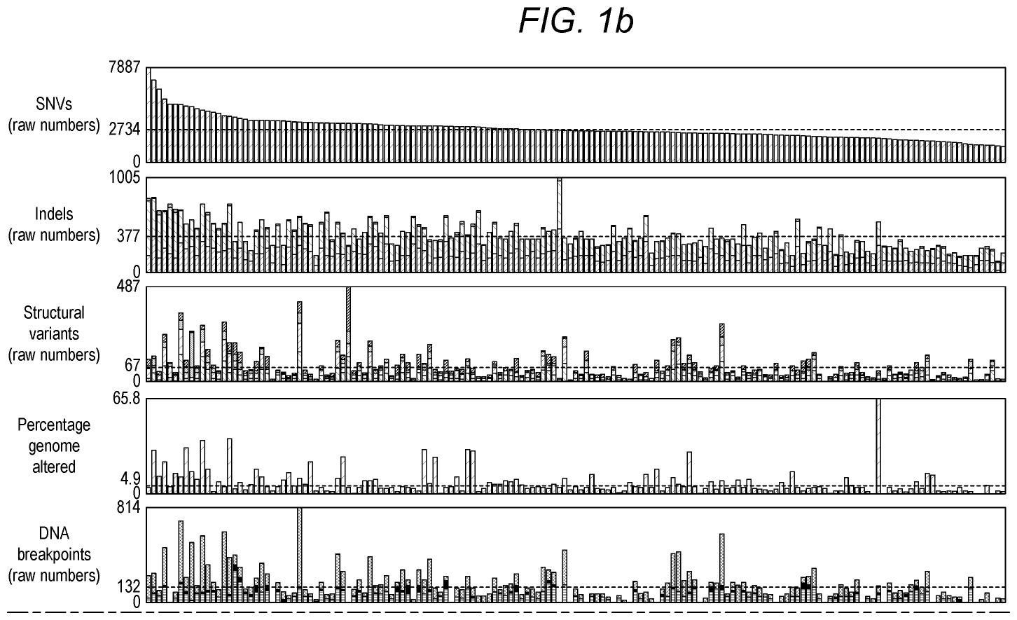

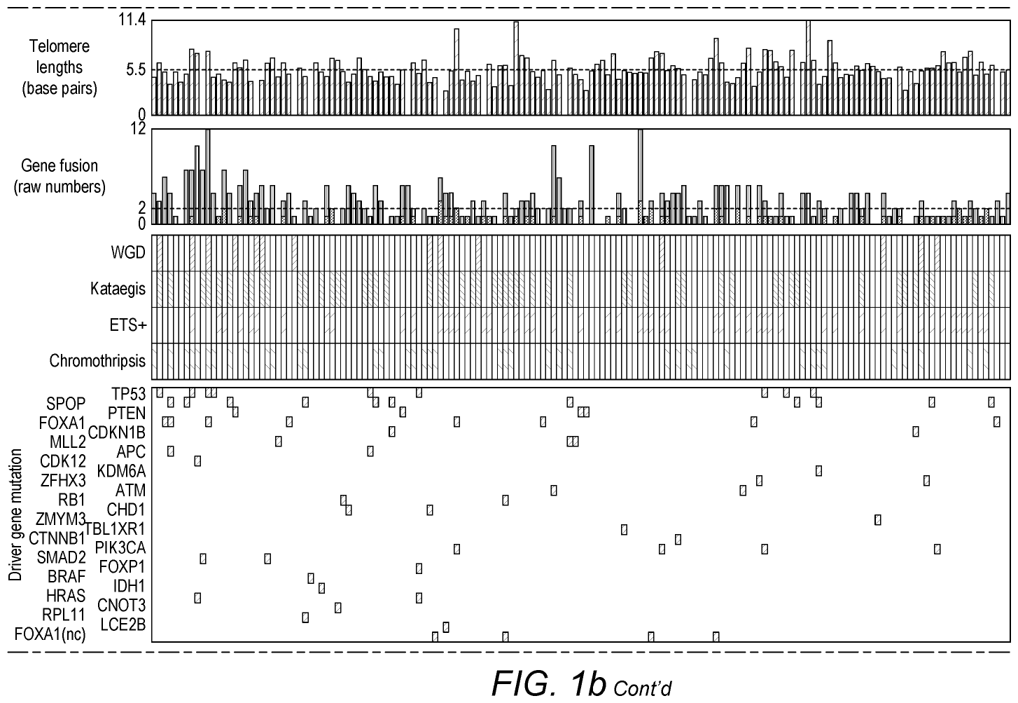

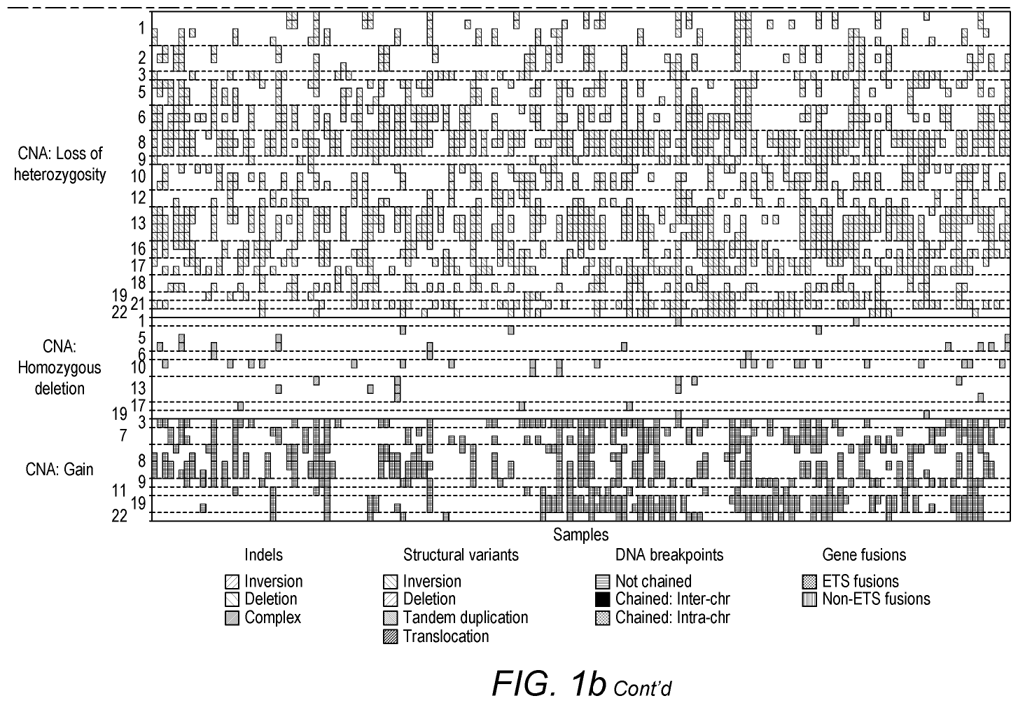

FIG. 1a is a schematic flowchart of the steps in the method to discover the Canonical and Alternative evotypes, which may be a computer-implemented method. As shown, the first step is to receive a data set of information collected from a patient's tumour sample (step S100). The data set may be collected by using DNA or RNA sequencing, for example in this case whole genome sequencing (target depth: 50λ) on the sample together with matched blood controls. The data set may comprise a large number (e.g. over 120, perhaps as many as 140) summary measurements or genomic features and each sample may be represented in terms of these features (step S102). These measurements may include some or all of the number of single nucleotide variants (SNVs), the number of indels (insertions, deletions or complex), the number of structural variants including genomic rearrangements (inversion, deletion, tandem duplication and translocation) and mutational signatures, the percentage of the genome altered (PGA), the DNA breakpoints (and whether they are involved in a chained intra- or inter-chromosomal structural variant), telomere lengths, the numbers of gene fusions, the presence or absence of any one of whole genome duplication (WGD), kataegis, ETS+ status and chromothripsis, the presence or absence of important driver mutations and copy number alterations (CNA; split into loss of heterozygosity (LOH), homozygous deletion (HD) and gains). The data may be collected using any known techniques, including the ones described below in relation to the data used to develop the classifications.

The next step S104 is one in which the input data is reformulated as a reduced set of features that encapsulate the underlying relationships between the original inputs. As explained in more detail below, this may be done using an adapted unsupervised neural network to perform feature learning on the data set, identifying associations between inputs to obtain a reduced set, e.g. having 30 features (shown in the table below).

Table of reduced set of features:

| Feature name (or | |
|---|---|
| chromosome | Inputs associated with each feature |
| region) | (number is the raw number) |
| Indels; PGA | Number of indels, number of deletions, PGA subclonal |
| subclonal | |
| PGA clonal; ploidy | PGA clonal, PGA total, ploidy |
| Kataegis | Kataegis |
| ETS gene fusion | ETS status, TMPRSS2: ERG fusion. Loss of heterozygosity of |
| | 21q22.2-21q22.3 |
| Intra-chromosomal | Number of SVs, Number of SV inversions, Number of SV deletions, |
| SVs | Number of SV tandem duplications, Number of SV translocations; |
| | Number of breakpoints, Number of chains, Number of Chained |
| | breakpoints, Number of breakpoints in longest chain, Number of |
| | deletion bridges, Number of Intra-chromosomal breakpoints |
| DNA breakpoint | Number of breakpoints, Number of chains, Number of Chained |
| burden | breakpoints, Number of breakpoints in longest chain, Number of |
| | deletion bridges, Number of Intra-chromosomal chained breakpoints, |
| | Number of Inter-chromosomal chained breakpoints, Mean |
| | breakpoints, Median breakpoints |
| Inter-chromosomal | Proportion of breakpoints in chains, Number of breakpoints in longest |
| SVs | chain, Max number of breakpoints per chain, Number of deletion |
| | bridges, Inter-chromosomal chained breakpoints, mean breakpoints |
| | per chain, median breakpoints per chain, mean chrs per chain, median |
| | chrs per chain, ration of inter/intra-chromosomal chained breakpoints |
| SPOP mutations | SPOP mutations |
| 1q31.1-1q22.3 | Loss of heterozygosity |
| 1q42.12-1q42.13 | Loss of heterozygosity |
| 2q14.3-2q23.3 | Loss of heterozygosity |
| 5q11.1-5q14.1 | Loss of heterozygosity |
| (IL6ST, PDE4D) | |
| 5q15-5q23.1 | Loss of heterozygosity |
| (CHD1) | |
| 6q12-6q22.32 | Loss of heterozygosity |
| (MAP3K7, | |
| ZNF292) | |
| 8p | Loss of heterozygosity |
| 10q23.1-10q25.1 | Loss of heterozygosity, HD |
| 13q12.3-13q21.1 | Loss of heterozygosity |
| (BRCA2, RB1) | |
| 13q21.1-13q33.1 | Loss of heterozygosity |
| (EDNRB) | |
| 16q12.1-16q24.3 | Loss of heterozygosity |
| 17p | Loss of heterozygosity |
| 18q | Loss of heterozygosity |
| 19p13.3-19p13.2; | Loss of heterozygosity |
| 22q11.21- | |
| 22q11.22 | |
| 3q21.2-3q.29 | Chromosomal gain or focal amplification |
| Chromosome 7 | Chromosomal gain or focal amplification |
| 8p23.3-8p22 | Chromosomal gain or focal amplification |
| 8q; PGA subclonal | Chromosomal gain or focal amplification |
| 9q12-9q21.11 | Chromosomal gain or focal amplification |
| 19; | Chromosomal gains or focal amplifications |
| 22q11.1-22q11.23 | |
| Chromothripsis | Proportion of genome affected by chromothripsis, number of distinct |
| | chromothripsis regions, max size of chromothripsis region |

With this approach the relationship between inputs and features is more easily interpretable, and we named features by the genomic aberrations to which they corresponded. A genomic aberration as used herein may thus be defined is any alteration of a genomic sequence (i.e. a genetic aberration), for example a deletion, insertion, inversion, duplication, loss of heterozygosity, DNA breakage, gene fusion, any other chromosomal mutation, or a measure of such genomic alterations, for example PGA (percentage genome altered), number of breakpoints, ARBS score. Where features reflect attributes of more than one genomic input, the attributes are separated by a semi-colon in the feature name. We represented each sample in terms of these genomic features, and this formed the basis for our discovery as described below. As set out at step S106, the next step is to quantify the discriminative capacity of each feature in predicting disease relapse to identify patterns of genomic aberrations indicative of adverse clinical outcome.

Using the information from step S106 together with the feature presentation as inputs to a two-stage clustering method led to the identification of two distinct metaclusters that were characterised by different sets of aberrations. Thus, as set out in step S108 and described in more detail below, tumours could be classified as belonging to Metacluster A (MC-A), Metacluster B1 (MC-B1) or Metacluster B2 (MC-B2). A tumour sample exhibiting a combination of intra-chromosomal structural variants (SVs), SPOP mutations, chromothripsis, and loss of heterozygosity (LOH) in regions 5q15-5q23.1 (spanning CHD1) and 6q14.1-6q22.32 (MAP3K7, ZNF292) may be classified as Metacluster A (MC-A). A tumour sample exhibiting a combination of ETS fusions, as well as LOH affecting 17p (TP53) and regions 19p13.3-13.2 and 22q11.21-22q11.22 is classified as Metacluster B1 (MC-B1). A tumour sample exhibiting a combination of ETS fusions and inter-chromosomal chained structural variants (cSVs), as well as LOH affecting 17p (TP53), 10q23.1-10q25.1 (PTEN) and 5q11.1-5q14.1 (IL6ST, PDE4D) is classified as Metacluster B2 (MC-B2).

The next step is to investigate the influence of Androgen Receptor (AR) on the DNA breakpoints involved. AR is known to precipitate DNA double strand breaks (DSB) in conjunction with topoisomerase II-beta, and AR-associated breakpoints are frequent in early-onset prostate cancer. As shown at step S110, tumours may be classified as enriched when breakpoints occurred significantly more often proximal to AR binding sites (ARBS) than expected, depleted when breakpoints occurred significantly less often proximal to AR binding sites (ARBS) than expected or indeterminate, if they displayed no statistically significant association. As explained in more detail below, investigating the ARBS groups in conjunction with the previously-identified features, depleted tumours were associated with multiple CNAs, chromothripsis and SPOP mutations. Enriched tumours were associated with CNAs affecting 16q12.1-16q24.3 (CDH1) and 17p (TP53), high inter/intra-chromosomal cSVs ratio, and ETS fusions. Further clustering work also confirms the association between these CNAs and ARBS-distal breakpoint prevalence.

The next step was to adapt a Plackett-Luce mixture model to extract the consensus ordering of the CNAs identified in the genomic features. Bayesian model selection determined that two separate ordering profiles were optimal. For the orderings classification, each tumour was classified as belonging to one of two Orderings-Ordering-I and Ordering-II (step S112). The two profiles displayed notable differences. A tumour classified as Ordering-I frequently experienced an early 8p LOH (spanning NKX3.1) and ETS fusions and a lack of LOH of regions covering the RB1, BRCA2, CDH1, TP53 or PTEN genes could also occur. A very early LOH of 1q42.12-42.3 was also possible for tumours in this Ordering. A tumour classified as Ordering-II often shows early LOH events covering MAP3K7 and 13q (EDNRB, RB1, BRCA2) and copy number gains. An early mutation of the SPOP gene and LOH covering CHD1 may also be present but is less common. Both orderings showed late gains of chromosome 19.

The concordance of these three classification methods revealed a remarkable relationship. We introduce the term evotype (evolutionary type) to describe tumours linked by common modes of evolution resulting in similar disease characteristics. Metacluster MC-A is largely a subset of the depleted group and both are almost entirely subsets of Ordering-II. We can therefore deduce that there exists a subset of tumours that exhibit all the corresponding properties: an evolutionary trajectory (Ordering-II), a breakpoint mechanism (ARBS classification of depleted) and characteristic patterns of aberrations (Metacluster: MC-A). The term evotype (evolutionary type) can be used to describe tumours linked by common modes of evolution resulting in similar disease characteristics. Tumours that are assigned to at least two of MC-A, depleted or Ordering II may be classified as an Alternative-evotype. Similarly, tumours that are assigned to at least two of a clustering classification as a metacluster MC-B1 or B2, an ARBS classification of enriched or indeterminate and an orderings classification of Ordering-I are indicative of an overall classification as a Canonical evotype. This concordance can be used to assign the tumour to one of the two evotypes (step S114).

### Patient Samples and Data Used in Developing Classifications

The clustering classification, the ARBS classification and the orderings classifications used above are based on applying three statistical and machine-learning methods to genomic measurements collected from 159 samples. The data was collected from cancer samples from 205 patients treated at the Royal Marsden NHS Foundation Trust, London, at the Addenbrooke's Hospital, Cambridge, at Oxford University Hospitals NHS Trust, and at Changhai Hospital, Shanghai, China, as described previously (31, 32). Ethical approval was obtained from the respective local ethics committees and from The Trent Multicentre Research Ethics Committee. All patients were consented to ICGC standards. 159 of the samples passed stringent quality control for copy number profiles and structural variants and were used in this study.

DNA from frozen tumour tissue and whole blood samples (matched controls) was extracted and quantified using a ds-DNA assay (UK-Quant-iT™ PicoGreen® dsDNA Assay Kit for DNA) following the manufacturer's instructions with a Fluorescence Microplate Reader (Biotek SynergyHT, Biotek). Acceptable DNA had a concentration of at least 50 ng/μl in TE (10 mM Tris/1 mM EDTA) and displayed an optical density 260/280 (OD₂₆₀/OD₂₈₀) ratio between 1.8-2.0. Whole Genome Sequencing (WGS) was performed at Illumina, Inc. (Illumina Sequencing Facility, San Diego, CA USA) or the BGI (Beijing Genome Institute, Hong Kong), as described previously (31, 32), to a target depth of 50× for the cancer samples and 30× for matched controls (31). The Burrows-Wheeler Aligner (33) (BWA) was used to align the sequencing data to the GRCh37 reference human genome.

Sequencing data generated for this study have been deposited in the European Genome-phenome Archive with the accession code EGAS00001000262. Alignment and variant calling was performed using analysis pipelines in the Cancer Genome Project (CGP) at the Wellcome Trust Sanger Institute; these can be found at https://github.com/cancerit/dockstore-cgpwgs. The Battenberg algorithm (34) was used to call clonal and subclonal copy number alterations (CNAs) in all samples https://github.com/Wedge-Oxford/battenberg. The resulting copy number profiles were subject to quality control.

A total of 123 summary measurements were generated, including some or all of the number of single nucleotide variants (SNVs), the number of indels (insertions, deletions or complex), the number of structural variants including genomic rearrangements (inversion, deletion, tandem duplication and translocation) and mutational signatures, the percentage of the genome altered (PGA), the DNA breakpoints (and whether they are involved in a chained intra- or inter-chromosomal structural variant), telomere lengths, the numbers of gene fusions, the presence or absence of any one of whole genome duplication (WGD), kataegis, ETS+ status and chromothripsis, the presence or absence of important driver mutations and copy number alterations CNA (split into loss of heterozygosity (LOH) and homozygous deletion HD)) and CNA gains.

FIG. 1b illustrates the input data which was used as training and validation data. Where applicable the top number on the y-axis corresponds to the highest value of the data (e.g. 7887 SNVs) and the dashed line denotes the median. Bar charts shows some of the measured data namely the number of SNVs, the number of indels, the number of structural variants including genomic rearrangements and mutational signatures, the PGA, the DNA breakpoints, telomere lengths and the numbers of gene fusions. There are grid plots showing the presence or absence WGD, kataegis, ETS+ status and chromothripsis. Finally, there are heatmaps showing the presence or absence of important driver mutations and CNA (split into LOH and HD) and CNA gains.

The summary measurements detailed above form the data set for further analysis. However, it contains a number of different data types (binary, categorical, ordinal, continuous), it is highly dimensional relative to the number of patients, and it undoubtedly contains highly correlated, cooccurring or equivalent events that may confound the analysis. To address this, as explained above, a feature extraction pre-processing step prior to the analysis is done. As our downstream analysis will be investigating genomic patterns that are indicative of evolutionary behaviour, it is critical that the results of these analyses can be easily interpreted. This necessitates methodology where the links between input variables that correspond to the features are identifiable.

We briefly outline how each of our summary measurements were generated, default parameters were used unless otherwise stated.

### Numbers of SNVs, Indels and Structural Variants

SNVs, insertions and deletions were detected using the Cancer Genome Project Wellcome Trust Sanger Institute pipeline as described previously (31). In brief, SNVs were detected using CaVEMan with a cut-off ‘somatic’ probability of 0.95. Insertions and deletions were called using a modified version of Pindel (35). Variant allele frequencies of all indels were corrected by local realignment of unmapped reads against the mutant sequence. Structural variants were detected using BRASS (31). Total numbers of SNVs per sample were calculated, as were total and type of indel (insertion, deletion and complex) and structural variants (large insertions or deletions, tandem duplications and translocations).

### Clonal & Subclonal SNVs

Clonal/Subclonal quantifies the number of SNVs that are in all cancer cells in the sample (clonal) or only in a subset (subclonal) i.e. SNVs with cancer cell fraction (CCF)=1 and CCF<1 respectively. These were calculated as described previously (36), by calculating the proportion of reads carrying a SNV compared to the total number of reads covering that position, followed by adjustment for tumour purity and copy number obtained through the Battenberg algorithm.

### Percentage Genome Altered

This was calculated as the percentage total of the genome that is affected by CNAs (37). We also recorded the percentage affected by clonal and subclonal CNAs (i.e. CNAs with CCF=1 and CCF<1 respectively).

### Ploidy

We adopt the same approach as detailed previously (36), where whole genome duplicated samples were those which had an average ploidy, as identified with the Battenberg algorithm, greater than 3. These samples were designated as tetraploid, otherwise the sample was diploid.

### Kataegis

Kataegis was identified using SeqKat https://github.com/cran/SeqKat.

### ETS Status

A positive ETS status was assigned if a breakpoint between ERG, ETV1, ETV3, ETV4, ETV5, ETV6, ELK4, or FLI1 and partner DNA sequences was detected and the fusion was in-frame.

### Gene Fusions

We reported the number of in-frame gene fusions, as well as those only affecting ETS genes, or only TMPRSS2/ERG.

### Breakpoints

Breakpoints were identified with Chainfinder (http://archive.broadinstitute.org/cancer/cga/chainfinder) version 1.01. Total number of breakpoints, as well as the total number of chained breakpoints (i.e. where the breakpoints are interdependent (38)), number of chains, number and proportion of breakpoints involved in the chained events, the number of breakpoints in the longest chain, and the average, median, maximum number of chromosomes involved in a chain. Information about the type of breakpoint was also recorded, including the number of deletion bridges, intra-chromosomal and inter-chromosomal events and the inter-chromosomal to intra-chromosomal ratio.

### Mutated Driver Genes.

A set of driver genes were identified from our previous publication (36). Using the CaVEMan output, we determined any non-synonymous mutations in the exonic regions of these genes as a positive event in our data set.

### Copy Number Alterations.

We followed our previous approach (36) to identify consistently aberrant regions. A permutation test was developed where CNAs detected from each sample were placed randomly across the genome and then the total number of times a region was hit by each type of CNA in this random assignment was compared to the number of times a region was hit in the actual data. This process was repeated 100,000 times and recurrent (or enriched) regions were defined as having a false discovery rate (FDR) of less than 0.05. This was performed separately for gains, loss of heterozygosity (LOH) and homozygous deletions (HD). We identified small regions initially and these were amalgamated into larger regions defined as the amalgamation of adjacent regions all of which had an FDR less than 0.05. For each sample, if a breakpoint corresponding to a gain, LOH or HD occurred in each region, then the respective datum was set to 1, and 0 otherwise.

### Telomere Lengths.

Telomere lengths were estimated as described in our previous publication (39). A mean correction was applied to batches to compensate for the effects of a change in chemistry during the project.

### Chromothripsis.

The identified copy number breakpoints were segmented according to the inter-breakpoint distance along the genome using piecewise constant fitting (pcf from the R package copynumber v1.22.0). Regions with a density higher than 1 breakpoint per 3 Mb were flagged as high-density regions. A chromothripsis region was then defined as a high-density region with a number of copy number breakpoints N>15; a non-random segment size distribution (Kolmogorov-Smirnov test against the exponential distribution, P<0.05); at most three allele-specific copy number states covering more than min (1, 0.006N+1.1) fraction of the region; and the proportion of each type of structural variant is random with equal probability PTD=PDel=PH2Hi=PT2Ti=0.25 (multinomial test P>0.01), where TD=tandem duplication, Del=deletion, H2Hi=head-to-head inversion and T2Ti=tail-to-tail inversion.

### Clustering Classification

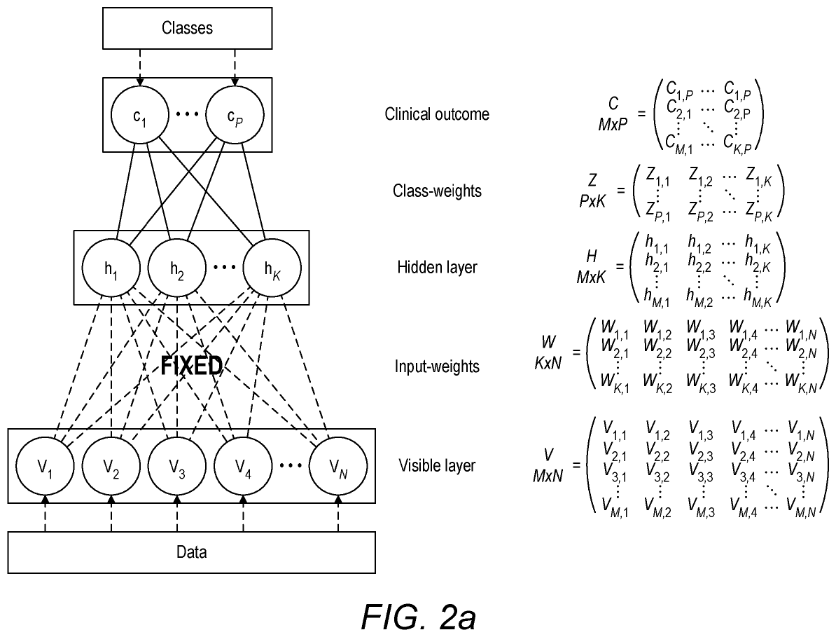

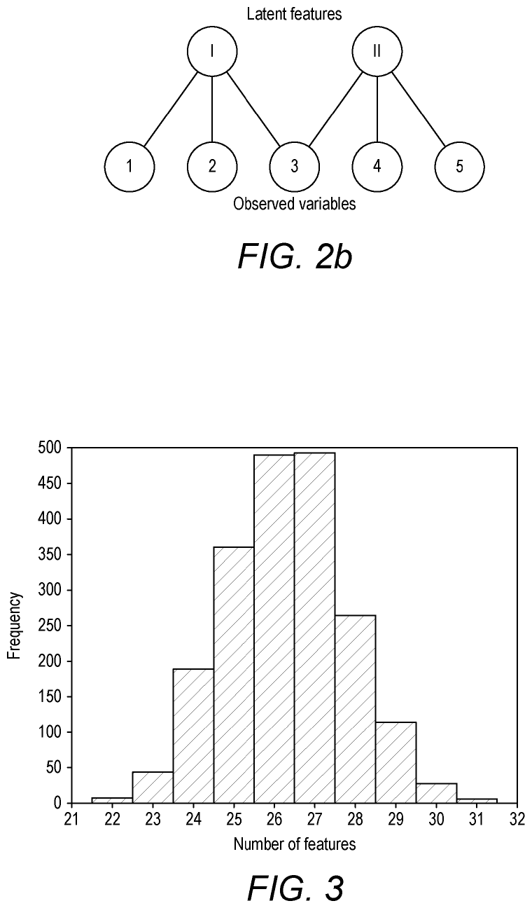

FIG. 2a illustrates a model which has been trained as explained below to generate the reduced set of features and to score the features relative to relapse occurrence. In the example of FIG. 2a, the model is a modified Restricted Boltzmann Machine (44) (RBM) neural network. Latent feature (or latent variable) analysis provides a way of reformulating input data into a reduced set of features that encapsulate the underlying relationships between the original inputs. This framework can be described using graphical models, such as in FIG. 2b where two latent features contribute to five observed variables. Note that the lack of a connection between the latent features indicates they are conditionally independent. Downstream analysis can then be performed on the latent features directly.

There have been many latent feature models proposed, each with associated inference methods for the features (a process called feature learning). These included methods such as non-negative matrix factorisation (41), Bayesian non-parametric methods (42) and neural networks (43). However, none of these known models was able to fulfil all of our requirements and we therefore created a bespoke RBM neural network.

An RBM is extensible to multiple data types (45, 46) and can provide interpretable hidden units, with appropriate modifications (47). A basic RBM unit consists of only two layers, known as the visible and the hidden layers and one weight matrix, which is used to update both the visible and hidden layers. Both of these layers and the weight matrix are present in the bespoke RBM as shown in FIG. 2a. The information on the transformation from visible units (input representation) to the hidden units (feature representation) is encapsulated in the weight matrix. Hence, we also refer to it as the input-feature map. In FIG. 2a, the weight matrix is described as fixed but as explained in detail below, the fixing of the weight matrix occurs partway through the training process. Thus, FIG. 2a depicts the trained state of the RBM.

The bespoke RBM of FIG. 2a is adapted to calculate the discrimination scores for each feature as described in more detail below. The RBM thus includes an extra classification layer, which is fully connected to the hidden layer. There is another set of weights that denote the strength of the connection between the hidden and classification layers.

The hidden layer typically has fewer units than the visible layer. The basic RBM is formulated as a probabilistic network, meaning each unit represents a random variable rather than a fixed value. All the units can take only values of 1 or 0 (active or inactive respectively), and the inputs to each unit represent the probability that the unit is active. The visible layer therefore represents a distribution over the observed data values, and the hidden layer represents a distribution of the hidden units. The RBM needs to be trained as described below and it is noted that training occurs in a step-wise fashion, in which each layer is sampled in turn and used to update the weights and parameters of the other layer (the input data is used in the initial hidden unit samples). Biases which adjust the baseline activation probability of each unit are not shown for ease of understanding.

Merely as background, it is noted that an RBM is functionally similar to another type of neural network architecture called an autoencoder (43). The hidden layer of the RBM performs a similar function to the code layer in the autoencoder, albeit with a probabilistic representation. It has also been shown that the RBM is equivalent to the graphical model of factor analysis (48) and so each hidden unit can be interpreted as a latent feature.

The standard RBM formulation (44) has Bernoulli random variables for all visible v={vᵢ} and hidden units h={hᵢ}, where vᵢ, hⱼ∈{0,1}, with respective biases a={aᵢ}, b={bⱼ}; aᵢ, bⱼ∈(−∞, ∞), and a matrix of weights, W; wᵢⱼ∈(−∞, ∞). Training of an RBM is based on minimising the free-energy of the visible units, as a low free-energy corresponds to a state where the data is explained well through the model parameterisation. Energy-based probability distributions take the form

$$P ( v , h ) = \frac{{e}^{- E ( v , h )}}{Z} , \qquad ( 1 )$$

where E(v,h) is the energy function and Z is a normalising factor. This is the probability of observing the joint v, h pair. The energy function in an RBM is given as

$$𝔼 ( v , h ) = - {a}^{T} v - {b}^{T} h - {v}^{T} W h \qquad ( 2 )$$

In this formulation,

$$Z = {\sum }_{v} {\sum }_{h} {e}^{- E ( v , h )} , \qquad ( 3 )$$

which is difficult to calculate due to the number of possible combinations of v and h.

Training is conducted with respect to the energy at the visible units and, thus, we need to marginalise over h in Equation 1 to calculate the likelihood of observing the visible unit corresponding to a single data sample dₖ from data set D={dₖ, k=1, 2, . . . , K}. The likelihood is calculated using:

$$ℒ ( θ \vert v = {d}_{k} ) = \frac{1}{Z} {\sum }_{h} {e}^{- E ( {d}_{k} , h )} \qquad ( 4 )$$

where θ∈{{aᵢ}, {bⱼ}, {wᵢⱼ}} is the full parameter set. To simplify notation, we write [symbol] (θ|v=dₖ) as [symbol] (dₖ) with no loss of generality. To perform training through gradient descent, we need to calculate the gradient of the negative log-likelihood for each parameter we wish to update, ∂(−log [symbol] (dₖ))/∂θ. The partial derivative of the logarithm of Equation 4 takes the form

$$\frac{\partial }{\partial θ} ( - log ℒ ( {d}_{k} ) ) = \frac{\partial }{\partial θ} ( log {\sum }_{h} {e}^{- E ( {d}_{k} , h )} ) - \frac{\partial }{\partial θ} ( log {\sum }_{v} {\sum }_{h} {e}^{- E ( v , h )} ) \qquad ( 5 )$$

$$= {\sum }_{h} P ( h \vert v = {d}_{k} ) \frac{\partial E ( {d}_{k} , h )}{\partial θ} - {\sum }_{v} {\sum }_{h} P ( v , h ) \frac{\partial E ( v , h )}{\partial θ} . \qquad ( 6 )$$

We then calculate the expected values using the entire training set

$${E}_{D} [ \frac{\partial }{\partial θ} ( - log ℒ ( {d}_{k} ) ) ] = {𝔼}_{P ( h \vert D )} [ \frac{\partial E ( v , h )}{\partial θ} ] - {𝔼}_{P ( v , h )} [ \frac{\partial E ( v , h )}{\partial θ} ] , \qquad ( 7 )$$

which can be used to update the model parameters via gradient descent. The [symbol] _{P(h|D)} term corresponds to the expected energy state invoked from observing the data samples, and the [symbol] _{P(v,h)} is the expected energy state of the model configurations, both contingent on the current model parameters. As such, they are often called [symbol] _{data} and [symbol] _{model} respectively. Calculating the partial derivatives with respect to the parameters gives

$$\frac{\partial }{\partial {w}_{ij}} ( - log ℒ ( {d}_{k} ) ) = 𝔼 [ {v}_{i} {h}_{j} \vert v = {d}_{k} ] - 𝔼 [ {v}_{i} {h}_{j} ] , \qquad ( 8 )$$

$$\frac{\partial }{\partial {a}_{i}} ( - log ℒ ( {d}_{k} ) ) = 𝔼 [ {v}_{i} \vert v = {d}_{k} ] - 𝔼 [ {v}_{i} ] , \qquad ( 9 )$$

$$\frac{\partial }{\partial {b}_{j}} ( - log ℒ ( {d}_{k} ) ) = 𝔼 [ {h}_{j} \vert v = {d}_{k} ] - 𝔼 [ {h}_{j} ] , \qquad ( 10 )$$

which are used to construct the update equations

$${W}^{n e w} ← {W}^{o l d} + v ( {𝔼}_{d a t a} [ {v}^{T} h ] - {𝔼}_{m o d e l} [ {v}^{T} h ] ) , \qquad ( 11 )$$

$${a}^{n e w} ← {a}^{o l d} + η ( {𝔼}_{d a t a} [ v ] - {𝔼}_{m o d e l} [ v ] ) , \qquad ( 12 )$$

$${b}^{n e w} ← {b}^{o l d} + η ( {𝔼}_{d a t a} [ h ] - {𝔼}_{m o d e l} [ h ] ) , \qquad ( 13 )$$

or learning rates ν and η. The [symbol] _{data} values can be estimated easily by taking the arithmetic mean.

The [symbol] _{model} terms are generally difficult to calculate as they involve summation over all possible configurations of v and h. An alternative is to perform Gibbs sampling using the conditional probabilities as these are far easier to calculate due to the conditional independence between units in the same layer. We can estimate the conditional probability of values of the hidden layer from the visible layer and vice versa thus

$$( h \vert v ) = {\prod }_{j} P ( {h}_{j} \vert v ) , \qquad ( 14 )$$

$$P ( v \vert h ) = {\prod }_{i} P ( {v}_{i} \vert h ) . \qquad ( 15 )$$

The form of P(hⱼ|v) and P(vᵢ|h) depends on the activation function. This function that inputs the products of the units in one layer and their corresponding weights, and outputs a probability that a unit is active. In this study, we use a logistic sigmoid (or simply “sigmoid”) function, which is given by

$$σ ( x ) = \frac{1}{1 + {e}^{- x}} , \qquad ( 16 )$$

where x is dependent on the layer we are sampling, and so the individual hidden and visible probabilities can be written as

$$P ( {h}_{j} \vert v ) = σ ( {b}_{j} + {\sum }_{i} {v}_{i} {w}_{i j} ) , \qquad ( 17 )$$

$$P ( {v}_{i} \vert h ) = σ ( {a}_{i} + {\sum }_{j} {h}_{j} {w}_{i j} ) . \qquad ( 18 )$$

A sample is drawn by setting the corresponding unit to 1 with probability given by the value for P(hⱼ|v) or P(vᵢ|h) as appropriate. These can then be used to calculate estimates for P(v) and P(h) by marginalisation over the conditional variable. In practice a full Gibbs sample every update iteration would be prohibitively slow and so we used an approximation called contrastive divergence (44), in which the Gibbs sampler is initialised using the input data and a limited number of Gibbs steps are performed. In our implementation we use one contrastive divergence step (i.e. CD (1)), and so the data (or mini-batches of the data) are presented as a matrix and used to sample the hidden unit values, which are then used to update the values of the visible units. These values are used to update the network parameters using stochastic gradient descent (SGD) (49). As such, the information travels both ways across the weights during these initial stages.

During training, the results of these updates are stored in three matrices (H, W and V) that correspond to the weights as well as the network representation of the tumour data at the visible and hidden layers. These matrices correspond to the network reconstruction of the data (visible layer, V) the latent feature representation of the data (hidden layer, H), and the input-feature mapping (weights, W). When the network is trained, these can be extracted and utilised in the analysis.

A number of simple modifications were made to a standard RBM to ensure the feature representation was interpretable, generalisable, stable and reproducible. These modifications include data integration, use of non-negative weights, hidden unit pruning, sparsity, avoidance of overfitting and convergence to a global solution. It will be appreciated that although all of the modifications are incorporated as described below, alternative versions could incorporate some but not all of the modifications.

### Data Integration

Our data consisted of multiple different modalities; unlike conventional multiomic approaches which have a large number of a data points from a small number of sources, we have a small number of data points from a large number of sources. As such, data integration needed to be carefully considered. The RBM can be modified to incorporate inputs of multiple modalities, sometimes through modification of the energy function (50,51). However, we decided to avoid this complication and standardise all our inputs by ranking all integer and continuous variables prior to rescaling to [0, 1]. As an example, the specific transformations which were incorporated are:
Binary—set as {0, 1},
Categorical—one-hot encoding*
Integer—rank and scale to [0, 1],
Continuous—rank and scale to [0, 1].

For the integer and continuous cases, we used ranking as this decouples the value from the distribution of the inputs and after scaling to [0, 1], the new value can be interpreted as the probability that the corresponding visible unit is active. As such, all inputs are treated equally in the machinations of the RBM. These transformations do not affect the hidden units, which remain a Bernoulli random variable, hᵢ∈{0,1}. In one-hot encoding, the categorical variable is replaced with a vector of the same length as the number of categories. The values of the vector are all zero except for a one at the n^{th} position which indicates membership of the m^{th} category.

### Non-Negative Weights

Neural networks are considered as black-box approaches because the transformations they perform are highly complex. To improve interpretability of the network machinations we imposed a non-negativity constraint to the weight updates, specifically by penalising negative values. We use an approach in which a quadratic barrier function is subtracted from the likelihood for each negative weight (47). Mathematically, this is written as

$${ℒ ( {d}_{k} )}_{n o n n e ρ} = ℒ ( {d}_{k} ) - \frac{α}{2} {\sum }_{i} {\sum }_{j} f ( {w}_{i j} ) , \qquad ( 19 )$$

where α denotes the strength of the penalty, and

$$f ( x ) = \{ \begin{matrix}{x}^{2} , & ifik < 0 , \\ 0 , & otherwise\end{matrix} . \qquad ( 20 )$$

This leads to the update rule

$${W}^{n e w} ← {W}^{o l d} + v ( {𝔼}_{d a t a} [ {v}^{T} h ] - {𝔼}_{m o d e l} [ {v}^{T} h ] - α {W}^{\{ - \}} ) , \qquad ( 21 )$$

W^{{-}} is a matrix containing the negative entries of W, with zeros elsewhere. This formulation is equivalent to a L₂-norm penalty on the negative weights, and so penalises more strongly negative weights to a greater degree. When used in the training scheme, this coerces network weights to non-negative solutions, simplifying the interpretation of the input-feature map. This can be considered to be a non-linear extension of non-negative matrix factorisation (41), and similarly can be used to represent the underlying structure of the data by its parts, which are the features in machine learning terminology.

As weights can no longer trade off against each other with counteracting weights of opposing signs, this means that the lowest free-energy state corresponds to a state with minimal redundancy and so during training the hidden units compete to convey information about a single input (52). This means that the input will only be represented in a small number of latent variables, so when the initial number of hidden units is of similar order to the number of data inputs, this results in some of the biases or weights converging to a negligible value, and the corresponding hidden layer activations converge to an arbitrary fixed value. The latter are then called dead units. This is of fundamental importance to our method as it can be used as an estimate of the intrinsic dimensionality of the data.

### Hidden Unit Pruning

During training, we prune the dead units to improve the speed of the algorithm. However, determining dead units is not straightforward in a probabilistic network such as the RBM as the values in the network at each state will vary stochastically. To circumvent this, we apply an L_{1/2}-norm penalty on the hidden unit activations, which penalise a non-zero activation value (53). This coerces the values for all patient samples to be zero, rather than some arbitrary value, and these can then be easily identified and removed with a thresholding approach. This penalty function is calculated over all training data samples, so for consistency with Equation 4 we can formulate the likelihood for each sample as

$${L ( {d}_{k} )}_{activ} = L ( {d}_{k} ) - \frac{β}{K} {\sum }_{k} {‖ f ( {y}_{k} ) ‖}_{1 / 2} , \qquad ( 22 )$$

where ƒ(yₖ)=P(h|vₖ) and β is a parameter describing the strength of this penalty. We calculate the gradient of the additional likelihood term with respect to each of the hidden unit biases, which is given as

$$Δ {b}_{j}^{( {L}_{1 / 2} )} = \frac{1}{K} \frac{\partial {Σ}_{k} {‖ P ( h \vert {v}_{k} ) ‖}_{1 / 2}}{\partial {b}_{j}} , \qquad ( 23 )$$

$$= \frac{1}{2} {\sum }_{k} \frac{exp ( - {b}_{j} - {\sum }_{i} {v}_{ik} {w}_{ij} )}{{\vert 1 + exp ( - {b}_{j} - {\sum }_{i} {v}_{ik} {w}_{ij} ) \vert}^{3 / 2}} . \qquad ( 24 )$$

We can then write the vector of gradients for all hidden unit biases as Δb^{(L}^{_{1/2}}⁾. The corresponding update rule can therefore be written as

$${b}^{new} ← {b}^{old} + η ( {𝔼}_{data} [ h ] - {𝔼}_{model} [ h ] ) - β Δ {b}^{( {L}_{1 / 2} )} \qquad ( 25 )$$

In our training algorithm, we prune dead units every 50 iterations after the first 1000 iterations.

### Sparsity

Sparsity is a desirable property for latent space representations, as it means that the information is conveyed in a concise form. The penalty measure defined in Equation 22 introduces sparsity as it penalises hidden units which are highly active thus coercing the network toward a sparse configuration (53). Further sparsity measures were not used in training as the weight matrix, which defines the input to feature mapping, will be stringently filtered at a later stage.

### Avoidance of Overfitting

A concern with any neural network formulation is the tendency to overfit the data, which in this application would lead to a feature set that was not representative of the true underlying structure, and therefore not generalisable. To mitigate this, we employed a number of countermeasures, for example:
1. DropConnect,
2. Max-norm regularisation,
3. Bootstrap aggregating,
4. Early Stopping.

With DropConnect (54), a predetermined proportion of weights in the network are randomly set to zero with uniform probability at each training iteration. This helps prevent overfitting by temporarily disrupting correlations between features, so they are more likely to learn features that are independent of the state of other features.

When using max-norm regularisation (55), we set an absolute value on the norm of each weight vector that form the input to a single hidden unit. If a vector becomes too large, then we rescale the vector so that it obeys the constraint. It is possible for non-negative weights to continue increasing throughout training as the binary nature of some inputs means that when present they were already in the maximal output of the sigmoid activation function, so the precise value is irrelevant. Max-norm regularisation prevents this occurrence and facilitates comparison between weight matrices of different runs.

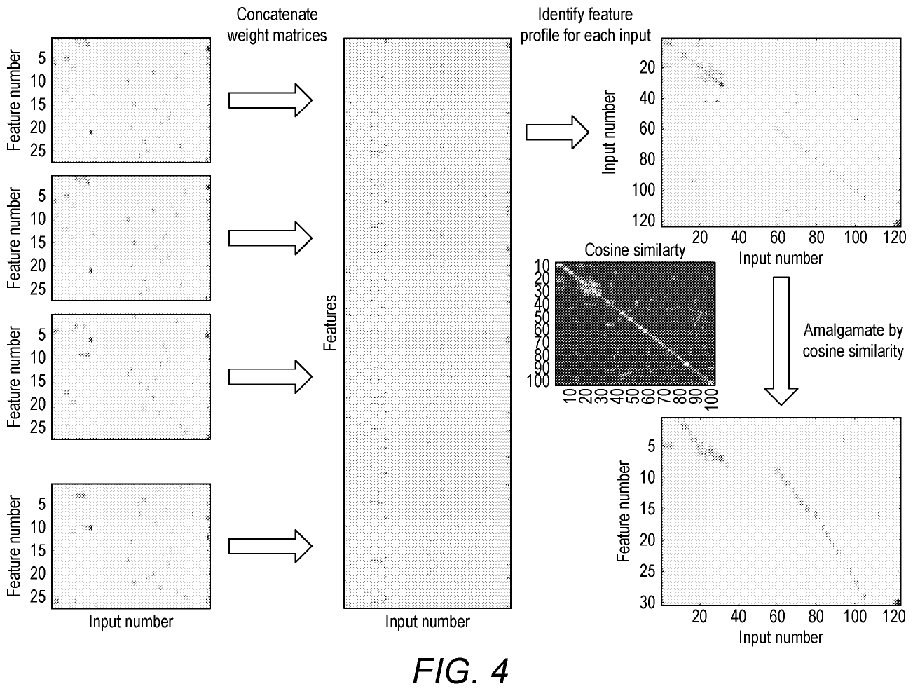

For bootstrap aggregating (56) (bagging), multiple networks with the same initial architecture were trained on subsets of the data and the outputs amalgamated. In our feature learning representation, we extracted the weight matrix from each of the networks and merged them according to the cosine distance between features as shown in FIG. 4 and explained in more detail below.

Finally, when implementing early stopping (57) we need to compare the performance of the network on the training set to the performance on an unseen validation set. If the network performs similarly on the training and validation sets then it is a good indicator that it will return generalisable outputs. Beginning with the subsets extracted for ensemble learning, we use data omitted when the subset was sampled as the validation set, which is propagated through the network. As the RBM is formulated as an energy-based model, early stopping is predicated by comparing the free energy in the training set to the free energy of the validation set (58). If the free energy arising from the training set becomes consistently lower than that of the validation set, then overfitting is occurring, and training is stopped.

### Convergence to Global Solution

As we are training multiple networks and amalgamating the results, it is important that each network converges to the global solution or the results will be incongruous. Furthermore, as the RBM is trained by stochastic gradient descent, it is possible that the algorithm may get stuck in a local optimum. To minimise the chance of this occurrence, we used the cyclical learning rate scheme (59), in which learning rates for each of the variables oscillates between zero and a maximal value throughout training. The maximal value is subject to decay so that the maximal training rate will diminish throughout training to zero. This approach has been shown to help convergence to the global solution and has the advantage that the learning rate parameters do not need to be tuned (59).

We trained 2000 networks using 75% of the data as the training set (chosen uniformly at random). The remainder of the data was used as a validation set for early stopping. If early stopping occurred then the entire network was discarded (as it may not have had time to converge to an accurate feature representation) and another trained in its place, this was repeated until training finished normally. FIG. 3 illustrates that the dimensionality of the extracted features trained as above is consistent with a mean of 26.30 and a standard deviation of 1.51.

As explained above, a plurality of networks are trained with the data and each individual network run provides a similar, but not identical, weight matrix. As such, weight matrices from each network run were amalgamated and filtered to form the final input-feature map. Numbers of features, the inputs they represent, their magnitude and order would not necessarily occur the same in each network and so we constructed an algorithm based on the cosine similarity.

FIG. 4 schematically illustrates the steps of the algorithm. Each heatmap of FIG. 4 shows the relative magnitude of network weights corresponding to the map between each input and each feature. The individual weight matrices on the left of the Figure are concatenated to form a large matrix in the middle of the Figure. Co-occurring inputs and their relative magnitudes are calculated for each input to form the preliminary feature set. Cosine distance is then calculated pairwise between the new features and used to amalgamate features that were within a similar threshold.

An example of the pseudocode for amalgamating weight matrices is shown in the algorithm below:

Algorithm 1:

```
input : set of weight matrices from each network run
concatenate weight matrices into matrix W;
set low magnitude weights to zero;
set similarity threshold τ = 0.5;
Initialise matrix M with number of rows and columns equal to number of inputs;
Initialise empty feature matrix F;
for i=1 to number of inputs do
  set i^{th} row of M equal to mean of all rows of W where the i^{th} weight > 0
end
calculate pairwise cosine similarity matrix | from M;
while number of rows in S>0 do
  read in the first row of S as the current similarity vector s;
  identify all / where sⱼ > τ ;
  add the mean of all M[j,:] as a row to F;
  remove j^{th}
  rows from S and M;
end
rescale all rows in feature matrix F by max-norm;
```

Low magnitude weights were those less than 50% of the maximum weight value for each hidden unit. The amalgamated weight matrix has 30 features, as opposed to 22-31 in each individual run. This is mainly due to low frequency inputs not being consistently represented after the data is subsetted for cross-validation.

Returning to FIG. 2a, the amalgamated weight matrix is used as the fixed weight matrix. The feature representation of the data is then obtained using the network in FIG. 2a but utilising all patient samples and the fixed weight matrix. This was done by initialising the weights to the amalgamated weight matrix and setting the weight learning rate to zero. Learning of the biases was enabled, as these may be different to the biases in the previous networks due to the removal of low magnitude weights.

Once the remaining network parameters have converged during training, taking further iterations is equivalent to sampling the hidden units/feature representation for each patient. We therefore averaged the hidden unit values taken every 10 iterations during the final 1000 iterations to obtain the final feature representation.

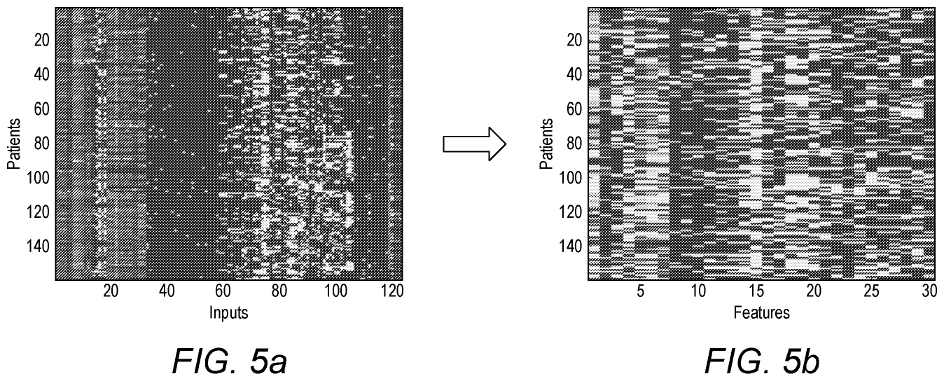

FIG. 5a illustrates the heatmap showing the relationship between the patients and the input data. FIG. 5b illustrates how the data is transformed to a feature representation as described above. FIG. 5b shows that the 123 inputs have been reduced to the 30 features set out in the table which shows the reduced set of features and which is described in relation to FIG. 1a.

### Two-Stage Clustering

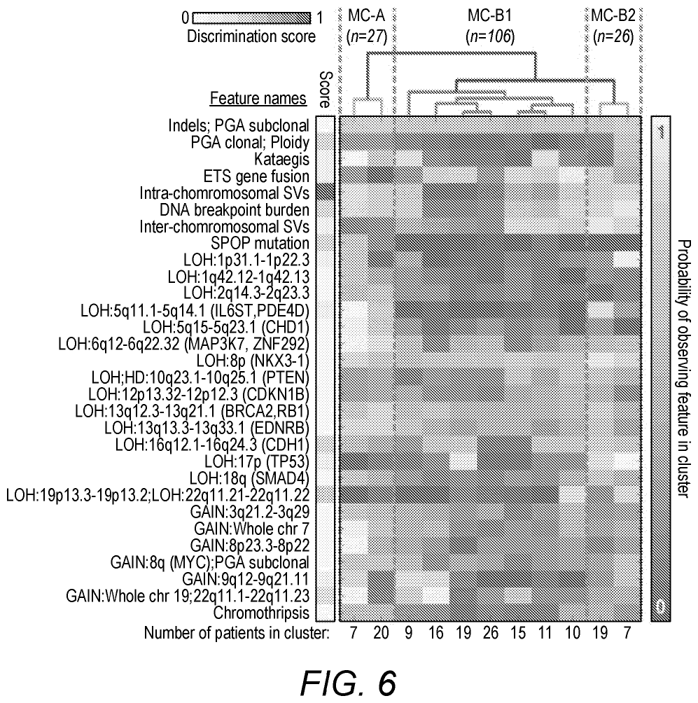

The dimensionality of the feature representation of FIG. 5b is still quite large for conventional clustering techniques. Therefore we adopted a two-stage approach where we first clustered by those features that were most informative of clinical outcome, calculated the centroids of these first-stage clusters for all features, and then clustered these in the second-stage of clustering to produce the results shown in FIG. 6. More details on identification of informative features using a discrimination score and the clustering methods used are set out below.

### Discrimination Score

There have been several methods proposed for quantifying the relative importance of the units of a neural network (60). However, most of these are generally formulated to discover the inputs that are important in discerning the output (61, 62). In our application, we wish to quantify the discriminative capacity of each of the features (hidden layer) with respect to the clinical outcome. As we utilise non-negative weights to determine the relevance of the inputs to the hidden units in the feature extraction, we can adopt a similar approach to determine the importance of the hidden units to the outcome.

As described above briefly with reference to FIG. 2a, the architecture of a base RBM is modified so that it was similar to ClassRBM (63) and so that the discrimination scores for each feature can be obtained. Thus, as shown in FIG. 2a, the RBM of the present techniques comprises an extra classification layer, which is fully connected to the hidden layer, the units of which contain the values of the classes. We wish to uncover underlying relationships in the data (encapsulated by the features) in an unbiased way, and then determine how relevant these features are to the clinical outcome. We therefore enforced that the classification weights were uni-directional, and information used in training was only passed from the hidden layer to the classification weights. This ensures that the latent structure encapsulated by the hidden units remains unbiased by the knowledge of the clinical outcome, and the algorithm for feature learning can still be considered as unsupervised. (By contrast in ClassRBM, there is another set of weights that denote the strength of the connection between the hidden and classification layers, and these are trained in the same bi-directional fashion as the input weights.)

Furthermore, we enforced a non-negative constraint on these class-weights, similar to the input-weights. As such, when trained, the relative magnitude of these class-weights quantifies how important each corresponding feature is in distinguishing the corresponding clinical outcome, in a similar fashion to standard non-negative matrix factorisation. We take the absolute value of the weights corresponding to relapse minus the weights corresponding to no-relapse to get our discrimination score, s. This can be expressed mathematically as

$$s = \vert {c}_{r} - {c}_{{r}^{′}} \vert \qquad ( 26 )$$

where cᵣ are the class-weights associated with relapse, and cᵣ, are those associated with no relapse.

These s values can be considered as heuristic and quantify the importance of the corresponding feature to the clinical output, similar to how the component loadings quantify the explained variance of the corresponding principal component in principal component analysis (PCA). There is no set rule for determining the number of features, so we followed a similar approach to that conventionally used in PCA and selected the number of features using the cumulative distribution. We chose a cut off of 0.9 of the total cumulative discrimination score, which resulted in 14 out of 30 features being selected for the initial clustering phase. These 14 features are listed below and shown highlighted (in red) in FIG. 6:

Table of features for initial clustering phase

Feature (or chromosome region)
PGA clonal; ploidy
Kataegis
ETS gene fusion
Intra-chromosomal SVs
DNA breakpoint burden
Inter-chromosomal SVs
SPOP mutation
LOH in 1p31.1-1p22.3
LOH in 5q22.1-5q14.1 (IL6ST, PDE4D)
LOH in 16q12.1-16q24.3 (CDH1)
LOH in 17p (TP53)
LOH in 19p13.3-19p13.2; LOH in 22q11.21-22q11.22
Gain in 9q12.9-9q21.11
Gain in whole chr 19; 22q11.1-22q11.23

### Clustering

Clustering of tumours was performed on the latent feature representation in a two-stage process to facilitate the identification of clusters that were relevant to clinical outcome. As the feature representation for each patient can be considered as a vector containing the probabilities that the corresponding feature is active, it is appropriate to use a distance measure that quantifies the distance between probabilities. As such, we calculated the mean Jensen-Shannon (J-S) divergence (64) between tumours in a pairwise fashion.

For a pair of patients, A and B, represented by the latent feature representation in hidden layers h_{A} and h_{B}, the mean J-S divergence can be written as

$$JSD ( {h}_{A} \vert \vert {h}_{B} ) = \frac{1}{2 K} {\sum }_{i = 1}^{K} [ {h}_{A , i} log ( \frac{{h}_{A , i}}{{m}_{i}} ) + {h}_{B , i} log ( \frac{{h}_{B , i}}{{m}_{i}} ) ] , \qquad ( 27 )$$

where where

$$m = \frac{1}{2} ( {h}_{A} + {h}_{B} ) ,$$

is the midpoint of h_{A} and h_{B}. The additive terms in the square brackets in Equation 27 represent the Kullback-Leibler divergence between each element of the latent feature representation for either patient and the corresponding element of the midpoint vector, m.

As we are not using a Euclidean distance metric, clustering through k-means is not appropriate and so we used k-medoid clustering for the first stage; this is similar to k-means but selects a representative data point (medoid) as the centroid for each cluster instead of the mean. Using the silhouette method (65), we determined that 11 clusters was optimal. For the second stage of clustering, we used hierarchical clustering to cluster the medoids themselves (again using the J-S divergence), and this was used to generate and order clusters by the dendrogram shown in FIG. 6.

FIG. 6 shows the discrimination score quantifying the relevance of each feature in predicting relapse as a green heatmap. Fourteen features (red and listed in the table of features for the initial clustering phase) are used as inputs for the k-medoid clustering with 11 clusters (determined by the silhouette method).

The medoids of each cluster were used as inputs to hierarchical clustering using all features, which revealed two main metaclusters, MC-A and MCB, with different profiles. Metacluster MC-B was further separated into MC-B1 and MC-B2 as indicated by the dendrogram. The main heatmap shows the medoid feature values for the patients in each cluster, ordered by hierarchical clustering (scale on the right). Metacluster colours are denoted by text above the dendrogram.

Thus, Metacluster A (MC-A) may be identified by a sample having intra-chromosomal structural variants, SPOP mutations, chromothripsis and loss of heterozygosity (LOH) in regions 5q15-5q23.1 (spanning CHD1) and 6q14.1-6q22.32 (MAP3K7, ZNF292). Metacluster B1 (MC-B1) may be identified by a sample having ETS fusions and loss of heterozygosity (LOH) affecting 17p (TP53) and regions 19p13.3-19p13.2; 22q11.21-22q11.22. Metacluster B2 (MC-B2) may be identified by a sample having frequent ETS fusions, inter-chromosomal chained structural variants and loss of heterozygosity (LOH) affecting 17p (TP53) and regions 5q11.1-5q14.1 (IL6ST, PDE4D) and 10q23.1-10q25.1 (PTEN).

### ARBS Classification (Classification by DNA Breakpoint Proximity to Androgen Receptor Binding Site)

To examine the proximity of DNA breakpoints to androgen receptor binding sites (ARBS), we designed a permutation approach that quantifies the departure from a random distribution of the breakpoints across the genome. We downloaded the processed ChIP-seq data targeting AR for 13 primary prostate cancer tumours from Gene Expression Omnibus (accession GSE70079) (66) and amalgamated them for use as the ARBS locations.

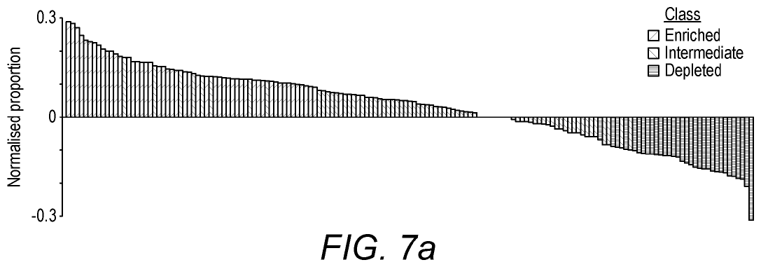

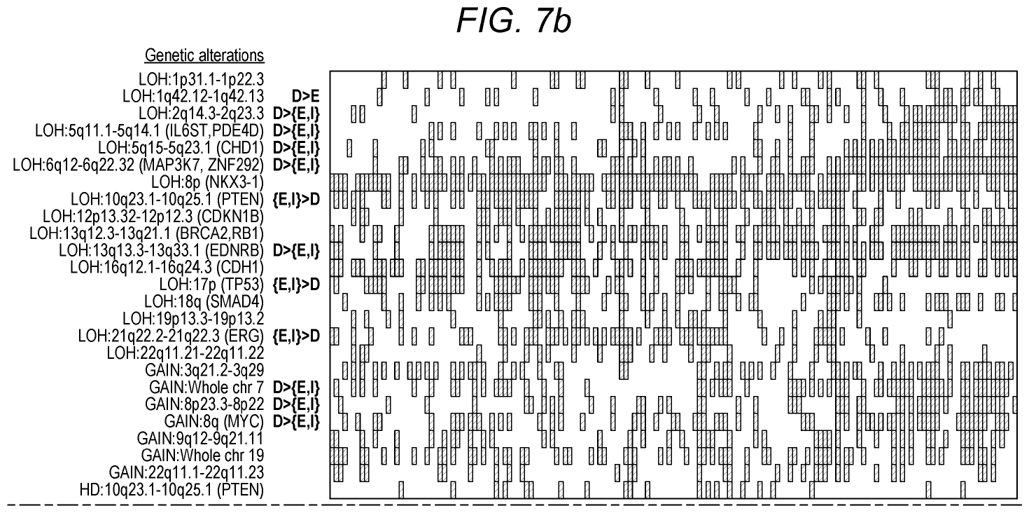

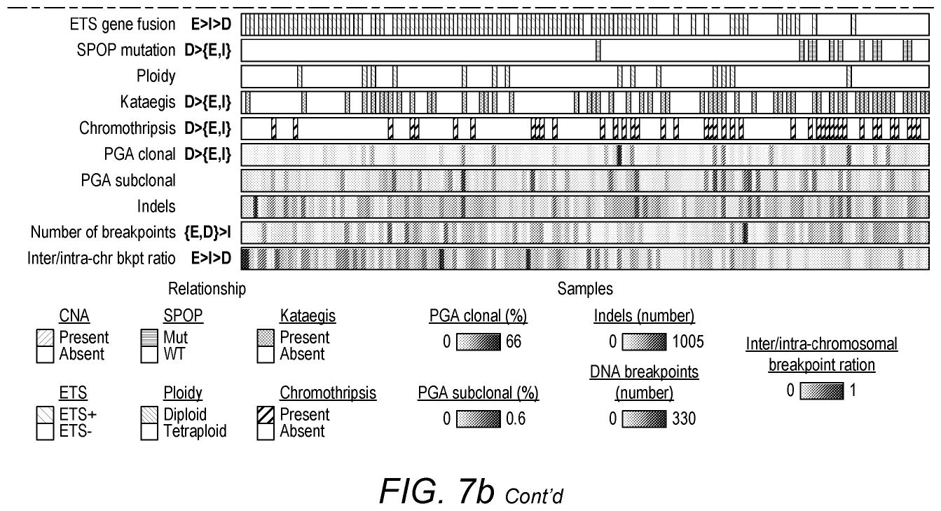

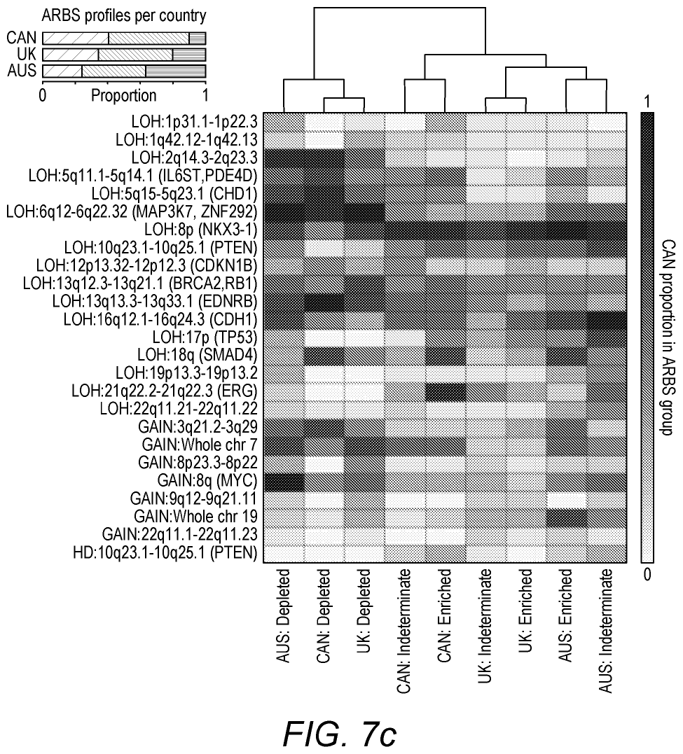

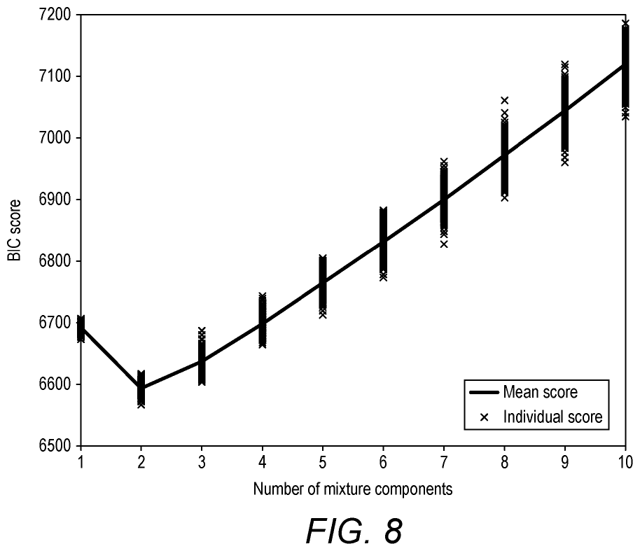

To detect significant departure from a uniform random distribution, we calculated the proportion of breakpoints within 20,000 base pairs (bp) of an ARBS for the observed and permuted data (B_{obs} and Bₚₑᵣₘ, respectively). If B_{obs}>P_{97.5%}(Bₚₑᵣₘ), the tumour was classified as Enriched, else if B_{obs}<P_{2.5%}(Bₚₑᵣₘ), the tumour was classified as Depleted. Otherwise the difference is not significant and the tumour was classified as Undefined. The level of enrichment or depletion of breakpoints in the proximity of ARBS used in FIG. 7a was estimated according

$$D = {B}_{obs} - {\tilde{B}}_{perm} \qquad ( 28 )$$

The method was validated using the same data used to train the modified RBM above. FIG. 7a shows the results of calculating the proportion of DNA breakpoints within 20 kilobases (kb) of an AR binding site for each patient in the 159 samples. For each of our 159 samples, we randomly shuffled the observed breakpoints across the genome (GRCh37) masked for assembly gaps (AGAPS mask) and intra-contig ambiguities (AMB mask) 1000 times using the R package RegioneR (67). In FIG. 7a, the number of breakpoints is normalised by the number of proximal breakpoints expected by chance. Each of the tumour samples are ordered according to this normalised proportion. Classes (enriched, depleted or indeterminate) were determined based on whether the tumour displayed more proximal breakpoints than expected (enriched), fewer proximal breakpoints than expected (depleted) or no statistically significant difference (indeterminate).

FIG. 7b shows heatmaps of genomic features for each patient using the ordering from FIG. 7a. The genomic features include the genetic alterations associated with the previously identified features from the modified RBM. As shown, Depleted tumours had the highest percentage genome altered (PGA) and the highest frequency of multiple CNAs, chromothripsis, kataegis, and SPOP mutations (Relationship column, FIG. 7b). Enriched and indeterminate tumours displayed no significant differences for any CNAs, but both showed higher frequency of CNAs covering PTEN and TP53 than the Depleted group (Relationship column, FIG. 7b). In the case of ETS fusions and inter/intra-chromosomal cSV ratio, the Enriched group showed greater enrichment than the intermediate group, which in turn showed greater enrichment than the Depleted group. Both Enriched and Depleted tumours displayed higher numbers of breakpoints than Indeterminate tumours. The associations with ARBS pairs were established with a one-tailed Mann-Whitney U-test with P<0.05.

In FIG. 7b, statistically significant relationships for the three classes are shown in the “relationship” column, where E, D and I indicate enriched, depleted or indeterminate respectively. Braces {.,.} indicate no relationship between the enclosed classes, but they both display significant differences to the remaining class. Relationships are ordered so the leftmost class(es) are those showing significantly greater proportion of genetic alteration. For Bernoulli variables, significance was determined with the Chi-squared test followed by a Fisher exact test for each pairwise relationship, for continuous variables a Kruskal-Wallace test with Tukey's HSD was used (adjusted P<0.005 for all tests).

FIG. 7c shows the ARBS groups in two additional data sets compared to the 159 samples from the ICGC UK (UK) data set. The purpose of this analysis was to validate the ARBS findings in additional datasets. The first set is a set of low-intermediate risk tumours from the Canadian Prostate Cancer Genome Network (CPC-GENE) (12) and the second set is a set of high-risk tumours from the Melbourne Prostate Cancer Research Group in Australia (unpublished). The bar plot in the top left shows the proportion of each ABRS group in each country's data. The main figure shows the results of clustering these groups by CNA proportions. We found that the depleted groups clustered together across all data set (P<0.0337; Approximate Unbiased Multiscale Bootstrap).

ARBS clusters were identified with a bespoke permutation test with multiple testing correction. For example, the agglomerative hierarchical clustering of the ARBS groups across Australian, Canadian and UK data sets was generated using the R package pvclust (68) v2.0.0 using the ward. D2 clustering method with squared Euclidean distance (100000 iterations). This package also enabled the estimation of the Approximately Unbiased Multiscale Bootstrap (AU) P-values for the Depleted group. These clustering results were confirmed by a partitional clustering approach using the R packages cluster v2.1.0 and factoextra v1.0.5.

### Classification by Ordering

The consensus ordering of events has been previously determined by estimating phylogenetic trees from the cancer cell fraction (CCF) that contained each aberration and applying the Bradley-Terry model to determine the most consistent order of events (36). There are a number of sources of uncertainty in this approach. In particular, we often cannot infer the true phylogenetic tree for each patient, and furthermore it is impossible to determine the relative timing of events on parallel branches. However, we can estimate the set of possible trees using the relative cancer cell fractions (CCFs) of the genomic aberrations involved, and from these we can estimate a set of possible orderings. Therefore, we created an algorithm where we sampled a single possible tree from the data and using this, we sampled a viable order of events for each patient. This is repeated multiple times so that the uncertainty in these estimates is encapsulated in the output distributions. Algorithms of this type are called Monte-Carlo simulations to emphasise the use of randomness in the procedure.

In this application, we adopted an extension of the Bradley-Terry model known as the Plackett-Luce model (69, 70) as the basis of our ordering analysis. The model is used to construct a probability distribution over the relative rankings of a finite set of items, the parameters of which can then be estimated from a number of individual rankings. This can be used to quantify the expected rank of each item relative to the others across the population. In our application, an item corresponds to an event, namely the emergence and fixation of a novel copy number alteration (CNA) as identified in the extracted features. Ranking these events therefore relates to the order in which they would be expected to occur. We also utilised a Plackett-Luce mixture model, which allows for subpopulations in the data with different orderings.

### The Plackett-Luce Model

Given a set of CNA occurrences for each patient with associated subclonality, we would like to infer the order in which these events generally occur. To do this we used a Plackett-Luce model, which is formulated as a ranking method, and returns a value quantifying the ranking preference. We use a different interpretation, namely the ordering, which is defined as the inverse of the ranking preference (71). Like the Bradley-Terry model, the Plackett-Luce model does not return any temporal information outside the expected order of events.

We have a set of N copy number events we are interested in:

$$C = \{ {c}_{1} , {c}_{2} , … , {c}_{N} \} \qquad ( 29 )$$

then we can apply Luce's choice axiom (69), which states that the probability of selecting one event over another from a set of events is independent of the presence or absence of the other events in the set. We can therefore write the probability of observing event i as

$$P ( {c}_{i} \vert C ) = \frac{{α}_{i}}{{\sum }_{j} {α}_{j}} \qquad ( 30 )$$

where {αᵢ} are the coefficients that quantify the relative probability of observing the i^{th} event. To reflect the ordering aspect of our application we refer to this value as the proclivity. Plackett (70) used this formalism to construct a generative model in which all N events are randomly sampled from C without replacement (i.e. a permutation). If we let Λ correspond to a permutation of the set C such that λₖ∈C and λ₁<λ₂< . . . <λ_{N}, then we write the probability density of a single ordering as

$$P ( Λ ) = {\prod }_{k}^{N} \frac{{α}_{{λ}_{k}}}{{\sum }_{j ∈ {Λ}^{( k )}} {α}_{j}} \qquad ( 31 )$$

where α_{i,k} is the proclivity associated with event λₖ, and Λ^{(k)}={λₖ, λₖ₊₁, . . . λ_{N}} is the set of possible events after k−1 events have occurred.

### Plackett-Luce Mixtures

We hypothesised that there may be more than one set of copy number orderings present in our population, and so analysing all events in one ordering scheme may not be appropriate. Furthermore, the inhibition of AR-associated breakpoints implies that some CNAs may be found more frequently with a select set of others, which is in violation of Luce's choice axiom. We therefore implemented a mixture modelling approach (71, 72), which reinstates Luce's choice axiom as the selection of each CNA can be considered as independent conditional on the mixture component. Such a finite mixture model assumes that the population consists of a number, G, of subpopulations. In this setting the probability of observing the ordering Λₛ for the s^{th} sample is

$$P ( {Λ}_{s} ) = {\sum }_{g}^{G} {ω}_{g} {P}_{g} ( {Λ}_{s} ) \qquad ( 32 )$$

where ω_{g} are the weight parameters (not to be confused with the weight matrices described above) that quantify the probability that sample s belongs to subgroup g. The appropriate parameter values can be determined using maximum likelihood estimation via an EM algorithm (72).

The number of mixture components can be chosen using the Bayesian Information Criterion (BIC) estimation, which is given by

$$BIC = N log ( M ) - 2 ℓ ( {Θ}_{ML} ) \qquad ( 33 )$$

where where Θ_{ML} is the parameter set that maximises the log-likelihood [symbol] (·), N is the number of parameters, and M is the number of samples.

The general formulation of the Plackett-Luce model takes a matrix containing the sequence of events for each patient as its input. However, we do not know the order in which these events occurred, only the presence and cancer cell fraction (CCF) of each CNA for each patient. As such, we first estimate the phylogenetic trees for each patient, and then determine the order of events from this. As we only have one tissue sample for each patient, there is often uncertainty in the tree topology and the possible sequence of events, and so we use a Monte-Carlo sampling scheme in which we sample the trees and sequence of events, and use these to estimate the distribution of possible orderings through the Plackett-Luce model. Samples with 0 or 1 CNA were not used in this analysis.

Another issue arises due to censoring, which occurs when the sample is taken before all aberrations that would occur have occurred, resulting in missing data. These are called partial-orderings in the Plackett-Luce framework, and the general approach to addressing this is to reformulate the model so that all missing events are implicitly ranked lower than the observed data (72, 73). This may not be appropriate for our analysis as we may have multiple subgroups, and we anticipate that distinct aberrations may have similar or equivalent effects in each subtype and thus will rarely co-occur despite being indicative of the same type. For instance, the absence of a very early aberration may be due to the occurrence of another less frequent aberration, so including it at the bottom of the order would bias the rankings toward more frequent aberrations. As such, our algorithm works in two phases:
1. Determine the number of mixture components and assign patients to each component,
2. Estimate the ordering profiles of each component.

These are distinct as we treat the creation of the phylogenetic trees in a slightly different way in each of these processes to account for censoring. When estimating the number of components, we calculate trees only using the observed CNAs. However, when estimating the full ordering profiles, we introduce another sampling step into our Monte-Carlo scheme where we explicitly sample a number of additional CNAs with probability proportional to the subclonality of the aberration in tumours of each mixture component. Sampling in this way reduces the bias toward more frequent aberrations.

### Assign Samples to Mixture Components

In the first phase, we
1. Sample phylogenetic trees for each patient,
2. Sample sequence of events for each patient that are consistent with trees,
3. Calculate Bayesian Information Criterion (BIC) for 1-10 mixture components,
4. Repeat steps 1-3 1000 times,
5. Determine number of mixture components which consistently had lowest BIC score,
6. Assign patients to mixture components.

The phylogenetic trees are created by initially sorting the CNAs of each patient in descending order of CCF obtained from the output of the Battenberg algorithm, iterating through them and sampling the possible parents with uniform probability. The CCF of a parent cannot be less than the sum of the CCF of their children, so viable parents are defined as ones where their CCF is greater than or equal to that of their current children plus the CCF of the CNA under consideration. The position in the sequence when the CNA occurred is sampled as any position after the parent, with uniform probability. The ordering estimates and assignment to the mixture components used the R package PLMIX as this incorporates mixture models and partial rankings (so the absence of a CNA from a sequence would not penalise its position in the ordering). A vector of assignments was retained for each sample run, and the final assignment was determined by the most frequent assignment over the course of 1000 runs.

Bayesian Information Criterion (BIC) scores were determined for each mixture component for each of the 1000 runs are shown in FIG. 8. The y-axis shows the BIC score calculated for each ordering given there are 1 to 10 mixture components as shown on the x-axis. Each individual score is shown by a cross (blue) and the mean of the scores for each component is indicated by the line (red). The BIC score was lowest for two mixture components for every sampled ordering, and so this was taken as the value to use in subsequent analysis.

### Estimate Ordering Profiles of Each Component

In the second phase, we
1. Sample phylogenetic trees for each patient,
2. Sample sequence of events for each patient that are consistent with trees,
3. Augment sequence with additional CNAs to alleviate censorship bias,
4. Calculate ordering profiles for each mixture component,
5. Repeat steps 1-5 1000 times,
6. Amalgamate results to determine final ordering profiles of each mixture component.

The phylogenetic trees and sequence of events were initially determined as before. However, instead of utilising partial rankings in the PL model, we explicitly augmented the data with additional CNAs to account for those unobserved due to censorship. The probability of a CNA being added to the sequence of events is equal to the proportion of subclonal occurrences relative to the total number of occurrences in the subpopulation defined by the mixture component. This can be written as

$$P ( {\tilde{c}}_{ig} ) = \frac{{N}_{sub} ( {c}_{ig} )}{{N}_{tot} ( {c}_{ig} )} \qquad ( 34 )$$

where N_{sub}(·) and Nₜₒₜₐₗ(·) denote the number of subclonal and total occurrences respectively of CNA cᵢ in mixture component g. As events that are predominantly subclonal have a higher chance of being unobserved due to censorship, this sampling scheme will mitigate this to a degree. Conversely, events that are predominantly clonal (i.e. early) may be unobserved due to factors other than censoring, and these have a reduced chance of being imputed.

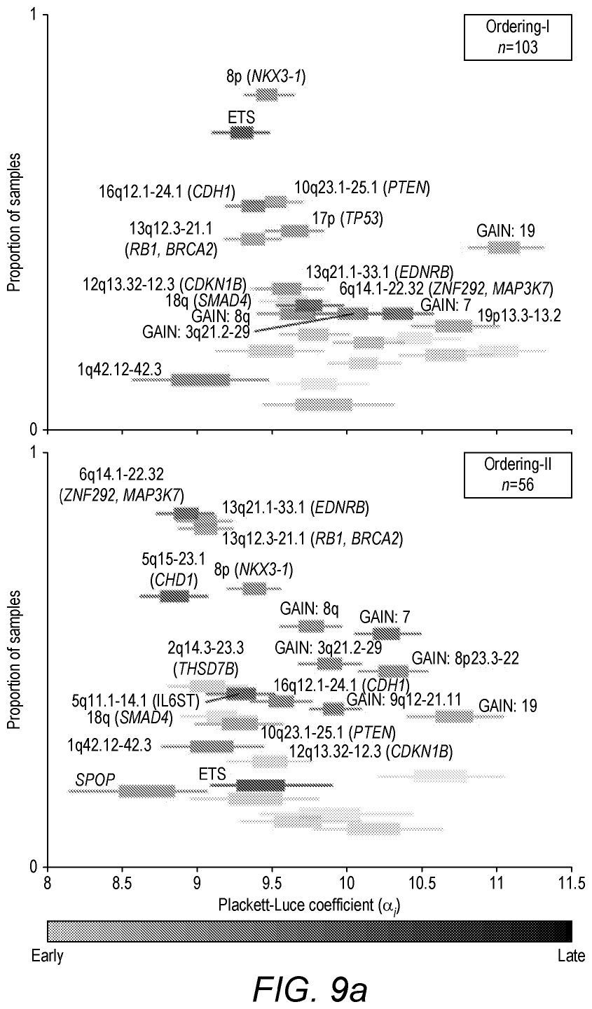

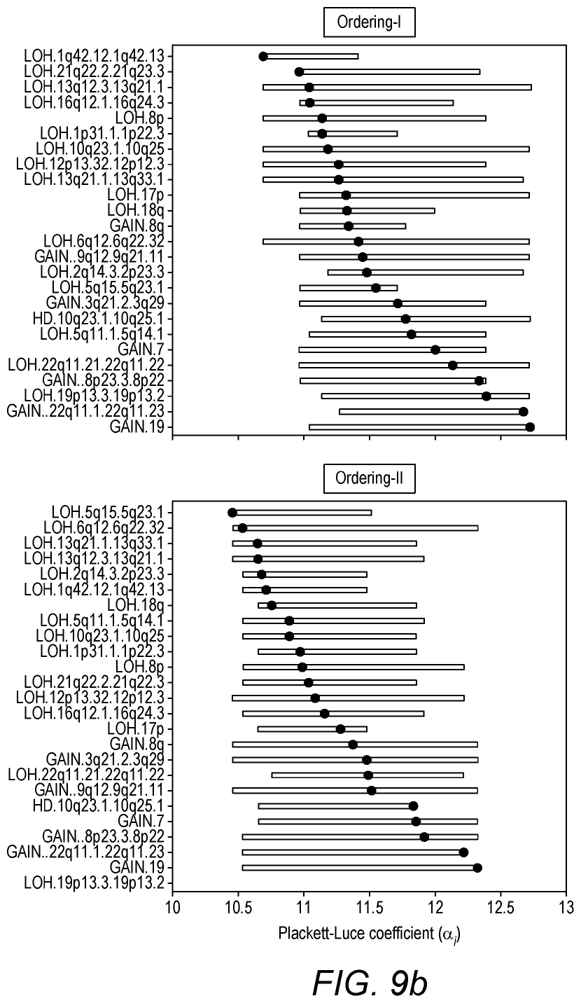

Calculating these values using the patient samples for each mixture component rather than the entire population means that only CNA subclonality relevant to each subpopulation are considered. Imputation is performed by drawing a uniform random number, r, for each patient and including the CNA in the set of additional CNAs for each patient if P(\tilde{c}_{ig})<r. The set of additional CNAs for each patient are shuffled uniformly and added to the sequence. Imputation helps to mitigate against censoring. We then calculate the ordering for each mixture component individually using the Plackett-Luce model without partial ranking. This process is repeated 1000 times and the Plackett-Luce coefficient for each CNA is calculated and used to create an empirical distribution for the Plackett-Luce coefficient for each CNA, which are used to create the box-plots in FIG. 9a.

FIG. 9a shows the proportion of the 159 samples against the Plackett-Luce coefficient for the Ordering I and Ordering II. As explained above, phylogenetic trees from individual tumours were used to estimate the two ordering profiles using a Plackett-Luce (P-L) mixture model. Tumours are assigned to Ordering-I (top) or Ordering II (bottom). The horizontal box and whisker plots (5^{th}/25^{th}/75^{th}/95^{th} percentiles) represent the bootstrap estimates of the negative Plackett-Luce coefficient αᵢ for the ith genetic alteration (x-axis). Here, the lower the value of oi, the earlier the genetic alteration is likely to occur. The y-axis shows the proportion of samples in the mixture component in which the genetic alteration was observed. Genetic alterations with a proportion above 0.25 have chromosomal regions annotated with notable driver genes in the region given in brackets. The colours of the box and whiskers denote the chromosome on which the aberration occurred.

FIG. 9a shows that the two orderings display notable differences. Tumours corresponding to Ordering-I frequently experienced an early 8p LOH (spanning NKX3.1) and ETS fusions. Less frequent LOH of regions covering the RB1, BRCA2, CDH1, TP53 or PTEN genes could also occur. This profile occasionally displayed a very early LOH of 1q42.12-42.3. Tumours corresponding to Ordering-II consistently displayed early LOH events covering MAP3K7 and 13q (EDNRB, RB1, BRCA2) and copy number gains. However, the earliest events, a mutation of the SPOP gene and LOH covering CHD1 were less frequent. Both orderings showed late gains of chromosome 19.

FIG. 9b shows the variation in the order of copy number alterations between individuals from the 159 samples. When comparing the occurrence of aberrations between individuals within each Ordering we found that the relative order of alterations was highly variable, indicating they arise stochastically. The leftmost value of each bar is the lowest Plackett-Luce (P-L) coefficient of all CNAs that must have occurred after the genetic alteration named on the left (i.e. was found to have occurred subclonally (CCF<1) when the named CNA was observed in all sampled cells (CCF=1)). The rightmost value of each bar is the highest P-L coefficient of all CNAs that must have occurred before the genetic alteration named on the left (i.e. was observed in all sampled cells (CCF=1) when the named CNA occurred subclonally). The black dots represent the P-L coefficient values of the CNA named on the left. CNAs are ordered top-to-bottom by their P-L coefficients.

### Comparison of Three Classification Methods

The table below establishes the concordance of the three classification methods described above by showing which of the 159 samples is assigned to each classification.

| | | ARBS | | | Orderings | |
|---|---|---|---|---|---|---|
| | Total | Depleted | Indeterminate | Enriched | Ordering I | Ordering II |
| MC-A | 27 | 22 | 5 | 0 | 1 | 26 |
| MC-B1 | 106 | 10 | 57 | 39 | 78 | 28 |
| MC-B2 | 26 | 0 | 12 | 14 | 24 | 2 |
| Total | 159 | 32 | 74 | 53 | 103 | 56 |

| | | ARBS | | |
|---|---|---|---|---|
| | Total | Depleted | Indeterminate | Enriched |
| Ordering I | 103 | 2 | 57 | 44 |
| Ordering II | 56 | 30 | 17 | 9 |
| Total | 159 | 32 | 74 | 53 |

The table above reveals a remarkable relationship: MC-A is a largely subset of the Depleted group (22/27), and both are almost entirely subsets of Ordering-II (26/27 and 30/32 respectively). We can therefore infer that there exists a subset of tumours that exhibit all the corresponding properties: an evolutionary trajectory (Ordering-II), a breakpoint mechanism (ARBS: Depleted) and characteristic patterns of aberrations (Metacluster: MC-A). Thus, to classify by evotype, we adopted a majority-vote approach and defined tumours that were assigned to at least two of MC-A, Depleted, or Ordering-II, as belonging to the Alternative-evotype, to distinguish them from Canonical-evotype tumours that can evolve via trajectories involving canonical AR processes.

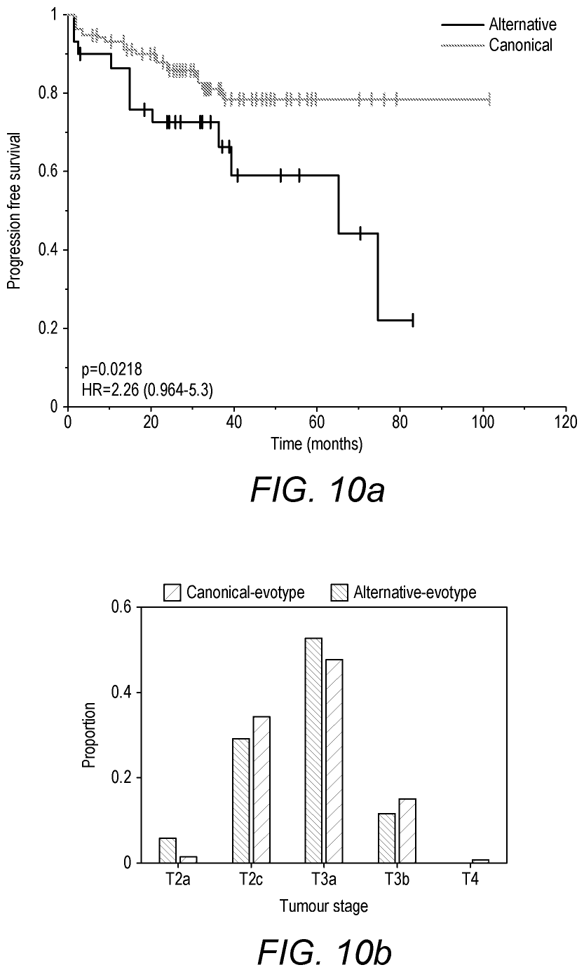

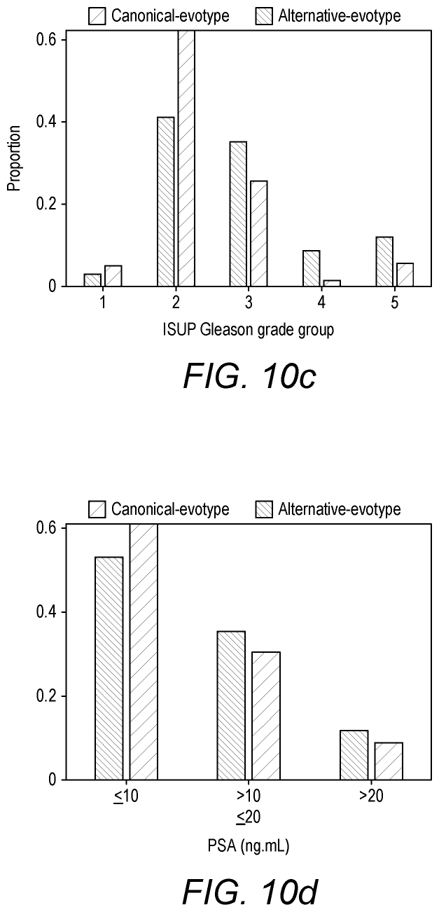

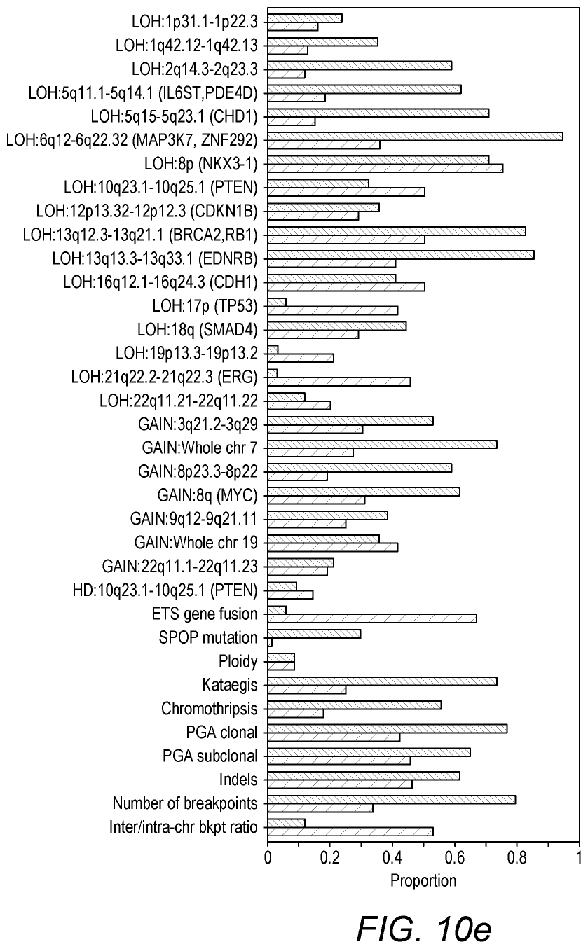

FIG. 10a plots the progression free survival against time for the patients having tumours classified as either evotype. The plot is a Kaplan-Maier plot and the P-value (0.0218) and Hazard Ratio (HR) are calculated using log-rank methods. The HR is quoted with the 5^{th}-95^{th} percentile range −2.26 (0.964-5.3). As shown patients with Alternative-evotype tumours displayed poorer prognosis. The end point is time to biochemical recurrence.

This poorer prognosis is perhaps surprising given that other clinical characteristics such as tumour stage, ISUP Gleason Grade Group and PSA (ng/ml) which are plotted in each of FIGS. 10b to 10d show that there are no observed statistically differences between the two classifications. The Chi-squared test p-value is P=0.5968 for the results of FIG. 10b, p=0.0586 for the results of FIG. 10c and P=0.191 for the results of FIG. 10d. All clinical features were taken at prostatectomy.

FIG. 10e is a bar chart showing the prevalence of each genetic aberration in each evotype. The classification of the evotype is determined using the majority consensus. The aberrations with significant differences (P<0.05 using the Fisher Exact test) between evotype are listed below (and coloured red for Alternative-evotype and blue for Canonical-evotype in the Figure). Thus, each evotype is characterised by a different propensity for certain aberrations in combination but it is noted that no single aberration was either necessary or sufficient for assignment to either evotype.

TABLE 1
Genetic aberrations associated with alternative
cancer evolutionary type (evotype)

| Chromosome region or gene | Aberration |
|---|---|
| 1q42.12-1q42.13 | Loss of heterozygosity |
| 2q14.3-2q23.3 | Loss of heterozygosity |
| 5q11.1-5q23.1 (IL6ST, PDE4D) | Loss of heterozygosity |
| 5q15-5q23.1 (CHD1) | Loss of heterozygosity |
| 6q12-6q22.32 (MAP3K7, ZNF292) | Loss of heterozygosity |
| 13q12.3-13q21.1 (BRCA2, RB1) | Loss of heterozygosity |
| 13q13.3-13q33.1 (EDNRB) | Loss of heterozygosity |
| 3q21.2-3q29 | Gain |
| Chromosome 7 | Gain |
| 8p23.3-8p22 | Gain |
| 8q (MYC) | Gain |
| SPOP | Mutation |
| Kataegis | Present |
| Chromothripsis | Present |
| PGA clonal | Present |

TABLE 2
Genetic aberrations associated with canonical cancer evotype

| Chromosome region or (gene) | Aberration |
|---|---|
| 17p (TP53) | Loss of heterozygosity |
| 19p13.3-19p13.2 | Loss of heterozygosity |
| 21q22.2-21q22.3 (ERG) | Loss of heterozygosity |
| ETS | Gene fusion |
| Number of breakpoints | High |
| Inter/intra chromosomal breakpoint ratio | High |

### Statistical Model of Evotype Convergence

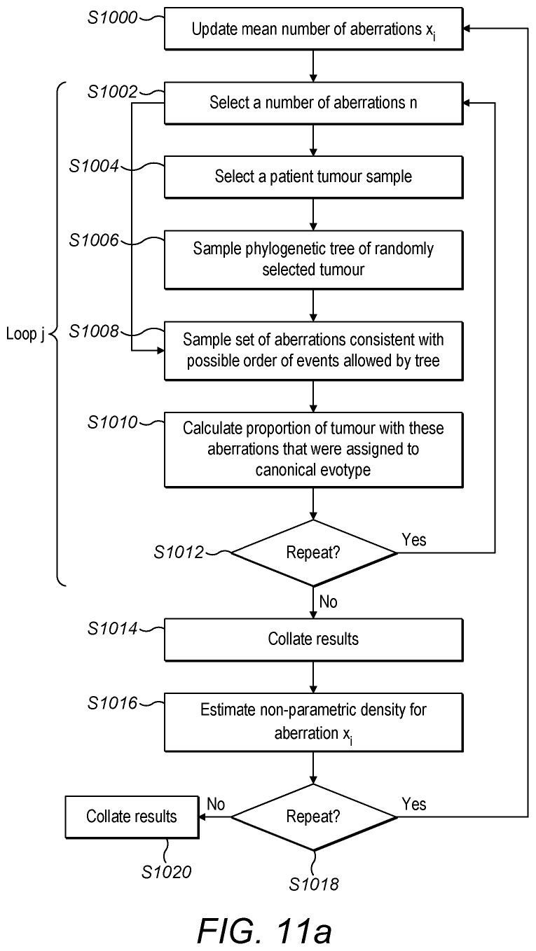

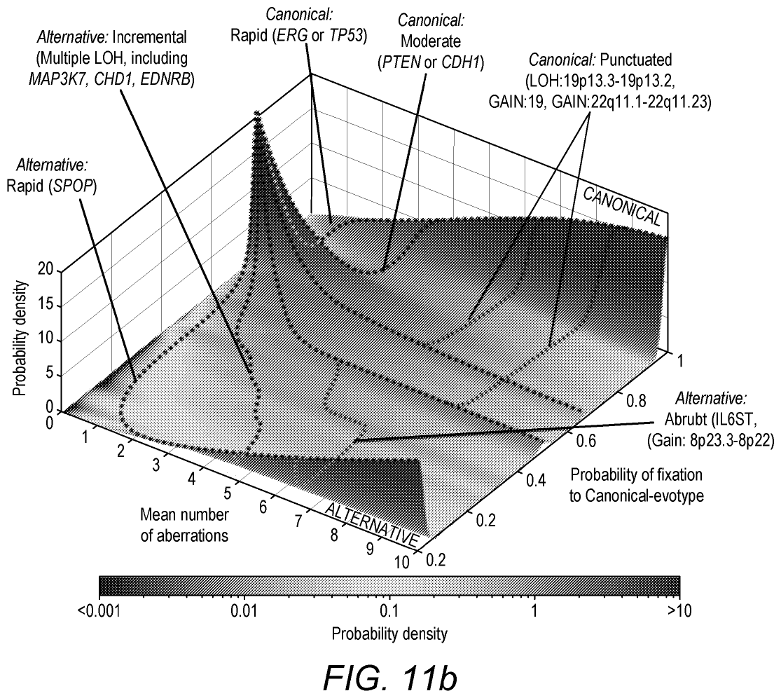

FIG. 11a is a flowchart of a statistical algorithm for obtaining the probability of convergence to the Canonical or Alternative evotypes based on accumulation of genetic alterations. An example output from this algorithm is shown in FIG. 11b. We assume that the accumulation of such aberrations in each individual tumour followed a stochastic process in which the order and relative timing of the aberrations occurred with some degree of randomness/stochasticity. Similar to the Ordering analysis (described above), we utilised a statistical algorithm in which we simulated a number of possible aberrations consistent with the possible phylogenetic trees, and then estimated the probability that tumours with these aberrations converged to the Canonical-evotype (the probability of convergence to the Alternative-evotype is 1 minus the probability of convergence to the Canonical-evotype). The algorithm iterates through an increasing number of aberrations (Loop i), performing several Monte-Carlo repeats of ordering samples (Loop j).

The accumulation of aberrations in a tumour is modelled as a Poisson process (74). FIG. 11a shows that the first step in each iteration of the first loop i is to update the mean number xᵢ of aberrations across all patients at the ith iteration (step S1000). This is then used as the input parameter to a Poisson random number generator to draw the number of aberrations to be sampled, n, in each iteration of Loop j. In other words, the number n of aberrations which is going to be considered in this iteration is sampled at random (step S1002).

We then identified those tumours with sufficient aberrations and selected one with uniform probability (step S1004). The data for the selected tumour is then used to sample a phylogenetic tree using the relative CCFs of the aberrations (step S1006). The phylogenetic tree is sampled from the aberration data. We then used the phylogenetic tree to sample an order of occurrence for the aberrations (step S1008), and retained the first n (thus as illustrated by the connecting arrow, the output from step S1002 is used in this step). In other words, using both n and the phylogenetic tree obtained from the previous step, the set of aberrations which are consistent with the possible order of events allowed by the phylogenetic tree are sampled. The set of aberrations generated in this step may be termed Aⱼ={a₁, a₂, . . . , aₙ}, The aberrations used were the SPOP mutations and the CNAs identified in the feature extraction; inter-intra chromosomal breakpoints, ETS status and chromothripsis are not included as these do not have associated CCFs and therefore cannot be used to determine the order of events.

The sampled set of aberrations is then used to calculate the proportion of tumours with these aberrations that have been classified as the Canonical evotype (step S1010). The calculated proportion may be termed the probability pⱼ of tumours with aberrations Aⱼ being assigned to the Canonical evotype. The aberration data is used to perform this calculation. For a set of sampled aberrations, Aⱼ={a₁, a₂, . . . , aₙ}, we identified the patients for which Aⱼ⊆Pₖ, where Pₖ denotes the full set of aberrations present in patient k. We can then identify which of these were assigned to the Canonical-evotype. We can now calculate the probabilities

$$p ( {A}_{j} ) = \frac{N ( {A}_{j} ⊆ {P}_{k} )}{N ( {P}_{k} )} , \qquad ( 35 )$$

$$p ( Canonical ⋂ {A}_{j} ) = \frac{N ( Canonical ⋂ ( {A}_{j} ⊆ {P}_{k} ) )}{N ( {P}_{k} )} \qquad ( 36 )$$

where N(·) denotes the number of tumours that obey the condition in brackets. We can now calculate the conditional probability

$$p ( Canonical \vert {A}_{j} ) = \frac{N ( Canonical ⋂ {A}_{j} )}{p ( {A}_{j} )} \qquad ( 37 )$$

The final step in each inner loop (Monte Carlo loop) is to determine whether further iterations are to be carried out (step S1012). If a further iteration is to be performed, the method loops back to the step S1002 of randomly selecting a number of aberrations and steps S1004, S1006, S1008 and S1010 are repeated.

If no further repetitions of the inner loop are to be performed, i.e. if no further samples are to be considered, the results which have been obtained so far are collated (step S1014). The results may be collated as a set of probabilities sᵢ=[p₁, p₂, . . . , pⱼ] for all the selected samples of the mean number of aberrations xᵢ with p₁ being the probability calculated in first iteration and so on until the jth iteration is completed. The collated set of probabilities is used to obtain a non-parametric density estimation (step S1016) where pdf (Canonical|xᵢ) is the probability density function of tumours being assigned to the Canonical evotype for the mean number of aberrations xᵢ. Thus, the values of sᵢ are passed into a nonparametric density estimation scheme using Gaussian kernels with bandwidth 0.025. As we are estimating the probability density function of a set of probabilities, which are bound at [0, 1], we ensured support only over this interval using the reflection method (75).

In this example, we performed 100,000 samples and thus obtained 100,000 values for each p(Canonical|Aⱼ). The next step is to determine whether further iterations of the outer loop i are to be carried out (step S1018). The outer loop i is repeated for each number of mean aberrations, for example for xᵢ∈{0,0.01,0.02, . . . ,10}; i∈1,2, . . . ,1000. If all iterations have not yet been completed, the method loops back to the step S1000 of updating the mean number of aberrations at the first step S1000, the inner loop j is then repeated. If no further repetitions of the outer loop are to be performed, the results which have been obtained are collated (step S1020).

In summary, loop i iterates through an increasing number of mean aberrations and loop j performs multiple samples, selecting a patient at random and samples an order of events for consistency with the possible phylogenetic trees and current number of mean aberrations. The samples are collated and used to estimate a probability density function for each mean number of aberrations.

FIG. 11b shows an example output from the algorithm of FIG. 11a generated using the data from the 159 samples as previously described. FIG. 11b is a surface plot showing the probability density of a tumour being assigned to the Canonical evotype relative to the number of aberrations. As individual evolutionary trajectories involve the stochastic accumulation of multiple genomic aberrations, is it impossible to specify each evolutionary route. However, linking regions of high density as the number of aberrations increased can indicate common modes of evolutionary progress. In other words, we can determine common modes of evolution by tracking the genetic alterations prevalent in tumours at the point of convergence to either evotype in our model. Through this we can identify paths in the probability density surface plot that correspond to the accumulation of these genetic alterations.

Initially the probability density is concentrated at ˜0.78, the proportion of Canonical-evotype tumours in our sample set. As the number of aberrations increases, the density diverges to accumulate at 1 (corresponding to unambiguous assignment to the Canonical-evotype) and 0 (Alternative-evotype). An individual tumour will follow a trajectory through this probability landscape dependent on the type and order of aberrations, favouring areas of high probability density that need not be adjacent. Examples of such routes (or paths) are illustrated by the black dashed lines in FIG. 11b. These include: Canonical: Rapid; Canonical: Moderate; Canonical: Punctuated; Alternative: Rapid; and Alternative: Incremental. There are also two Equilibrium routes which include LOH in NKX3.1 or IL6ST or LOH in RB1 and BRCA2. The labels include their likely evotype, a behavioural description and the notable driver genes affected by aberrations that are prevalent in the areas along the path.

Canonical: Rapid is indicated by early TP53 loss or ERG gene fusion fixation which lead to the Canonical-evotype. Alternatively, loss of regions covering PTEN or CDH1 can coerce progression toward the Canonical-evotype and this evolutionary trajectory is termed Canonical: Moderate. For the Canonical-evotype, there were a number of aberrations that were often the last step in convergence, particularly LOH of 19p13.3-19p13.2, and gains of chromosome 19 and region 22q11.1-22q11.23 and this trajectory is termed Canonical: Punctuated.

When an SPOP mutation occurs first, it confers high probability (˜0.91) of progression to the Alternative-evotype and this is termed Alternative: Rapid. Other routes to the Alternative-evotype involve the accumulation of multiple individual LOH events involving genes such as MAP3K7, CHD1 or EDNRB in any order. This trajectory is termed Alternative: Incremental. LOH of IL6ST or gain of region 8p23.3-8p22 strongly influenced convergence after a number of aberrations had already accumulated and is termed Alternative: Abrupt.

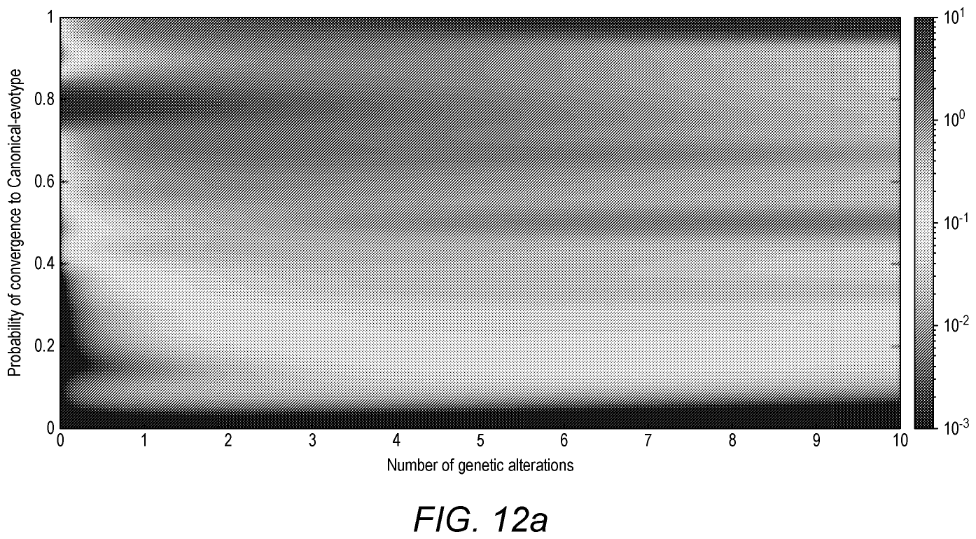

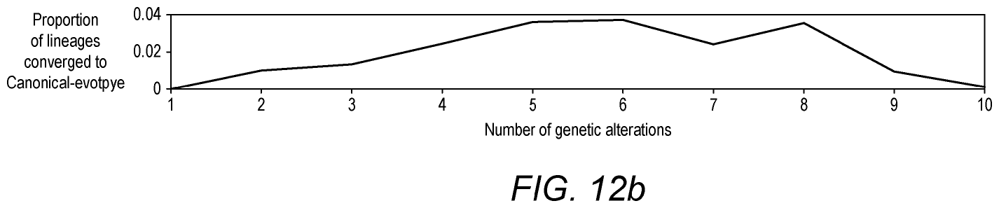

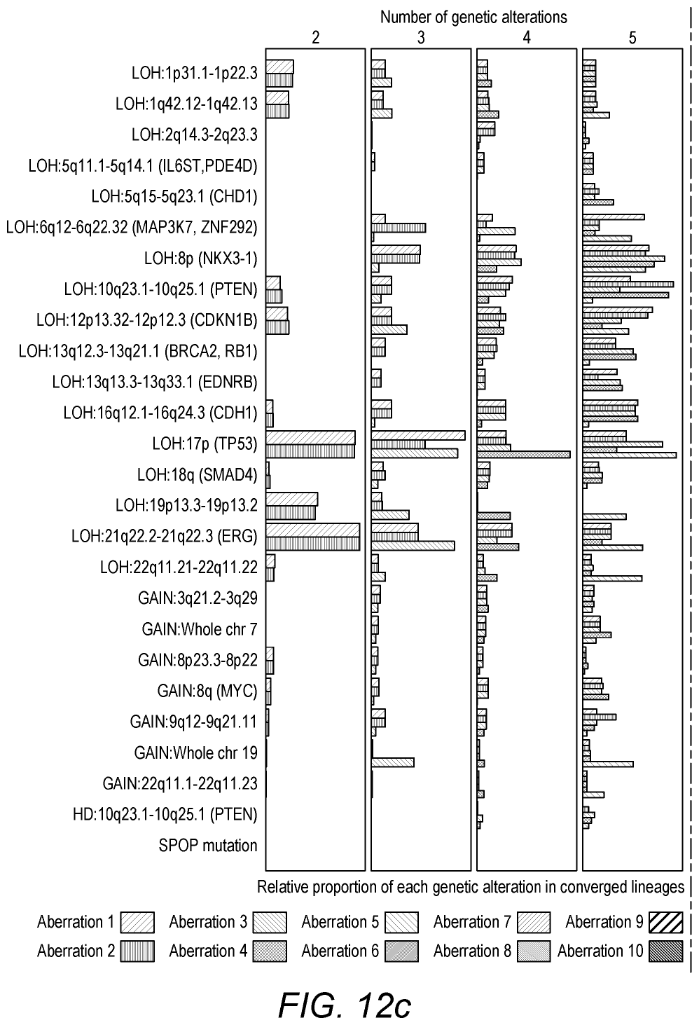

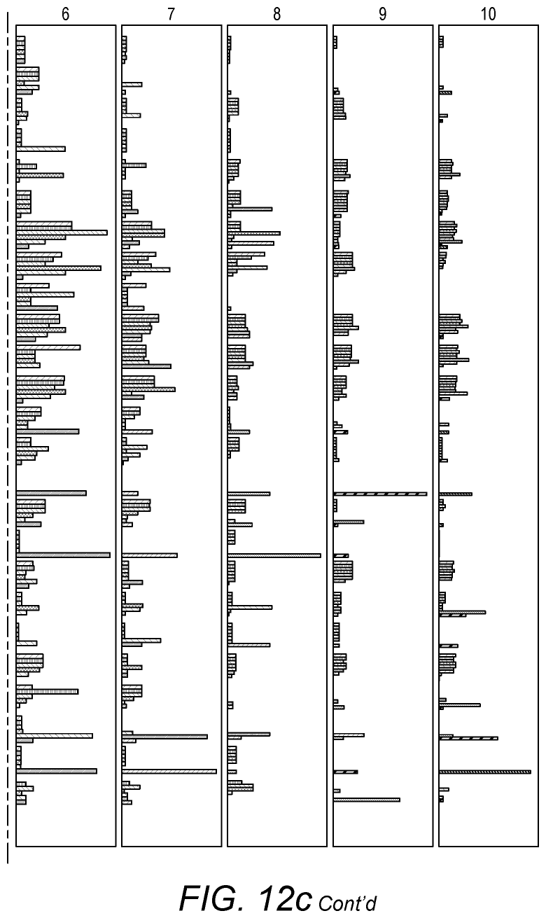

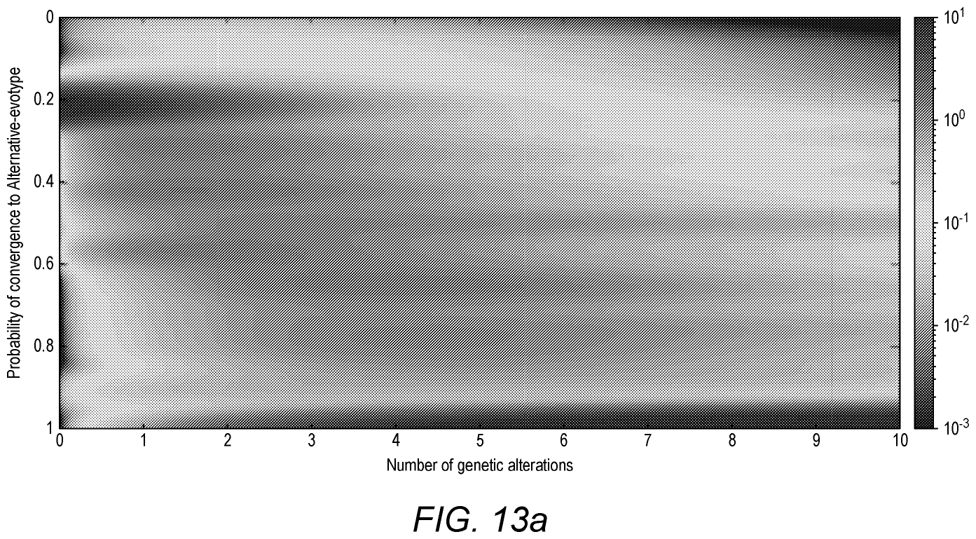

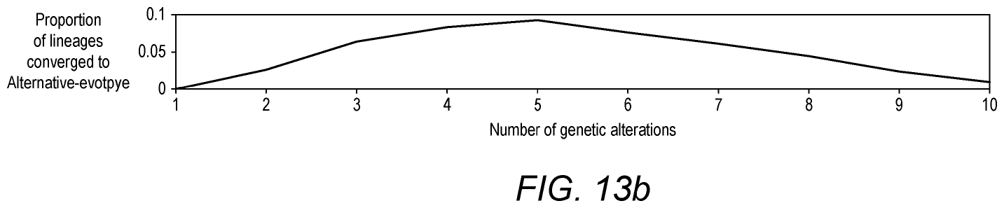

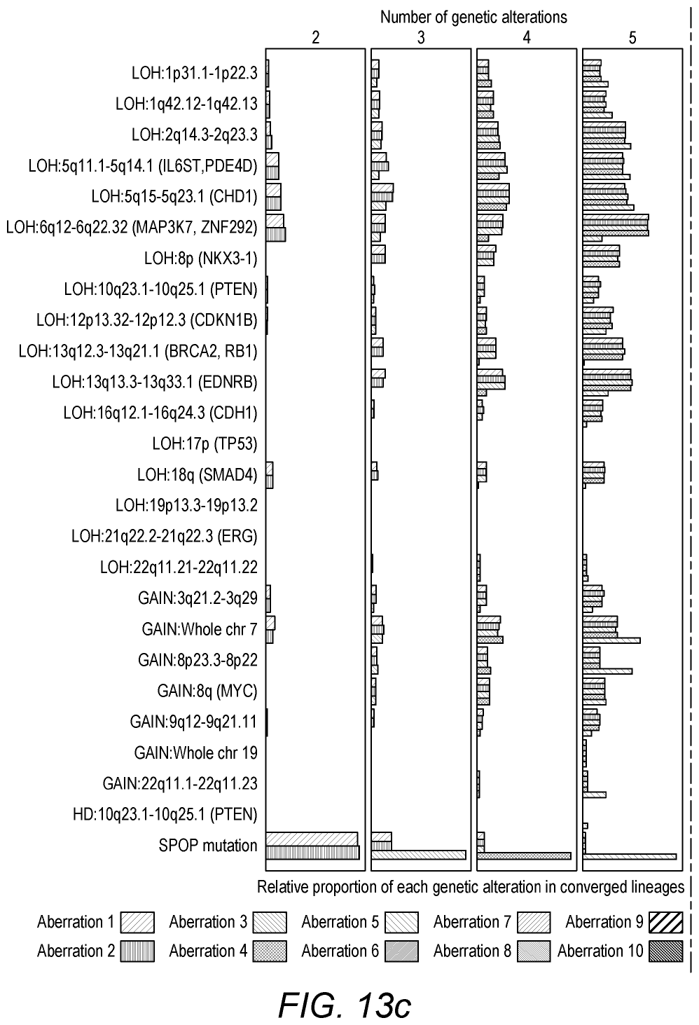

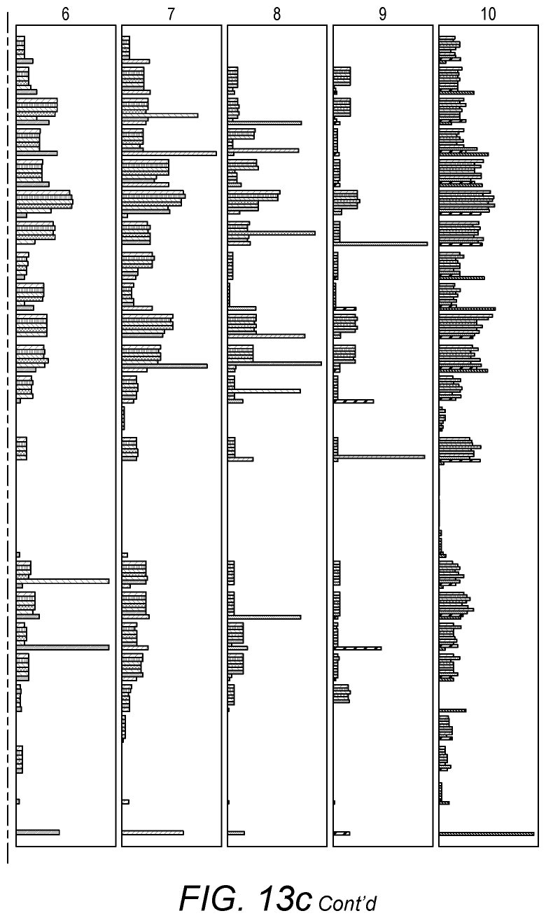

In other words, the model simulations from FIG. 11a may be used to investigate the common evolutionary trajectories involved in convergence to each evotype (black dashed lines in FIG. 11b). As shown in more detail in FIGS. 12a to 13c, the aberrations that characterise the common evolutionary process may be investigated further. In the modelling process, we recorded the order of genetic alterations for each of the trajectories used to calculate the pdf. We extracted each trajectory that had converged to the Canonical or Alternative evotypes (i.e. had a p(Canonical|Aⱼ)=0 or 1 and assigned these into sets by the number of genetic alterations in the trajectories i.e. {A₁}, {A₂}, . . . , {A₁₀}. We then ran a filtering step for each set where we removed any trajectories that had occurred in sets corresponding to fewer genetic alterations, meaning we were left with trajectories that only converged to either evotype with the final genetic alteration for each set. We can then identify the position and frequency of occurrence of each genetic alteration in each set. Using this information, we can calculate the pdf values for frequent combinations of genetic alterations in order, and use these to create the representative paths through the probability density (black dashed lines; shown in FIG. 11b).

FIG. 12a is a 2D surface plot showing the probability density of all Canonical-evotype tumours being assigned to the Canonical-evotype as the number of aberrations increase. FIG. 12b is a graph showing the proportion of lineages that converged to the Canonical-evotype at each number of genetic alterations. FIG. 12c is a bar plot showing the relative proportion of genetic alterations and the position in which they occurred for the lineages which converged to the Canonical-evotype. The bar plot shows the relative proportions for each number of genetic alterations (e.g. 2, 3, . . . , 10). For example, for two genetic alterations the relative proportions of each of the first and second alterations are shown and the largest proportions are shown for TP53 and ERG.

FIG. 13a is a 2D surface plot showing the probability density of all Alternative-evotype tumours being assigned to the Alternative-evotype as the number of aberrations increase. FIG. 13b is a graph showing the proportion of lineages that converged to the Alternative-evotype at each number of genetic alterations. FIG. 13c is a bar plot showing the relative proportion of genetic alterations and the position in which they occurred for the lineages which converged to the Alternative-evotype. The bar plot shows the relative proportions for each number of genetic alterations (e.g. 2, 3, . . . , 10). For example, for two genetic alterations the relative proportions of each of the first and second alterations are shown and the largest proportions occur at SPOP.

Taken together, the findings described above reveal prostate cancer disease types that arise as a result of different trajectories of a stochastic evolutionary process in which different alterations can tip the balance toward either outcome. The definition of evotypes provides additional context to relationships between individual aberrations reported in previous studies. Co-occurring aberrations that have been identified previously can be related to particular evotypes. For the Canonical-evotype, this includes LOH events affecting PTEN and CDH1 (20), or PTEN and TP53 (21). Conversely, CHD1 losses have previously been observed in conjunction with SPOP mutations (22, 23), as has LOH affecting MAP3K7 (24) and 2q22 (25); all these aberrations are associated with the Alternative-evotype.

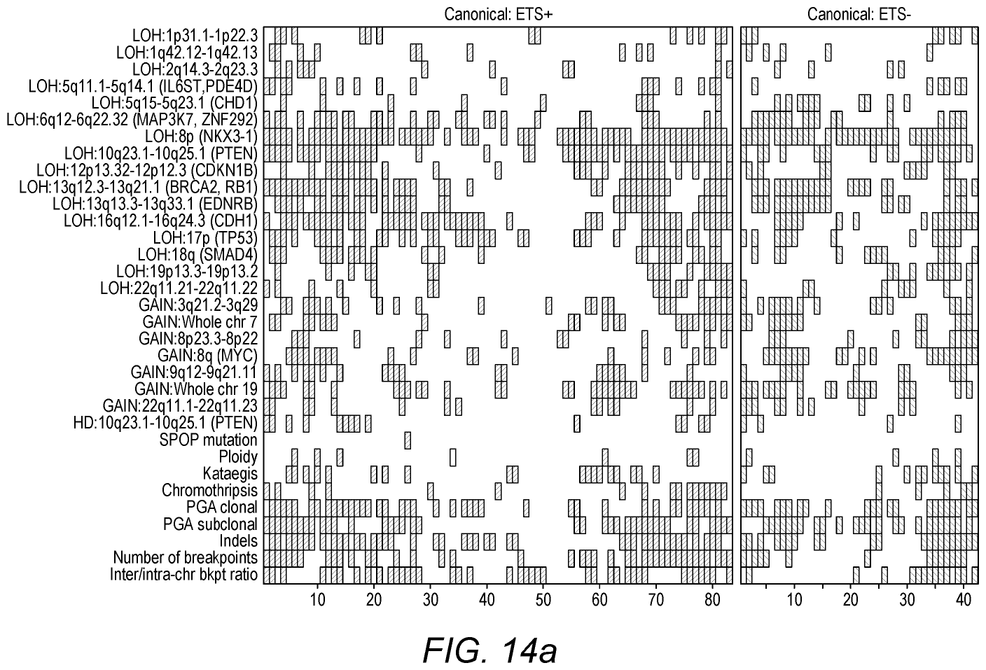

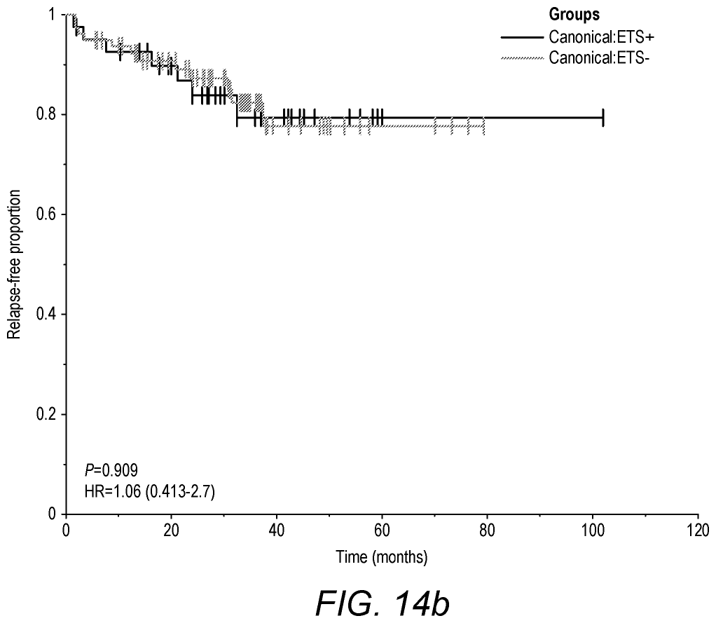

The most widely used basis for genomic prostate cancer subtyping is the ETS status, where tumours are classified by the presence or absence of an ETS fusion into ETS+ and ETS− respectively (7, 8, 10, 11). FIGS. 14a and 14b illustrate some comparative data for the methods described above and ETS data. Regarding the Alternative-evotype tumours, 94% were ETS−. Moreover, alterations such as SPOP mutations and CHD1 LOH that are characteristic of this evotype have previously been associated with the ETS− subtype (10, 26). By contrast, there is a relatively even balance of ETS− and ETS+ tumours for the Canonical-evotype tumours.

FIG. 14a shows the aberrations present in the Canonical-evotype tumours when split into ETS− (n=42 or 44%) and ETS+ (n=83 or 66%). Continuous variables were converted into binary by setting those greater than or equal to the median to 1 and those less than the median to zero. Samples were ordered by hierarchical clustering with Hamming distance means. No aberration was significantly associated with either ETS group (Q>0.05, Fisher exact). FIG. 14b shows the Kaplan-Meier plot for ETS+ and ETS− tumours that were assigned to the Canonical-evotype. The P-value (0.909) and Hazard Ratio (1.06 (0.413-2.7) were calculated using log-rank methods and the HR is quoted with its 5^{th} to 95^{th} percentile ranges. The end point in FIG. 14b is time to biochemical recurrence. As shown in these Figures, there were no significant differences in risk or prevalence of any of the genomic features between ETS+ and ETS− tumours of the Canonical-evotype which is consistent with its definition as a distinct disease type.

### Application of Evotypes to Classification of New Tumours

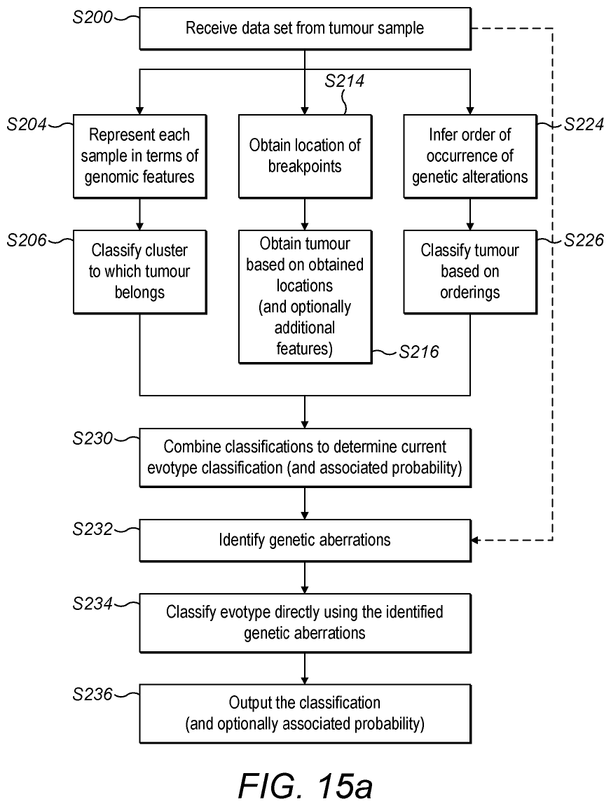

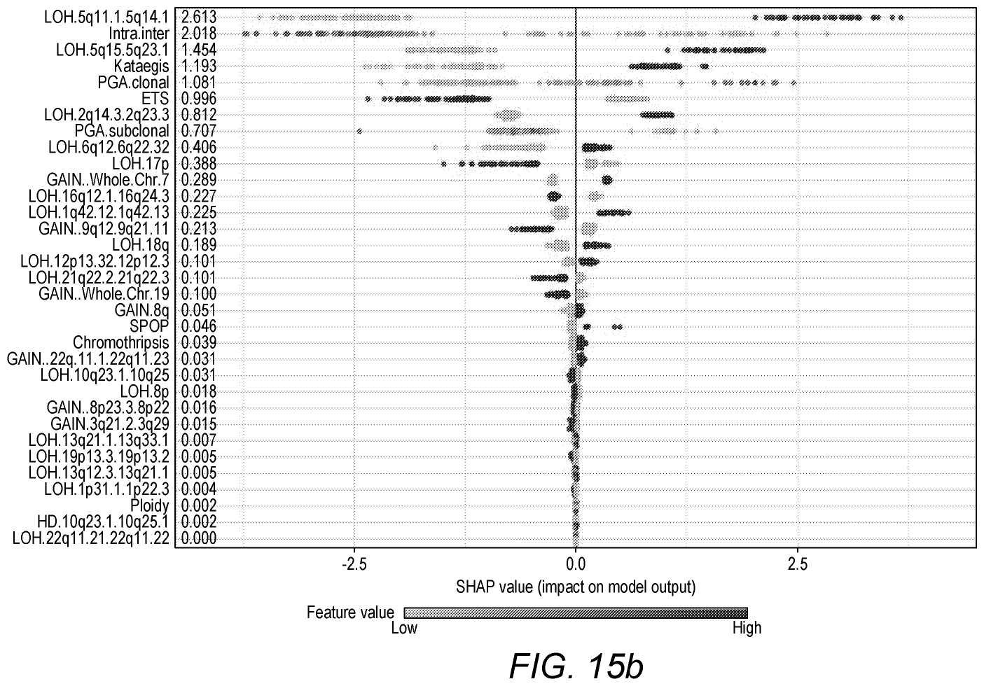

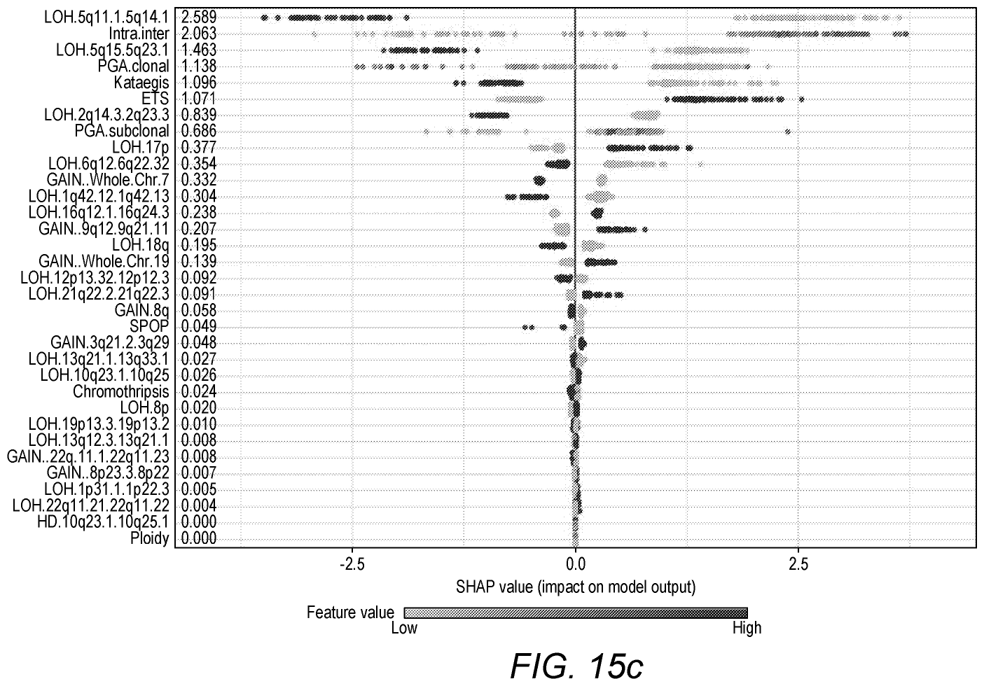

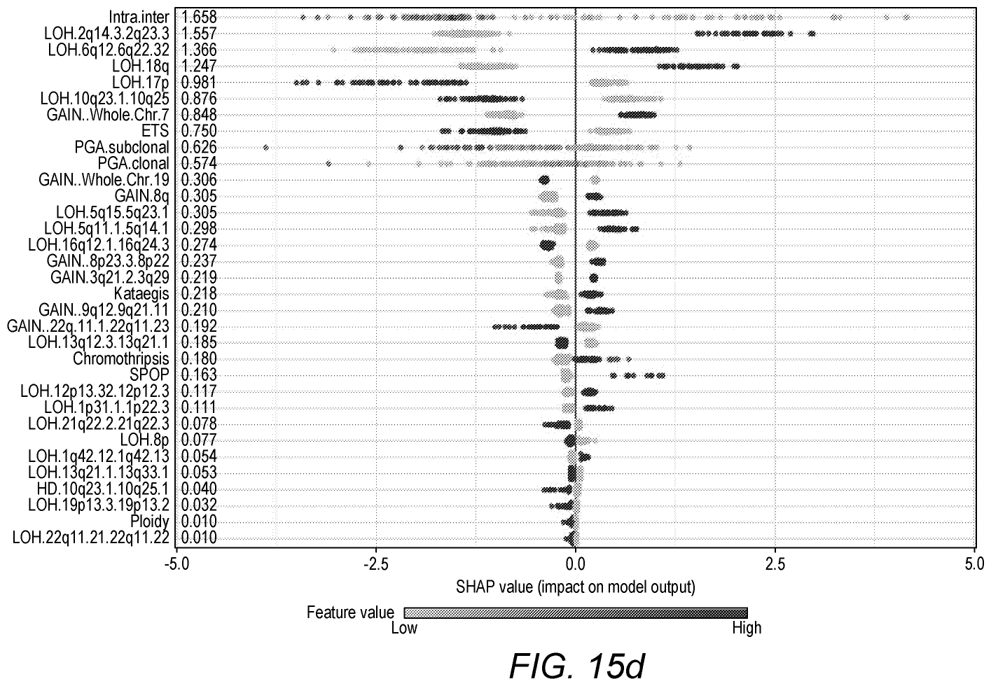

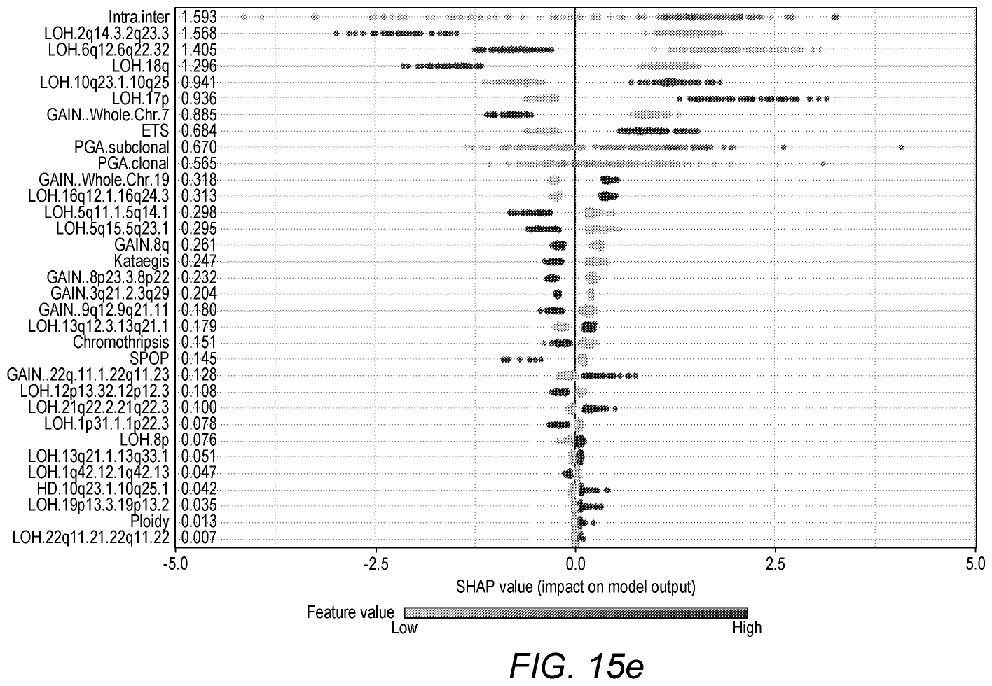

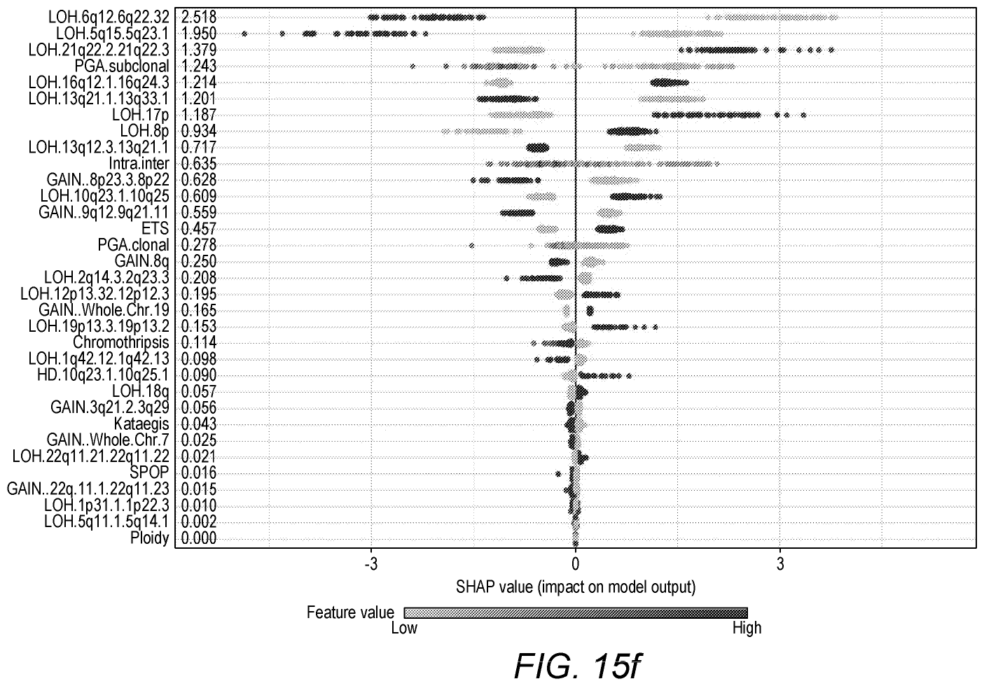

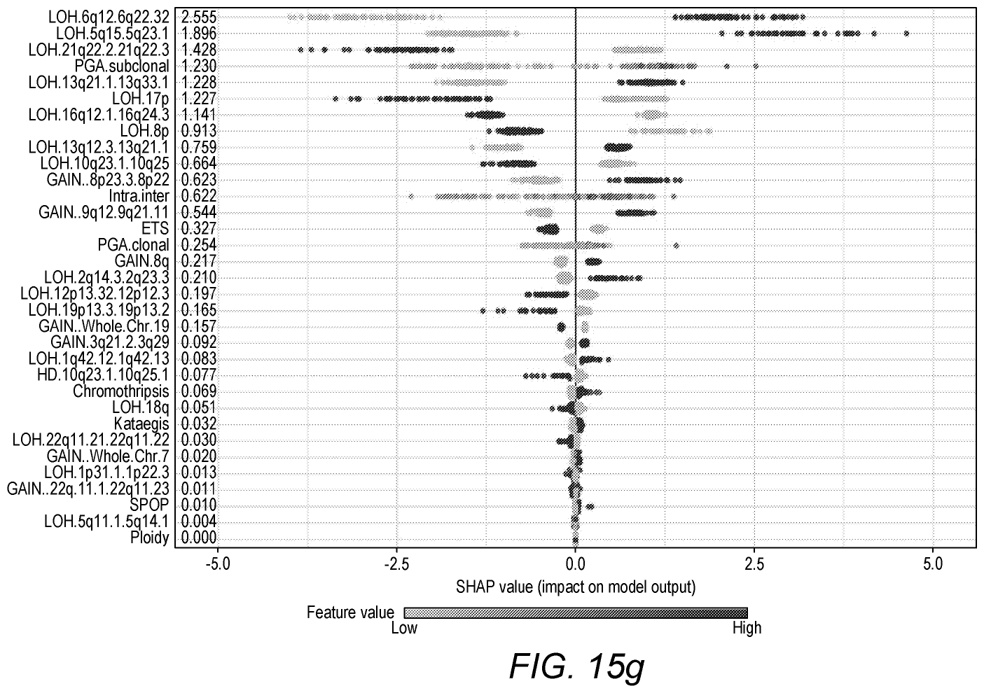

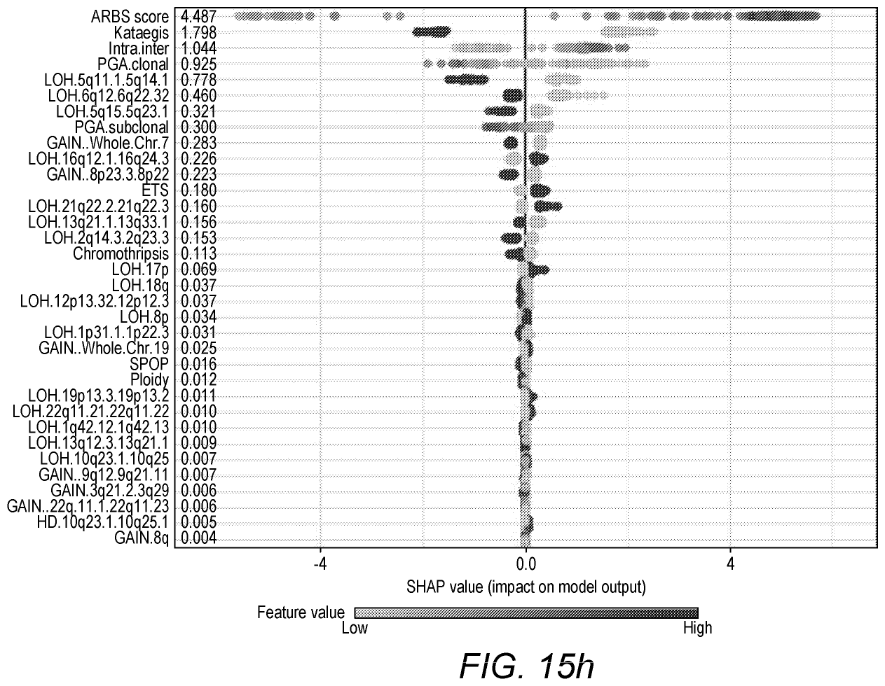

Now that the presence of evolutionary disease types is established, we can classify the metaclusters and even the evotypes directly from the feature set using classification methods such as, but not limited to, neural networks, random forests and boosted decision trees. FIG. 15a illustrates a possible method for classifying tumours. In a first step, a data set is received (step S200). FIG. 15a shows that three distinct classifications may be done based on the received data set. The classifications are a clustering classification based on clustering, an ARBS classification based on the proximity of DNA breakpoints to androgen receptor binding sites (ARBS) and an ordering classification based on ordering of events. These classifications may be performed in parallel or sequentially. Although in preferred arrangements, all three classifications are carried out, the overall classification of the tumour may be based on one, two or three of the classifications (which may be termed intermediate classifications). The received data needs to be relevant to the method of classification being used. For example, for classifying based on cluster, gene sequencing may be used to extract the relevant information.

When using the metacluster classification shown in the first branch, a trained neural network can be used to process the raw inputs from new samples to generate the feature representation, and this can be used for assignment to one of the metaclusters. Alternatively, we can use the raw inputs (or subset thereof) to develop a simple ML classifier that classifies by metacluster directly. For example, the SHapley Additive explanation (SHAP) value may be used to quantify the relative importance of the features when performing the classification using the gradient boosted decision tree method XGBoost. A value of zero indicates that the feature is not necessary to perform classification. FIG. 15b illustrates the SHAP value for each feature when classifying a sample as belonging to Metacluster A and FIG. 15c illustrates the SHAP value for each feature when classifying a sample as belonging to Metacluster B. When generating these Figures, the ARBS score has been omitted as a feature for consistency with the other Figures below.

For each of FIGS. 15b and 15c, each feature is ranked by its feature value and not unsurprisingly, there is a similar ranking in each Figure. Each feature with a high positive ranking for Metacluster A has a high negative ranking for Metacluster B and vice versa and the positive values show that the features are strongly suggestive of belonging to a particular Metacluster: Features for clustering classification—Metacluster A

| Features | Metacluster A | Shap Value |
|---|---|---|
| Loss of heterozygosity: 5q11.1-5q14.1(IL6ST, | High Positive | 2.613 |
| PDE4D) | | |
| Loss of heterozygosity: 5q15-5q23 (CHD1) | High Positive | 1.454 |
| Kataegis | High Positive | 1.193 |
| Percentage Genome Altered (clonal component) | Med Positive | 1.081 |
| Loss of heterozygosity: 2q14.3-2q23.3 | High Positive | 0.812 |
| Loss of heterozygosity: 6q12-6q22.32 (MAP3K7, | High Positive | 0.406 |
| ZNF292) | | |
| Gain of whole chromosome 7 | High Positive | 0.289 |
| Loss of heterozygosity: 1q42.12.1-1q42.13 | High Positive | 0.225 |
| Loss of heterozygosity: 18q | High Positive | 0.189 |
| Loss of heterozygosity: 12p12.32-12p12.3 | High Positive | 0.101 |
| Gain: 8q (MYC) | High Positive | 0.051 |
| SPOP | High Positive | 0.046 |
| Chromothripsis | High Positive | 0.039 |
| Gain: 22q11.1-22q11.23 | High Positive | 0.031 |
| LOH: 13q21.1-13q33.1 | High Positive | 0.007 |

### Features for Clustering Classification—Metacluster B

| Features | Metacluster B | Shap Value |
|---|---|---|
| Ratio of intra- to inter- chromosomal chained structural | Med Positive | 2.063 |
| variants | | |
| ETS | High Positive | 1.071 |
| Percentage Genome Altered (subclonal component) | Med Positive | 0.686 |
| Loss of heterozygosity: 17p | High Positive | 0.377 |
| Loss of heterozygosity: 16q12.1-16q24.3 | High Positive | 0.238 |
| Gain: 9q12.9-9q21.11 | High Positive | 0.207 |
| Gain of whole chromosome 19 | High Positive | 0.139 |
| Loss of heterozygosity: 21q22.2-21q22.3 | High Positive | 0.091 |

In a first step, the sample may optionally be represented in terms of genomic features (S204), e.g. the features identified above. The tumour can then be classified into a specific cluster (S206). We can achieve 95.60% accuracy in distinguishing Metacluster 1 (MC-A) from Metaclusters 2 (MC-B1) and 3 (MC-B2).

Returning to FIG. 15a, the next classification which is illustrated is the ARBS classification, the first step is to obtain the location of the DNA breakpoints (step S214) relative to androgen receptor binding sites (ARBS) from the input data. To classify the tumour, the proximity of the obtained locations to ARBS was compared to the proximity of locations within an expected distribution to ARBS to determine an ARBS score. The expected distribution of break points may be termed a base line distribution. Classes (enriched, depleted or indeterminate) were determined (step S214) based on whether the tumour displayed more proximal breakpoints than expected (enriched), fewer proximal breakpoints than expected (depleted) or no statistically significant difference (indeterminate). The baseline distribution may be defined as the distribution that would be expected if the DNA breakpoints were distributed uniformly across the genome. Alternatively, another baseline distribution may be used.

As an example, a baseline (or expected) distribution may include permuted data which may be generated by simulating 1000 data sets in which the DNA breakpoint positions were permuted to new positions in the genome with a uniform distribution. The distance to the closest ARBS was calculated for each simulated breakpoint in each data set. Similarly, the distance to the closest ARBS was calculated for each observed or obtained breakpoint. A double stranded DNA break may be considered to be relatively proximal to an ARBS when the break is less than a threshold number of base pairs (e.g. 20,000 bps) from an ARBS. The ARBS score may be calculated by normalising the number of relatively proximal DNA breaks by the number of proximal breakpoints expected by chance. The proportion of breakpoint positions which are relatively proximal may thus be calculated for both the observed data (B_obs) and the permuted data (B_perm). If the observed proportion of breakpoints which are relatively proximal (B_obs) is above an upper threshold (e.g. the 97.5% th percentile) of the proportion of breakpoints in the permuted data which are relatively proximal (B_perm), i.e. B_obs>P_97.5% (B_perm), the tumour is classified as Enriched. In other words, if the ARBS score is above an upper threshold (e.g. 97.5%), the tumour is classified as Enriched. If the observed proportion of relatively proximal breakpoints (B_obs) is below a lower threshold (e.g. the 2.5% th percentile) of the proportion of relatively proximal breakpoints in the permuted data (B_perm), B_{obs}<P_{2.5%}(Bₚₑᵣₘ), the tumour is classified as Depleted. In other words, if the ARBS score is above a lower threshold (e.g. 2.5%), the tumour is classified as Depleted. Otherwise the difference is not significant, and the tumour is classified as indeterminate.

When using the ARBS classification shown in the second branch, a feature representation can be used alongside the ARBS score itself to assign each sample to one of the two classifications: enriched or depleted. The SHapley Additive explanation (SHAP) value may be used to quantify the relative importance of the features when performing the classification using the gradient boosted decision tree method XGBoost. FIG. 15d illustrates the SHAP value for each feature when classifying a sample as belonging to a depleted tumour and FIG. 15e illustrates the SHAP value for each feature when classifying a sample as belonging to an enriched tumour. When generating these Figures, the ARBS score has been omitted as a feature for consistency with the other Figures.

For each of FIGS. 15d and 15e, each feature is ranked by its feature value and not unsurprisingly, there is a similar ranking in each Figure. Each feature with a high positive ranking for a depleted tumour has a high negative ranking for an enriched tumour and vice versa and the positive values show that the features are strongly suggestive of belonging to a particular type of tumour. There are the following features with a SHAP value of about 2 or more:

### Features for Enriched Classification:

| | Impact on | Shap |
|---|---|---|
| Features | model | Value |
| Ratio of intra- to inter- chromosomal chained structural | Med Positive | 1.593 |
| variants | | |
| Loss of heterozygosity: 10q23.1-10q25 | High Positive | 0.941 |
| Loss of heterozygosity: 17p | High Positive | 0.936 |
| ETS | High Positive | 0.684 |
| Percentage Genome Altered (subclonal component) | Med Positive | 0.670 |
| Percentage Genome Altered (clonal component) | Med Positive | 0.565 |
| Gain of whole chromosome 19 | High Positive | 0.318 |
| Loss of heterozygosity: 16q12.1-16q24.3 | High Positive | 0.313 |

### Features for Depleted Classification

| Features | Impact on model | Shap Value |
|---|---|---|
| Loss of heterozygosity: 2q14.3-2q23.3 | High Positive | 1.557 |
| Loss of heterozygosity: 6q12-6q22.32 (MAP3K7, ZNF292) | High Positive | 1.366 |
| Loss of heterozygosity: 18q | High Positive | 1.247 |
| Gain of whole chromosome 7 | High Positive | 0.848 |
| Gain: 8q | High Positive | 0.305 |
| Loss of heterozygosity: 5q15-5q23 | High Positive | 0.305 |
| Loss of heterozygosity: 5q11.1-5q14.1 (IL6ST, PDE4D) | High Positive | 0.298 |
| Gain: 8p23.3-8p22 | High Positive | 0.237 |
| Gain: 3q21.2-3q29 | High Positive | 0.219 |
| Kataegis | High Positive | 0.218 |
| Gain: 9q12.9-9q21.11 | High Positive | 0.210 |
| Chromothripsis | High Positive | 0.210 |
| SPOP | High Positive | 0.180 |
| LOH: 12p12.32-12p12.3 | High Positive | 0.117 |
| LOH: 1p31.1-1p22.3 | High Positive | 0.111 |
| LOH: 1q42.12.1-1q42.13 | High Positive | 0.078 |

The next classification which is illustrated is the Orderings classification. This may be done by inferring the order of genetic alterations (step S224). The order of genetic alterations may be inferred by performing bulk cell sequencing and determining the proportion of cells comprising each genetic aberration. The aberrations present in a higher proportion of cells are determined to have occurred prior to the aberrations present in a lower proportion of cells. For instance, if we estimate that CHD1 LOH occurs in 90% of cancer cells and PTEN LOH occurs in 40% of cancer cells, then there must be cells that contain CHD1 LOH alone and CHD1 and PTEN LOH. Therefore, CHD1 LOH occurred first. The aberrations may be ranked in order of proportion.

The tumour may then be classified based on the determined order of the aberrations (step S226). As with FIGS. 15b to 15e, the SHapley Additive explanation (SHAP) value may be used to quantify the relative importance of the features when performing the classification using the gradient boosted decision tree method XGBoost. FIG. 15f illustrates the SHAP value for each feature when classifying a sample as belonging to Ordering I and FIG. 15g illustrates the SHAP value for each feature when classifying a sample as belonging to Ordering II. When generating these Figures, the ARBS score has been omitted as a feature for consistency with the other Figures.

For each of FIGS. 15f and 15g, each feature is ranked by its feature value and not unsurprisingly, there is a similar ranking in each Figure. Each feature with a high positive ranking for Ordering I has a high negative ranking for Ordering II and vice versa and the positive values show that the features are strongly suggestive of belonging to a particular Ordering:

### Features for Ordering I Classification

| Features | Ordering I | SHAP Value |
|---|---|---|
| Loss of heterozygosity: 21q22.2-21q22.3 | High Positive | 1.379 |
| Loss of heterozygosity: 16q12.1-16q24.3 | High Positive | 1.214 |
| Loss of heterozygosity: 17p | High Positive | 1.187 |
| Loss of heterozygosity: 8p | High Positive | 0.934 |
| Loss of heterozygosity: 10q23.1-10q25 | High Positive | 0.609 |
| ETS | High Positive | 0.457 |
| LOH: 12p13.32-12p12.3 | High Positive | 0.195 |
| Gain of whole chromosome 19 | High Positive | 0.165 |
| LOH: 19p13.3-19p13.2 | High Positive | 0.153 |

### Features for Ordering II Classification

| | | SHAP |
|---|---|---|
| Features | Ordering II | value |
| Loss of heterozygosity: 6q12-6q22.32 | High Positive | 2.555 |
| Loss of heterozygosity: 5q15-5q23 | High Positive | 1.896 |
| Percentage Genome Altered (subclonal component) | Med Positive | 1.230 |
| Loss of heterozygosity: 13q21.1-13q33.1 | High Positive | 1.228 |
| Loss of heterozygosity: 13q12.3-13q21.1 | High Positive | 0.759 |
| Gain: 8p23.3-8p22 | High Positive | 0.623 |
| Ratio of intra- to inter- chromosomal chained structural | Med Positive | 0.622 |
| variants | | |
| Gain: 9q12.9-9q21.11 | High Positive | 0.544 |
| Gain 8q | High Positive | 0.217 |
| LOH: 2q14.3-2q23.3 | High Positive | 0.210 |
| Gain: 3q21.2-3q29 | High Positive | 0.092 |
| LOH: 1q42.12.1-1q42.13 | High Positive | 0.083 |
| Chromothripsis | High Positive | 0.069 |

As suggested in the Figures and the table above, the genomic aberrations which may be indicative of the ordering classification include some or all of loss of heterozygosity in one or more of the regions 5q15-5q23.1 (spanning CHD1), 6q14.1-6q22.32 (MAP3K7, ZNF292), 8p (NKX3.1), 10q23.1-10q25.1 (PTEN), 13q12.3-13q21.1 (RB1, BRCA2) and 13q21.1-13q33.1 (EDNRB), 16q12.1-16q24.1 (CDH1) and 17p (TP53), SPOP mutations and ETS fusions. These are summarised in the table B below:

Table of features used for orderings classification

| | Indicative of | Indicative of |
|---|---|---|
| Feature (or chromosome region) | Ordering I | Ordering II |
| ETS gene fusion | Yes, when early | |
| SPOP mutation | | Yes, when early (but |
| | | less common) |
| 1q42.12-42.3 | Very early LOH | |
| 5q15-5q23.1 (spanning CHD1) | | LOH occurs early (but |
| | | less common) |
| LOH in 6q14.1-6q22.32 (MAP3K7, | | LOH occurs early |
| ZNF292) | | |
| 8p (NKX3.1) | LOH occurs early | LOH occurs early |
| LOH in 10q23.1-10q25.1 (PTEN) | LOH occurs | |
| 13q12.3-13q21.1 (RB1, BRCA2) | LOH occurs | LOH occurs early |
| 13q21.1-13q33.1 (EDNRB) | | LOH occurs early |
| 16q12.1-16q24.1 (CDH1) | LOH occurs | |
| 17p (TP53) | LOH occurs | |
| 19 | Late gain occurs | Late gain occurs (but |
| | | less common) |

The next step (step S230) may be to combine one or more of the clustering, ARBS and orderings classification to provide an overall classification for the tumour. Tumours which are classified as Alternative-evotype display poor prognosis. Each of a clustering classification as a metacluster MC-A, an ARBS classification of depleted and an orderings classification of Ordering-II are indicative of an overall classification as an Alternative-evotype. If all three intermediate classifications are used, the overall classification as an Alternative evotype is provided when the tumour has at least two intermediate classifications selected from classification as a metacluster MC-A, an ARBS classification of depleted and an orderings classification of Ordering-II. Similarly, each of a clustering classification as a metacluster MC-B1 or B2, an ARBS classification of enriched or indeterminate and an orderings classification of Ordering-I are indicative of an overall classification as a Canonical evotype. A tumour may be assigned to the Canonical evotype based on a similar majority-vote approach when at least two of the intermediate classifications are indicative of the Canonical evotype.

As an alternative to proceeding separately with each of the classifications, it is possible to classify the tumour directly as either a Canonical-evotype or an Alternative-evotype based on the presence of a combination of genomic aberrations. This can be used in combination with one or more of the classifications. Alternatively, as indicated by the dotted line, the method proceeds direct from receiving the data set at step S200 direct to the step of identifying genetic aberrations (step S232). As with the metacluster classification, a trained neural network can be used to process the raw inputs from new samples to generate the feature representation, and this can be used for assignment to one of the evotypes. Alternatively, we can use the raw inputs (or subset thereof) to develop a simple ML classifier that classifies by evotype directly as shown at step S234. For example, the SHapley Additive explanation (SHAP) value may be used to quantify the relative importance of the features when performing the classification using the gradient boosted decision tree method XGBoost. A value of zero indicates that the feature is not necessary to perform classification. FIG. 15h illustrates the SHAP value for classifying evotype directly. Comparing these features with those shown in FIG. 10e, there is considerable overlap with the highest ranked SHAP values. The overlapping features are listed in the table below according to the rank shown in FIG. 15h. Regarding the ARBS score, as shown in FIG. 15h, the SHAP value is significantly higher for this score and thus this is likely to be the most useful feature. As explained above, it can be used to indicate whether the tumour is a canonical or alternative evotype by considering the thresholds:

Table of features which may be used for direct classification
as Canonical or Alternative evotype:

| | Impact on | SHAP |
|---|---|---|
| Feature | model output | value |
| Normalised score of proximity from DNA breakpoints to nearest AR | High Positive | 4.487 |
| binding site (ARBS score) | | |
| Kataegis | High Negative | 1.798 |
| Ratio of intra- to inter- chromosomal chained structural variants | Med Positive | 1.044 |
| Percentage Genome Altered (clonal component) | Med Negative | 0.925 |
| Loss of heterozygosity of 5q11.1-5q14.1 | High Negative | 0.778 |
| Loss of heterozygosity of 6q12.6-6q22.32 | High Negative | 0.460 |
| Loss of heterozygosity of 5q15-5q23.1 | High Negative | 0.321 |
| Percentage Genome Altered (subclonal component) | High Negative | 0.300 |
| Gain of entire chromosome 7 | High Negative | 0.283 |
| Loss of heterozygosity of 16q12.1-16q24.1 | High Positive | 0.226 |
| Gain of 8p23.3-8p22 | High Negative | 0.223 |
| Gene fusion involving an ETS gene | High Positive | 0.180 |
| Loss of heterozygosity of 21q22.2-21q22.3 | High Positive | 0.160 |
| Loss of heterozygosity of 13q21.1-13q33.1 | High Negative | 0.156 |
| Loss of heterozygosity of 2q14.3-2q33.1 | High Negative | 0.153 |
| Chromothripsis | High Negative | 0.113 |
| Loss of heterozygosity of 17p | High Positive | 0.069 |
| Loss of heterozygosity of 18q | High Negative | 0.037 |
| Loss of heterozygosity: 12p13.32-12p12.3 | High Negative | 0.037 |
| Loss of heterozygosity of 8p | High Positive | 0.034 |
| LOH in 1p31.1-1p22.3 | High Negative | 0.031 |
| Gain in entire chromosome 19 | High Positive | 0.025 |
| SPOP | High Positive | 0.016 |
| Ploidy | High Positive | 0.012 |

The tumour can then be classified into a specific evotype using these features. It is likely to be to focus on a method and/or kit which targets a combination of the specific regions mentioned above. More general genome testing, e.g. to determine whether there is Chromothripsis or PGA, may be omitted from the kit and/or the method of classifying a subject to provide more rapid and simpler tests/methods. We can achieve 94.97% accuracy when classifying Canonical and Alternative-evotypes directly. The classification is then output at step S236, optionally with an associated probability that the assignment to the classification is accurate.

As will be appreciated, there is overlap between the features considered for each sub-classification (ARBS, clustering and orderings) and the direct classification. These are compared in the tables below and are ranked using the ranking in FIG. 15h. A Y is marked in the table if the aberration had a positive value in the corresponding Figure and its SHAP score was within 99% of the cumulative total.

TABLE 1
Genomic aberrations positively associated in SHAP value with Alternative
cancer evolutionary type (evotype) in sub-classifications

| Genomic | Type of | In meta cluster A | Indicative of | In ordering Il |
|---|---|---|---|---|
| aberration | aberration | classification? | ARBS depleted | classification? |
| Kataegis | Present | Y | Y | |
| PGA clonal | High | Y | | |
| 5q11.1-5q14.1 | Loss of | Y | Y | |
| (IL6ST, | heterozygosity | | | |
| PDE4D) | | | | |
| 6q12-6q22.32 | Loss of | Y | Y | Y |
| (MAP3K7, | heterozygosity | | | |
| ZNF292) | | | | |
| 5q15-5q23.1 | Loss of | Y | Y | Y |
| (CHD1) | heterozygosity | | | |
| Chromosome 7 | Gain | Y | Y | |
| 8p23.3-8p22 | Gain | | Y | Y |
| 13q21.1- | Loss of | | | Y |
| 13q33.1 | heterozygosity | | | |
| (EDNRB) | | | | |
| 2q14.3-2q23.3 | Loss of | Y | Y | Y |
| | heterozygosity | | | |
| Chromothripsis | Present | Y | Y | Y |
| 1q42.12- | Loss of | Y | Y | Y |
| 1q42.13 | heterozygosity | | | |
| 13q12.3- | Loss of | Y | | Y |
| 13q21.1 | heterozygosity | | | |
| (BRCA2, RB1) | | | | |
| SPOP | Mutation | Y | Y | |
| 3q21.2-3q29 | Gain | | Y | |
| 8q (MYC) | Gain | Y | Y | Y |

As shown in Table 1 above, the following genomic aberrations are present in all three sub-classifications: LOH in 6q12-6q22.32 (MAP3K7, ZNF292); LOH in 5q15-5q23.1 (CHD1), LOH in 2q14.3-2q23.3, Chromothripsis and LOH in 1q42.12-1q42.13. Thus, the presence of a combination of some or all of these features could be used to classify a subject in the first prognostic group, particularly a combination including at least the two highest ranked features which target specific regions within a genome, e.g. at least the top four: LOH in 6q12-6q22.32 (MAP3K7, ZNF292), LOH in 5q15-5q23.1 (CHD1), LOH in 2q14.3-2q23.3 and LOH in 1q42.12-1q42.13; more particularly at least the top two: LOH in 6q12-6q22.32 (MAP3K7, ZNF292); and LOH in 5q15-5q23.1 (CHD1). Similarly, the following genomic aberrations are present in at least two sub-classifications: Kataegis, LOH in 5q11.1-5q14.1 (IL6ST, PDE4D), Gain of whole chromosome 7, Gain in 8p23.3-8p22, LOH: 18q, LOH in 12p12.32-12p12.3, LOH in 13q12.3-13q21.1, SPOP, Gain in 8q (MYC). Thus, the presence of a combination of some or all of these features could be used to classify a subject in the first prognostic group, particularly a combination including at least the highest ranked features which target a specific region: LOH in 5q11.1-5q14.1 (IL6ST, PDE4D) and Gain of whole chromosome 7. The combinations for three and two subclassifications could be combined. For example, the presence of a combination including at least the highest ranked features targeting specific regions, e.g. LOH in 5q11.1-5q14.1 (IL6ST, PDE4D), LOH in 6q12-6q22.32 (MAP3K7, ZNF292) and LOH in 5q15-5q23.1 (CHD1) could be used to classify in the first prognostic group.

TABLE 2
Genomic aberrations positively associated in SHAP value
with Canonical cancer evotype in sub-classifications

| | | | Indicative of ARBS | |
|---|---|---|---|---|
| Genomic | Type of | In meta cluster | enriched or | In ordering I |
| aberration | aberration | B classification? | indeterminate | classification? |
| Inter/intra | High | Y | Y | |
| chromosomal | | | | |
| breakpoint ratio | | | | |
| ETS | Gene fusion | Y | Y | Y |
| 21q22.2- | Loss of | Y | Y | Y |
| 21q22.3 (ERG) | heterozygosity | | | |
| 17p (TP53) | Loss of | Y | Y | Y |
| | heterozygosity | | | |

As shown in Table 2 above, the following genomic aberrations are present in all three sub-classifications: ETS gene fusion and LOH in 17p. Thus, the presence of a combination of some or all of these features could be used to classify a subject in the second prognostic group, particularly a combination including at least the feature which targets a specific region, e.g. LOH in 17p. Similarly, the following genomic aberrations are present in at least sub-classifications: Inter/intra chromosomal breakpoint ratio and LOH in 21q22.2-21q22.3 (ERG). Thus, the presence of a combination of at least these features could be used to classify a subject in the second prognostic group. The combinations for three and two subclassifications could be combined. For example, the presence of a combination including at least the features which target specific regions, e.g. LOH in 17p and LOH in 21q22.2-21q22.3 (ERG) could be used to classify a tumour in the second prognostic group.

As an alternative to using the combinations described above, combinations based on the ranking of the proportion of tumours with the features shown in FIG. 10e could be used. For example, a combination including at least two of the features which target specific regions, e.g. LOH in 6q12-6q22.32, LOH in 13q21.1-13q33.1 and LOH in 13q12.3-13q21.1, could be used to classify a subject as belonging to the first prognostic group. For example, a combination including at least two of the features which target specific regions, e.g. LOH in 17p and LOH in 21q22.2-21q22.3 (ERG), could be used to classify a subject as belonging to the second prognostic group. It will be appreciated that these selections are merely included as examples and the top three, four, five or more features could be included.

In the various tables and description above, there are gene acronyms and these are listed below with the full gene name.

| Gene acronym | Full gene name |
|---|---|
| BPIFA4P | BPI fold containing family A member 4, pseudogene |
| BRCA2 | BRCA2 DNA repair associated |
| CDH1 | cadherin 1 |
| CHD1 | chromodomain helicase DNA binding protein 1 |
| CNKSR2 | connector enhancer of kinase suppressor of Ras 2 |
| COL2A1 | collagen type II alpha 1 chain |
| CRISP2 | cysteine rich secretory protein 2 |
| CXADRP2 | CXADR pseudogene 2 |
| DNAJC22 | DnaJ heat shock protein family (Hsp40) member C22 |
| EDNRB | endothelin receptor type B |
| EGFR | epidermal growth factor receptor |
| ELK4 | ETS transcription factor ELK4 |
| ERG | ETS transcription factor ERG |
| ETV1 | ETS variant transcription factor 1 |
| ETV3 | ETS variant transcription factor 3 |
| ETV4 | ETS variant transcription factor 4 |
| ETV5 | ETS variant transcription factor 5 |
| ETV6 | ETS variant transcription factor 6 |
| FLI1 | Fli-1 proto-oncogene, ETS transcription factor |
| GM-CSF | colony-stimulating factor 2 |
| HAUS1P2 | HAUS augmin like complex subunit 1 pseudogene 2 |
| HSD17B11 | hydroxysteroid 17-beta dehydrogenase 11 |
| IFNA2 | interferon alpha 2 |
| IGHA2 | immunoglobulin heavy constant alpha 2 (A2m marker) |
| IL-2 | interleukin 2 |
| IL6ST | interleukin 6 cytokine family signal transducer |
| MAP3K7 | mitogen-activated protein kinase kinase kinase 7 |
| MYC | MYC proto-oncogene, bHLH transcription factor |
| NCALD | neurocalcin delta |
| NKX3.1 | NK3 homeobox 1 |
| NLRP9 | NLR family pyrin domain containing 9 |
| OGDHL | oxoglutarate dehydrogenase L |
| PDE4D | phosphodiesterase 4D |
| PTEN | phosphatase and tensin homolog |
| RB1 | RB transcriptional corepressor 1 |
| RIMBP2 | RIMS binding protein 2 |
| SPOP | speckle type BTB/POZ protein |
| TDRD1 | tudor domain containing 1 |
| TMPRSS2 | transmembrane serine protease 2 |
| TP53 | tumor protein p53 |
| ZNF292 | zinc finger protein 292 |

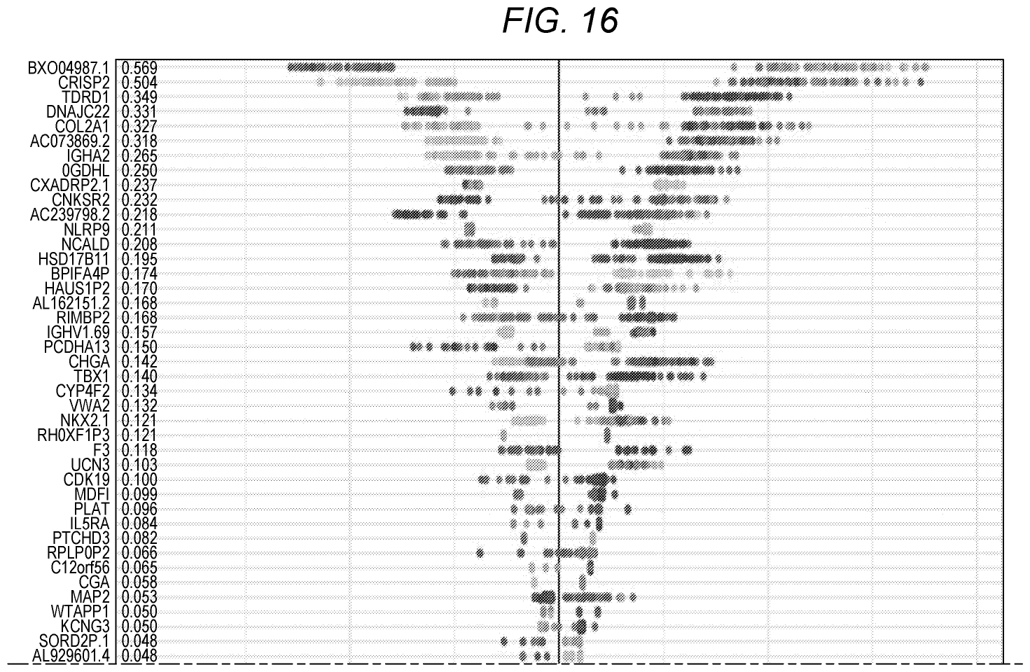

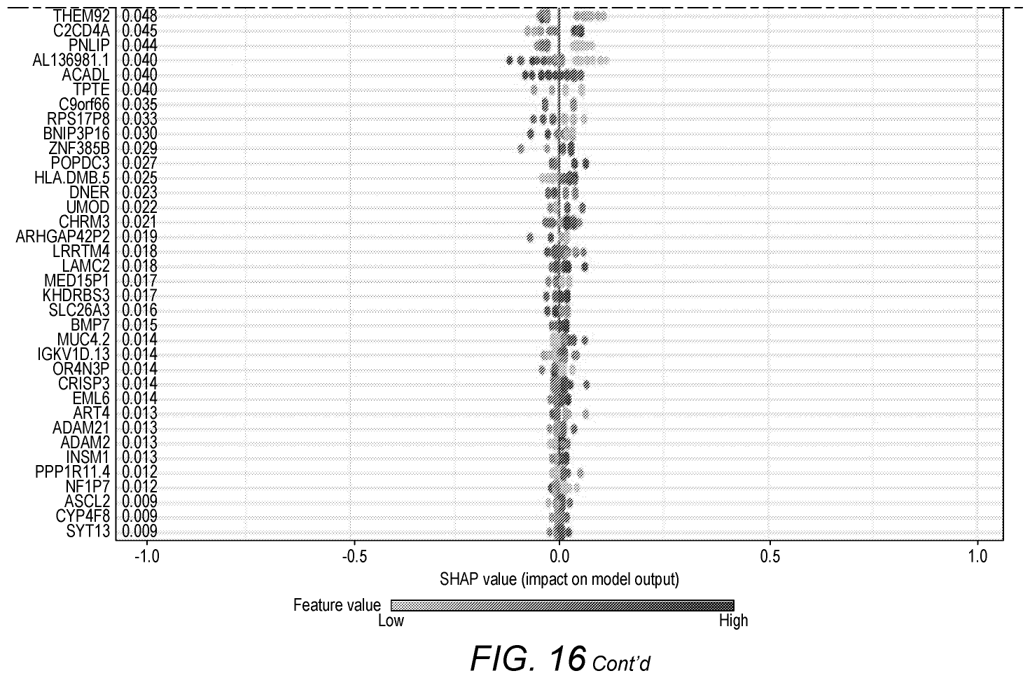

FIG. 16 illustrates that evotypes could also be classified using other technologies. For example, we have RNA-seq from tumour and adjacent normal tissue for 136 of the 159 samples used to derive the Evotypes. Performing differential gene expression analysis using the EdgeR package between reveals that there are 588 genes that are significantly differentially expressed (adjusted P-value<0.05) between the Canonical and Alternative Evotypes. This set can potentially be used as a basis for classifying by Evotype. Performing the classification with XGBoost, we find we get an 84.56% classification accuracy. Calculating the SHAP values for this classifier we find 77 variables with a non-zero SHAP value as shown in FIG. 16.

This is still quite a large number of parameters to search in the XGBoost algorithm, and so we attempt to optimise the classification by reducing the number of inputs further by finding the set of transcripts with the highest SHAP values that maximises the classification accuracy. Through this method we find that we can obtain a maximal classification accuracy of 91.91% when classifying using the top 18 transcripts. These are listed in the table below:

Table of features from RNA expression
which can be used for classification:
Feature

BX004987.1
CRISP2
TDRD1
DNAJC22
COL2A1
AC073869.2
IGHA2
OGDHL
CXADRP2.1
CNKSR2
AC239798.2
NLRP9
NCALD
HSD17B11
BPIFA4P
HAUS1P2
AL162151.2
RIMBP2

Therefore, we conclude that Evotypes can be directly classified with information from RNA expression. Furthermore, using the full set of 77 transcripts we can also obtain a 94.12% accuracy in the classification of tumour and benign samples using XGBoost.

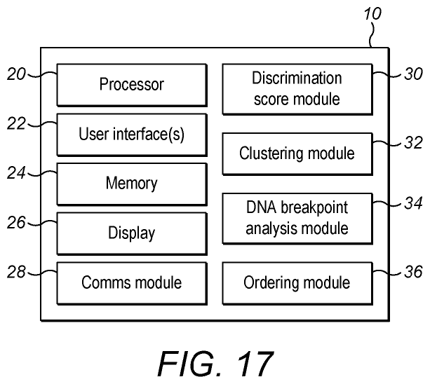

FIG. 17 is a schematic of an associated system for performing the computer-implemented aspects of the methods described above (both the discovery and the classification). The system comprises a computing device 10 which could be a handheld device which is portable for a clinician to transport from patient to patient and an app could be loaded onto the device for performing the predictions. The computing device 10 comprises the standard components such as a processing unit or processor 20, a user interface unit 22 for allowing a user to input information and a memory 24. The user interface may display information or alternatively, there may be a display 24 for displaying information to a user, e.g. a suggestion for treatment as described above. There may also be a communications module 28 for communicating with other devices and/or accessing the cloud, e.g. to process the data as described below.

The computing device 10 also has a discrimination score module 30 for calculating a discrimination score, a clustering module 32 for determining a clustering classification, a DNA breakpoint analysis module 34 for analyzing the location of breakpoints within the sequence whereby the ARBS classification can be determined and an ordering module 36 for determining an ordering classification as described above. Each of the modules may be stored in the memory 24 or in separate storage on the device (not shown). The modules may also be stored remotely from the computing device 10 for example in the cloud.

This schematic system may be constructed, partially or wholly, using dedicated special-purpose hardware. Terms such as ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components discussed herein, such functional elements may be combined into fewer elements or separated into additional elements.

### SUMMARY

As described above a comprehensive analysis of genomic measurements from 159 prostate cancer patients using three statistical and machine-learning methods has been performed. This analysis identified two distinct forms of prostate cancer evolutionary types, referred to herein as “evotypes”, which can be characterised by various characteristics. Firstly, the evotypes can be characterised by location of double stranded DNA breaks relative to androgen receptor binding sites (an ARBS classification as described above). Secondly, the evotypes can be characterised by certain genetic aberrations and combinations of certain genetic aberrations (e.g. using the clustering classification or orderings classification as described above). The evotypes may be characterised by the combination of the location of DNA double stranded breaks and the genetic aberrations.

Stratification by evotype could have epidemiological implications. For instance, non-Caucasian racial groups display an increased incidence of many Alternative-evotype aberrations (27-29) and may therefore have a higher predisposition for this disease type. Conversely, cancers arising in younger patients have enrichment for ARBS-proximal breakpoints (17), and are reported to develop via a similar evolutionary progression to the Canonical-evotype (14, 17). It may also be possible to tailor treatment strategies to each evotype. In particular, cancers with Alternative-evotype aberrations have been shown to be susceptible to ionising radiation (22) and have a better response to treatment with PARP inhibitors (30) and androgen ablation (23). Our model for prostate cancer evolutionary disease types therefore provides a conceptual framework that unifies the results of many previous studies and has far-reaching implications for our understanding of disease progression, prognosis and treatment.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

All documents and references to Gene/protein accession numbers mentioned in this specification are incorporated herein by reference in their entirety. “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

1. A method for treating prostate cancer in a subject in need thereof, the method comprising:
(a) stratifying a subject having cancer or metastatic disease into one of two prognostic groups, wherein said stratifying comprises:
analysing, using DNA and/or RNA sequencing, a biological sample obtained from the subject with cancer or metastatic disease;
determining, in the biological sample, locations of double stranded DNA breakpoints relative to androgen receptor binding sites (ARBS);
calculating an ARBS score for the biological sample by comparing proximity of the determined locations to ARBS for the biological sample to proximity of a baseline distribution of double stranded DNA breakpoint locations to ARBS; and
classifying, using the ARBS score, the subject:
in a first prognostic group when the ARBS score indicates that the determined locations are less frequently proximal to androgen receptor binding sites than the baseline distribution, or
in a second prognostic group when the ARBS score indicates that the determined locations are equally or more frequently proximal to androgen receptor binding sites than the baseline distribution; and
(b) treating the subject based on the prognostic group in which the subject is classified.

2. The method of claim 1, further comprising classifying the subject in the second prognostic group when ARBS score indicates there is no statistically significant difference between proximity of the determined locations to the androgen receptor binding sites and proximity of expected locations of breakpoints to the androgen receptor binding sites.

3. The method of claim 1, further comprising defining the baseline distribution of breakpoint locations by randomly shuffling observed breakpoints in sample data.

4. The method of claim 1, further comprising calculating the ARBS score by:
determining the proportion of the determined locations which are less than a threshold number of base pairs from an androgen receptor binding site;
obtaining the proportion of the breakpoint locations in the baseline distribution which are less than a threshold number of base pairs from an androgen receptor binding site, and
normalising the determined proportion by the obtained proportion to obtain the ARBS score to determine whether the determined locations are more frequently proximal or less frequently proximal to androgen receptor binding sites than the baseline distribution.

5. The method of claim 4, comprising classifying the subject in the second prognostic group when the ARBS score is greater than an upper threshold.

6. The method of claim 4, comprising classifying the subject in the first prognostic group when the ARBS score is less than a lower threshold.

7. The method of claim 1, further comprising:
identifying further genomic aberrations present in the sample; and
further classifying the subject:
in the first prognostic group based on the presence of one or more genomic aberrations selected from the group consisting of loss of heterozygosity in one or more of the following regions: 1q42.12-1q42.13, 2q14.3-2q23.3, 5q11.1-5q23.1 (IL6ST, PDE4D), 5q15-5q23.1 (CHD1), 6q12-6q22.32 (MAP3K7, ZNF292), 13q12.3-13q21.1 (BRCA2, RB1), and 13q13.3-13q33.1 (EDNRB); gain of heterozygosity in one or more of the following regions: 3q21.2-3929, Chromosome 7, 8p23.3-8p22, and 8q (MYC); SPOP mutations; kataegis; chromothripsis; and Percentage Genome Altered (clonal component); and
in the second prognostic group based on the presence of one or more genomic aberrations selected from the group consisting of loss of heterozygosity in one or more of the following regions: 17p (TP53), 19p13.3-19p13.2, and 21922.2-21922.3 (ERG); ETS gene fusions; and the ratio of inter to intra chromosomal breakpoints.

8. The method of claim 7, comprising
classifying the subject in the first prognostic group based on the presence of the combination of genomic aberrations including ARBS score, and loss of heterozygosity in at least the regions: 6q12-6q22.32 (MAP3K7, ZNF292) and 5q15-5q23.1 (CHD1) and
classifying the subject in the second prognostic group based on the presence of the combination of genomic aberrations including ARBS score and loss of heterozygosity in the regions: 16q12.1-16q24.3 and 17p.

9. The method of claim 1, further comprising using a clustering classification to classify the subject in the first prognostic group based on the presence of one or more genomic aberrations selected from a set of genomic aberrations including loss of heterozygosity in regions 1q42.12-1q42.13, 2q14.3-2q23.3, 5q11.1-5q14.1 (IL6ST, PDE4D), 5q15-5q23.1 (spanning CHD1), 6q14.1-6q22.32 (MAP3K7, ZNF292), 12p12.32-12p12.3 and 18q, gain in the whole chromosome 7 and in region 8q, kataegis, SPOP mutations, and Percentage Genome Altered (clonal component).

10. The method of claim 1, further comprising using a clustering classification to classify the subject in the second prognostic group based on the presence of one or more genomic aberrations selected from a set of genomic aberrations including ETS gene fusions, the ratio of inter to intra chromosomal breakpoints, Percentage Genome Altered (subclonal component), loss of heterozygosity in regions 17p (TP53), 16q12.1-16q24.3 and 22q11.21-22q11.22 and gain in regions 9q12.9-9q21.11 and whole chromosome 19.

11. (canceled)

12. The method of claim 1, further comprising
analysing the biological sample using bulk cell sequencing:
determining the proportion of cells in the biological sample having one or more genomic aberrations;
identifying an order in which the genomic aberrations occurred by determining that the genomic aberrations which are present in a larger proportion of cells occurred before the genomic aberrations which are present in a smaller proportion of cells; and
classifying the subject in one of the first and second prognostic groups using an orderings classification based on the identified order;
wherein the genomic aberrations include at least one or more of loss of heterozygosity in one or more of the regions: 5q15-5q23.1 (spanning CHD1), 6q14.1-6q22.32 (MAP3K7, ZNF292), 8p (NKX3.1), 10q23.1-10q25.1 (PTEN), 13q12.3-13q21.1 (RB1, BRCA2), 13q21.1-13q33.1 (EDNRB), 16q12.1-16q24.1 (CDH1), 17p (TP53) and 21q22.2-21q22.3, gain in one or more of the regions: 8p23.3-8p22 and 9q12.9-9q21.11, Percentage Genome Altered (subclonal component), Ratio of intra- to inter-chromosomal chained structural variants and ETS fusions.

13. (canceled)

14. A method according to claim 12, comprising classifying the subject in the first prognostic group when the genomic aberrations include at least one or more of loss of heterozygosity in one or more of the regions: 5q15-5q23.1 (spanning CHD1), 6q14.1-6q22.32 (MAP3K7, ZNF292), 13q12.3-13q21.1 (RB1, BRCA2), 13q21.1-13q33.1 (EDNRB), gain in one or more of the regions: 8p23.3-8p22 and 9q12.9-9q21.11, Percentage Genome Altered (subclonal component) and ratio of intra- to inter-chromosomal chained structural variants.

15. A method according to claim 14, comprising classifying the subject in the first prognostic group based on the presence of the combination of genomic aberrations including loss of heterozygosity in the regions: 5q15-5q23.1 (spanning CHD1), 6q14.1-6q22.32 (MAP3K7, ZNF292), 13q12.3-13q21.1 (RB1, BRCA2), 13q21.1-13q33.1 (EDNRB) and Percentage Genome Altered (subclonal component).

16. A method according to claim 12, comprising classifying the subject in the second prognostic group when the genomic aberrations include at least one or more of loss of heterozygosity in one or more of the regions: 8p (NKX3.1), 10q23.1-10q25.1 (PTEN), 16q12.1-16q24.1 (CDH1), 17p (TP53) and 21q22.2-21q22.3, and ETS fusions.

17. A method according to claim 16, comprising classifying the subject in the second prognostic group based on the presence of the combination of genomic aberrations including loss of heterozygosity in the regions: 8p (NKX3.1), 10q23.1-10q25.1 (PTEN), 16q12.1-16q24.1 (CDH1), 17p (TP53) and 21q22.2-21q22.3.

18. A method according to claim 12, further comprising:
identifying further genomic aberrations present in the sample;
using a clustering classification to classify the subject in the first prognostic group based on the presence of one or more genomic aberrations selected from a set of genomic aberrations including loss of heterozygosity in regions 1q42.12-1q42.13, 2q14.3-2q23.3, 5q11.1-5q14.1 (IL6ST, PDE4D), 5q15-5q23.1 (spanning CHD1), 6q14.1-6q22.32 (MAP3K7, ZNF292), 12p12.32-12p12.3 and 18q, gain in the whole chromosome 7 and in region 8q, kataegis, SPOP mutations, and Percentage Genome Altered (clonal component);
using a clustering classification to classify the subject in the second prognostic group based on the presence of one or more genomic aberrations selected from a set of genomic aberrations including ETS gene fusions, the ratio of inter to intra chromosomal breakpoints, Percentage Genome Altered (subclonal component), loss of heterozygosity in regions 17p (TP53), 16q12.1-16q24.3 and 22q11.21-22q11.22 and gain in regions 9q12.9-9q21.11 and whole chromosome 19; and
determining an overall classification as the first prognostic group when at least two of the ARBS, clustering and ordering classifications classify the subject in the first prognostic group and as the second prognostic group when at least two of the ARBS, clustering and ordering classifications classify the subject in the second prognostic group.

19. The method of claim 1, further comprising:
identifying further genomic aberrations present in the sample; and
classifying the subject in the first prognostic group based on the presence of one or more genomic aberrations selected from loss of heterozygosity in one or more of the following regions: 2q14.3-2q23.3, 5q11.1-5q14.1 (IL6ST, PDE4D), 5q15-5q23, 6q12-6q22.32 (MAP3K7, ZNF292), 18q, gain of heterozygosity in one or more of the following regions: 3q21.2-3q29, whole chromosome 7, 8p23.3-8p22, 8q, 9q12.9-9q21.11, kataegis.

20. The method of claim 19, comprising confirming the classification of the subject in the second prognostic group based on the presence of one or more genomic aberrations selected from the group comprising loss of heterozygosity in one or more of the following regions: 10q23.1-10q25, 16q12.1-16q24.3, 17p, gain of heterozygosity in one or more of the following regions: whole chromosome 19, ratio of intra- to inter-chromosomal chained structural variants, ETS, Percentage Genome Altered (subclonal component) and Percentage Genome Altered (clonal component).

21. A method for treating prostate cancer in a subject in need thereof, the method comprising:
(a) stratifying a subject having cancer or metastatic disease into one of two prognostic groups, wherein said stratifying comprises:
analysing, using DNA and/or RNA sequencing, a biological sample obtained from the subject with cancer or metastatic disease,
identifying genomic aberrations in the biological sample, and
classifying the subject:
in a first prognostic group based on the presence of a combination of genomic aberrations including ARBS score and loss of heterozygosity in at least the regions: 6q12-6q22.32 (MAP3K7, ZNF292) and 5q15-5q23.1 (CHD1); or
in a second prognostic group based on the presence of a combination of genomic aberrations including ARBS score and loss of heterozygosity in the regions: 16q12.1-16q24.3 and 17; and
(b) treating the subject based on the prognostic group in which the subject is classified.

22-27. (canceled)

28. The method of claim 1, wherein the subject is identified for treatment selected from one or more of external beam radiation, brachytherapy, radical prostatectomy, hormone therapy, and/or chemotherapy when stratified in the first prognostic group.

29. The method of claim 1, wherein the subject is selected for patient surveillance when stratified as belonging to the second prognostic group.

30-34. (canceled)
